(19) United States
(12) Patent Application Publication
(10) Pub. No.: US 2013/0209522 A1
(43) Pub. Date: Aug. 15, 2013

(54) Drug Release from a Polymer-Controlled Local Antibiotic Delivery System Using a Degradable Bone Graft

(71) Applicant: UNIVERSITY OF UTAH RESEARCH FOUNDATION, (US)

(72) Inventors: Amanda Elaine Brooks, South Jordan, UT (US); Benjamin Delbert Brooks, South Jordan, UT (US); David W. Grainger, Salt Lake City, UT (US)

(73) Assignee: UNIVERSITY OF UTAH RESEARCH FOUNDATION, Salt Lake City, UT (US)

(21) Appl. No.: 13/759,904

(22) Filed: Feb. 5, 2013

Related U.S. Application Data
(60) Provisional application No. 61/595,544, filed on Feb. 6, 2012.
(60) Provisional application No. 61/616,937, filed on Mar. 28, 2012.

(51) Int. Cl. A61K 47/46; A61K 31/431; A61K 38/14; A61K 31/496; A61K 31/7036; A61K 47/34

(52) U.S. Cl. CPC A61K 47/46; A61K 31/7036; A61K 47/34; A61K 38/14; A61K 31/496; A61K 31/431
USPC 424/400; 514/40; 514/2.3; 514/253.08; 514/254.11; 514/196; 264/330

(57) ABSTRACT

In some embodiments, the invention provides an implant comprising a uniform mixture of degradable polymer, bone, and a drug. In some embodiments, the drug comprises an antibiotic. In some embodiments, diffusion of the drug from the implant at a therapeutic level is maintained for an amount of time longer than an amount of time that a pathogen is senescent. In some embodiments, diffusion of the drug from the implant at a therapeutic level is maintained for at least eight weeks, or at least ten weeks, or at least twelve weeks post-implantation. In some embodiments, the therapeutic level is maintained at an implantation site of the implant.

In some embodiments, the implant is a solid, a paste, or a liquid. In some embodiments, the solid implant is carved or molded for insertion into a site of implantation in a vertebrate host prior to implantation. In some embodiments, the paste implant hardens following implantation. In some embodiments, the liquid implant is used to coat a prosthesis (e.g., a prosthesis made of a metal, a ceramic, a porcelain, or a combination of two or more of the foregoing).

### REFERENCE TO RELATED APPLICATION

This patent application claims benefit of U.S. provisional application Ser. No. 61/616,937, filed Mar. 28, 2012 and U.S. provisional application Ser. No. 61/595,544, filed Feb. 6, 2012, the entireties of both applications are hereby incorporated by reference.

### TECHNICAL FIELD

The present invention relates to the fields of medical devices, biology and medicine, and more particularly to the field of implantable biomaterials and combination medical devices.

### BACKGROUND

Each year in the United States alone, over 500,000 orthopedic surgeries are performed (Bostrom and Seigerman, Hss J, vol. 1, pp. 9-18, 2005; Giannoudis et al., Injury 36, suppl. 3, ppS20-7, 2005), many of which require the use of a natural or engineered bone graft to fill a traumatic or surgically-induced wound or defect (Early Radiological Diagnosis and Differential Diagnosis of Infection in Orthopaedic Surgery,” in Infection and Local Treatment in Orthopedic Surgery, E. Meani, C. Romanò, L. Crosby, G. Hofmann, and G. Calonego, Eds.: Springer, 2007). Cadaveric-sourced allograft bone (e.g., cancellous allograft fragment and morsellized, micron-sized particulate matter) is often used due to not only its high surface area, that provides an appropriate cellular environment to enhance tissue integration and bone remodeling (Nandi et al., Indian J. Med. Res. 132: 15-30, 2010; Kundu et al., J. Mater. Sci. Mater. Med 21: 2955-2969, 2010) but also its wound packing efficiency that minimizes the occurrence of avascular spaces susceptible to opportunistic bacterial colonization (Nandi et al., supra; Aronin et al., Biomaterials 31: 6417-6424; Kanellakopoulou and Giamarellos-Bourboulis, Drugs, vol. 59, pp. 1223-32, 2000; and Winkler et al., J Bone Joint Surg Br, vol. 90, pp. 1580-4, 2008).

Synthetic bone fillers (e.g., calcium phosphate granules, calcium sulfate-based granules, wafers, pastes, and polymers), or naturally derived bone replacement materials such as ProOsteon 500R (BioMet), a porous hybrid calcium carbonate/calcium phosphate coralline ceramic bone graft (Parikh, S. N., J Postgrad Med, 48 (2002) 142-148), and bioactive bone-based technologies (e.g., osteo-inductive growth factors, drug carriers) provide new surgical options with novel, ‘ala carte’ orthopedic solutions for trauma, revision surgeries and major repairs (Giannoudis et al., supra; McLaren, A. C., Clin Orthop Relat Res, (2004) 101-106).

Regardless of the type of implant, clinical success of bone graft void fillers relies on their ability to properly pack the orthopedic defect and allow adequate vascularization for graft integration via tissue, primarily bone, regeneration. Importantly, the intrinsic low vascularity of bone and persisting presence of susceptible avascular spaces provides a favorable niche for acute and chronic bacterial infection. Over 40,000 infected orthopedic surgeries occur per year, many of which are related to bacterial biofilm (see FIG. 1A). The surgical sites that are prone to infection occur during replacement of, for example, diseased bone (FIG. 1B) or infected artificial joints (FIG. 1C). Most alarmingly, the recurrence of infection rate is 20-30%. Overall, over 1000 patient deaths occur annually.

The high revision infection rates are particularly troublesome. For hip and knee replacement infections, 1-3% of primary joint replacements become infected, but 8-15% of revision arthroplasty surgeries become infected. For knee revisions, over 54,000 primary surgeries occur per year, with a 9% revision rate (compare “TKA” lines of FIG. 2A with FIG. 2B), with 15% becoming infected and the infection recurrence rate is 20-30%. For open tibia fraction revisions, there is a 35% reoperation rate (compare “THA” lines of FIG. 2A with FIG. 2B), with a 10-50% type III fracture infection rate and 11% bone grafts. Similarly, in osteomyelitis, bacterial contamination can occur for example, in large bone defects, deep surgical site infections, and/or situations where infections and poor blood supply compromise healing (see FIG. 3).

These infectious events, particularly those that lead to biofilm formation, can further inhibit graft revascularization and proper cortical blood supply, leading not only to tissue necrosis (sequestra) but also to additional avascular spaces (Costerton, J. W., Rev Infect Dis, 6 (1984) 608-616). The porosity and resulting high surface area enables cancellous allograft bone fragments or morselized allograft bone as well as their synthetic surrogates (McKee et al., J. Orthop Trauma, 16 (2002) 622-627; Koort et al., Acta Orthop, 79 (2008) 295-301; and Koort et al., J. Biomed Mater Res A, 78 (2006) 532-540) to be exploited clinically both as a suitable bone substitute and filler and importantly as a local drug delivery vehicle to prevent or treat osteomyelitis, with the degree of porosity directly correlating to antibiotic loading efficiency (Nandi et al., supra; Aronin et al., Biomaterials 31: 6417-6424; Kanellakopoulou and Giamarellos-Bourboulis, Drugs, vol. 59, pp. 1223-32, 2000; and Winkler et al., J Bone Joint Surg Br, vol. 90, pp. 1580-4, 2008). Cancellous autogenic bone grafts and synthetic bone grafts impregrated with antibiotic prior to implantation have been reported to show a reduction in infection with no clinical contraindications, further indication that endowing clinically familiar cancellous allograft bone tissue with application-tailored, polymer-controlled antibiotic release may provide a delivery vehicle to effectively treat osteomyelitis (see, e.g., Borkhuu et al., Spine (Phila Pa 1976), 33 (2008) 2300-2304; Buttaro et al., Acta Orthop Scand, 74 (2003) 505-513; Finley, J. M., J West Soc Periodontol Periodontal Abstr, 49 (2001) 5-9; Winkler et al., Cell Tissue Bank, 7 (2006) 319-323; Witso et al., Acta Orthop Scand, 70 (1999) 298-304; Ketonis et al., Clin Orthop Relat Res, 468 (2010) 2113-2121).

However, simple antibiotic adsorption often used with bone graft materials produces rapid bolus release and limited therapeutic duration of up to a few days maximum (Jiranek et al., J Bone Joint Surg Am, 88 (2006) 2487-2500; Ketonis, et al., Tissue Eng Part A, 16 (2010) 2041-2049; M. Diefenbeck et al., Injury, 37 Suppl 2 (2006) S95-104; Ciampolini and Harding, Postgrad Med J, 76 (2000) 479-483; Levin, P. D., J Bone Joint Surg Br, 57 (1975) 234-237; and Miclau et al., J Orthop Res, 11 (1993) 627-632). This rapid bolus drug release often cannot stem long-term infections where opportunistic pathogens reside in the wound site several weeks to months.

As shown in FIGS. 4A-4C, formation of a bacteria biofilm blocks systemic antibiotic delivery. Biofilm-resident pathogens are refractory to antibiotic treatments because they maintain mixed microbial populations within this matrix composed of low metabolism and senescent organisms intrinsically unaffected by antimicrobial treatment since they are not metabolically active. Antibiotic treatments of short durations are effective against active metabolic pathogens. However, when short-lived antibiotic dosing currently applied dissipates, senescent pathogens can “awaken” within the biofilm, producing a second tier of persistor cells capable of producing infection beyond the residence time of most locally administered antimicrobial agents. Acute therapy cannot effectively treat biofilm resident organisms, demanding a time-release, extended antibiotic regime to address initially senescent persistor organisms. Thus, biofilm bacteria are 10-1000 times less susceptible to antibiotics than non-biofilm bacteria.

Currently used treatments fill bone defects with antibiotic-containing cement. However, this short release duration of the antibiotic is inadequate to resolve infection. Moreover, the current rapid bolus release approaches have resulted in acute local tissue toxicity and development of drug-resistant and multi-drug resistant organisms (FIGS. 6A and O.o t.A.D.f. Communication, “CDC Urges Hospitals and Healthcare Facilities to Increase Efforts to Reduce Drug-Resistant Infections,” 2006). Additionally, once the bolus release has been exhausted, the now biologically silent cements 1) continue to leach antibiotic at a level that promotes antibiotic-resistant bacteria and (2) acts as a local foreign body and nidus of infection.

Thus, there is a need to find improved compositions and methods for treating orthopedic injuries (e.g., bone and joint injuries, and implant wounds) to produce better healing of tissues and bone implant sites while reducing the risks of long-term infection.

### SUMMARY OF THE EMBODIMENTS

The invention provides compositions and methods for treating bone and joint injuries while reducing the risks of long-term infection.

Accordingly, in a first aspect, the invention provides an implant comprising, consisting, or consisting essentially of a uniform mixture of degradable polymer component, a bone component, and a drug component. In some embodiments, the drug component comprises, consists, or consists essentially of an antibiotic.

In some embodiments, the implant is configured so that upon implantation of the implant into a host at an implantation site, the drug diffuses from the implant at a therapeutic level (e.g., a level that will inhibit or prevent infection at the implantation site). In some embodiments, the host is a vertebrate animal. In some embodiments, diffusion of the drug from the implant at a therapeutic level is maintained for at least eight weeks post-implantation. In some embodiments, diffusion of the drug from the implant at a therapeutic level is maintained for at least ten weeks post-implantation. In some embodiments, diffusion of the drug from the implant at a therapeutic level is maintained for at least twelve weeks post-implantation. In some embodiments, the therapeutic level is maintained at an implantation site of the implant.

In some embodiments, the bone component is natural bone. In some embodiments, the bone component is synthetic bone. In some embodiments, the bone component comprises, consists, or consists essentially of bone fragments (e.g., fragments of synthetic bone or fragments of natural bone). In some embodiments, the bone component comprises ground or morselized bone (e.g., morselized synthetic bone or morselized natural bone).

In some embodiments, the implant is a solid. In some embodiments, the implant is molded (e.g., is a putty that can be molded). In some embodiments, the implant is injected as a paste. In some embodiments, the liquid or paste implant hardens upon implantation. In some embodiments, the implant is carveable, so that it may be shaped prior to implantation. In some embodiments, the implant is shaped for use with an implantable prosthesis. In some embodiments, the prosthesis is a fixation tooling, a plate, a screw, a rod, a pin, a nail, or a total arthroplasty of various forms used clinically in orthopedic surgery.

In some embodiments, the implant is a liquid. In some embodiments, the liquid implant is a coating on an implantable prosthesis. In some embodiments, the prosthesis is of a material selected from the group consisting of a metal (including, for example, a metal oxide), a ceramic, a porcelain, an alloy, and a combination of two or more of the foregoing.

In some embodiments, the implant is configured so that upon implantation of the implant, the drug diffuses from the implant in a manner to provide a first bolus after a first period of time following implantation and a second bolus after a second period of time following implantation. In some embodiments, the first period is about one week and the second period is about five weeks. In some embodiments, the first period is about one day and the second period is between about three weeks and about six weeks.

In some embodiments, the degradable polymer comprises, consists, or consists essentially of a polycaprolactone (PCL) polymer (e.g., PCL of various different molecular weights). In some embodiments, the degradable polymer comprises, consists, or consists essentially of a polyethylene glycol (PEG) polymer. In some embodiments, the degradable polymer comprises, consists, or consists essentially of a poly(lactide-co-glycolide) polymer. In some embodiments, the implant further comprises a poragen such as calcium chloride. In some embodiments, the implant is contiguously porous.

In some embodiments of the implant, the bone is present in the uniform mixture in a first quantity by weight and the degradable polymer is present in the uniform mixture in a second quantity by weight, wherein the first quantity is greater than the second quantity. In some embodiments, the first quantity is at least 1.125 times larger than the second quantity, or is at least 1.25 times larger than the second quantity, or is at least 1.5 times larger than the second quantity, or is at least two times larger than the second quantity, or is at least 2.25 times larger than the second quantity, or is at least 2.5 times larger than the second quantity, or is at least 4 times larger than the second quantity, or is at least 5 times larger than the second quantity.

In another aspect, the invention provides a method of making a solid implant, the method comprising: making a uniform mixture including degradable polymer, bone, and a drug; forming the mixture into a desired shape; and curing the shaped mixture to form a solid implant. In some embodiments, the curing step includes subjecting the shaped mixture to heat. In some embodiments, the curing step includes subjecting the shaped mixture to sterilization. In some embodiments, the implant is contiguously porous.

In various embodiments, the bone is present in the uniform mixture in a first quantity by weight and the degradable polymer is present in the uniform mixture in a second quantity by weight, wherein the first quantity is greater than the second quantity. In some embodiments, the first quantity is at least 1.125 times larger than the second quantity, or is at least 1.25 times larger than the second quantity, or is at least 1.5 times larger than the second quantity, or is at least two times larger than the second quantity, or is at least 2.25 times larger than the second quantity, or is at least 2.5 times larger than the second quantity, or is at least 4 times larger than the second quantity, or is at least 5 times larger than the second quantity.

In another aspect, the invention provides an implantable bone void filler comprising, consisting, or consisting essentially of a polymer component, an antibiotic, and a bone fragment (e.g., a bone fragment from a natural cadaver bone source or a synthetic bone fragment). In some embodiments, the filler further comprises a poragen such as calcium chloride. In some embodiments, the filler is contiguously porous. In some embodiments, the polymer component comprises or consists ofpolycaprolactone (PCL) (e.g., PCL of various different molecular weights). In some embodiments, the polymer component comprises, consists, or consists essentially of polyethylene glycol. In some embodiments, the polymer component comprises, consists, or consists essentially of poly(lactide-co-glycolide) polymer. In some embodiments, the polymer component comprises, consists, or consists essentially of aa combination of PEG and PCL. In some embodiments, the polymer component comprises, consists, or consists essentially of a combination of PEG, PCL, and poly(lactide-co-glycolide) In some embodiments, the antibiotic is selected from the group consisting of tobramycin, ciprofloxacin, and vancomycin.

### BRIEF DESCRIPTION OF THE DRAWINGS

The patent or application file contains at least one drawing executed in color. Copies of this patent with color drawing(s) will be provided by the Patent and Trademark Office upon request and payment of the necessary fee.

The patent or application file contains at least one drawing executed in color. Copies of this patent or patent application publication with color drawing(s) will be provided by the Office upon request and payment of the necessary fee.

The foregoing features of embodiments will be more readily understood by reference to the following detailed description, taken with reference to the accompanying drawings, in which:

FIGS. 1A-1C are images showing infections of bone. FIG. 1A is an animated representation showing a biofilm related infection of a joint. FIG. 1B are X-ray images of a septic non-union infection (left image) and an aseptic non-union infection. FIG. 1C is a schematic overlying an X-ray of a hip replacement showing the hot-spot for infection.

FIG. 2A is a line graph showing the annual number of procedures of primary hip replacement surgeries (“THA” line, open circle) and primary knee replacement surgeries (“TKA” line, no circles). This figure originally appeared in Kutz S., J. Bone Joint Surg. Am. 89: 780-785, 2007.

FIG. 2B is a line graph showing the annual number of procedures of revision hip replacement surgeries (“THA” line, open circle) and revision knee replacement surgeries (“TKA” line, no circles). This figure originally appeared in Kutz S., J. Bone Joint Surg. Am. 89: 780-785, 2007.

FIG. 3 is an X-ray of a knee joint showing a large defect. Bacteria have been drawn into the defect and indicated with arrows pointing to the bone defect and an overlay showing bacterial contamination in the infection.

FIGS. 4A-4C are images showing a bacterial biofilm. FIG. 4A is a scanning electron microscope (SEM) image of a biofilm. FIG. 4B is a schematic diagram showing a biofilm with the persistor cells at the edge of the film on the blue background. FIG. 4C in a schematic showing the occurrence of a biofilm (olive green shading) with a metal implant surrounded by antibiotic-containing cement.

FIG. 5 is a schematic drawing showing an example of how a non-limiting bone graft of the invention is able to release antibiotic. The polymer coating (green flexible rods) encapsulates the antibiotic (blue triangles) in the graft. In the presence of water, the polymer coating of the implant hydrolyzes and degrades (depicted as green flexible rods stricken through with black, gray, or white bars), thereby releasing the antibiotic (blue triangles) into the surrounding milieu. In this non-limiting embodiment, synthetic bone is shown; however cadaver-sourced allograft bone can also be used.

FIG. 6A is a line graph showing a comparison of the theoretical elution profiles of current bone graft technology (red dotted line) to one of the non-limiting drug-eluting bone implants of the present invention (shown in solid blue line and labeled “ElutiBone”). In this FIG. 6A, the blue solid line refers to the elution profiles of both the generation 2 and the generation 3 fabrications described herein. As shown, the generation 2 and the generation 3 fabrications, two non-limiting drug-eluting bone implant fabrications of the invention, are able to elute drug at a sustained level above the bacteria-killing dose for 6-8 weeks or longer after implantation.

FIG. 6B is a line graph showing the actual elution profile of antibiotic from a non-limiting bone implant of the invention, namely the drug release curve from FIG. 11B (i.e., from the 80 kD PCL in acetone+4% non-water solvent generation 2 fabrication) extended out to twelve weeks (blue line) compared to the rate of bone formation (red line) from time of implantation to 12 weeks post-implantation. As the graph shows, the dips and peaks in bone formation indicate when the osteoclasts and osteoblasts, respectively, are active. Note that the second peak of drug release at 5 week post-implantation occurs shortly before osteoblast recruitment and the peak of bone formation. As FIG. 6B shows, the drug release from the generation 2 fabrication matches the rate of bone growth and remodeling. The generation 3 fabrication is expected to act the same as the generation 2 fabrication shown here. The superimposed image in FIG. 6B is a representation of the host bone integration of the graft shown in larger scale in FIG. 6C.

FIG. 6C is a larger scale of the image from FIG. 6B showing that the implanted graft (the graft shown in FIG. 6C is a generation 3 fabrication) is remodeled into host bone over time. Thus, the implant supports bone growth and healing in addition to preventing infection.

FIG. 7A is a schematic diagram showing a non-limiting fabrication method for making a non-limiting bone implant of the invention. In this embodiment (which may be referred to as the generation 1 fabrication method), an allograft or synthetic bone fragment (i.e., a crouton) is spray-coated with an antibiotic-containing PCL polymer and acetone solution (shown in purple), and then allowed to air dry resulting in a coated bone implant.

FIG. 7B is a schematic diagram showing a non-limiting fabrication method for making a non-limiting bone implant of the invention. In this embodiment (which may be referred to as the generation 2 fabrication method), the allograft or synthetic bone fragments are dip coated in various antibiotic-containing polymer acetone solutions with or without a water non-solvent component (shown in purple) to result in a coated bone implant. In variations of this method, the allograft or synthetic bone fragment may first be soaked in an antibiotic (and, e.g., dried via vacuum drying, heat drying, or air drying) and then dipped into the various antibiotic-containing polymer solution to coat the antibiotic-soaked crouton to result in a coated antibiotic-soaked crouton.

FIG. 7C is a schematic diagram showing a non-limiting fabrication method for making a non-limiting bone implant of the invention. The fabrication method shown in FIG. 7C may be referred to herein as the generation 3 fabrication. This generation 3 fabrication method is used in Example 4 herein. As shown, the products of this fabrication method are multiple bone implants of precise dimensions.

FIG. 8 is a schematic drawing showing additional non-limiting bone implants of the with a description of their distinguishing antibiotic loading written under the image. The distinguishing antibiotic loading character is a product of the fabrication technique and the order in which the polymer coating and antibiotic are applied to the bone graft substrate.

FIGS. 9A-9C are a schematic drawings showing the concept of a bone graft-based drug delivery vehicle encased within a rate-controlling degradable or porous polymer membrane using the generation 2 fabrication method and modifications thereof. The graft porosity provides a high surface-area reservoir to load drug within the pore-filling polymer, and the polymer coating formulation and coating alternatives provide a versatile and tailorable local antibiotic releasing device. Combinations of free drug and microencapsulated drug either or both within the graft pores and/or within the polymer coating allow drug loading and controlled release kinetics versatility to accommodate dosing amounts and release rates.

FIGS. 10A-10D are images showing a number of SEM images of non-limiting allograft bone implants, incorporating a variety of antibiotics; FIG. 10A: Uncoated; FIG. 10B: Tobramycin Sulfate; FIG. 10C: Ciprofloxin HCl; and FIG. 10D: Vancomycin HCl.

FIG. 11A is a line graph showing the percentage of tobramycin released using an in vitro system for non-limiting bone implant formulations (generation 2 fabrications) fabricated with the tobramycin-containing 200 kD polycaprolactone (PCL) polymer coating (dashed line), the tobramycin-containing 80 kD polycaprolactone (PCL) polymer coating (solid line) and the tobramycin-containing 10 kD polycaprolactone (PCL) polymer coating (dotted line).

FIG. 11B is a line graph showing the average tobramycin released at the indicated times from the generation 2 tobramycin-containing 80 kD PCL polymer coated bone implant formulation, with 4% water non-solvent added to the coating formulation and 10% weight of tobramycin.

FIGS. 12A and 12B are diagrams showing the mechanism of the zone of inhibition assay (FIG. 12A) and typical data that may be obtained (FIG. 12B). As shown in FIG. 12A, the zone of inhibition will be greater where there is a higher amount of drug diffusing from the disk onto the lawn of bacteria (thus preventing the bacteria from growing all the way to the disk and killing the bacteria).

FIGS. 13A and 13B are line and bar graphs, respectively, showing the comparison of tobramycin released from different allograft crouton fragments (a type of non-limiting implant generated by the generation 2 fabrication method as described here). FIG. 13A shows the kinetics of drug release, and FIG. 13B shows the zone of inhibition in vitro against E. coli cultures. In FIG. 13A, the solid line with open circles denotes allograft fragments from cohort 1 while the dashed line with closed circles denotes micron-sized allograft particulate matter from cohort 3 (described in Example 1). In FIG. 13B, the solid black bars denote allograft fragment from cohort 1 while the patterned bars denote micro-sized allograft particulate matter from cohort 3 (described in Example 1). Notice the increased bacteriocidal activity of tobramycin released from particulate allograft material as well as lower standard deviations.

FIGS. 14A and 14B are line and bar graphs, respectively, showing the comparison of drug released from coated allograft generation 2 fabrications described in Example 1 (cohort 1: open circles, solid black line in FIG. 14A; solid black bars in FIG. 14B) versus synthetic ProOsteon 500R® generation 2 fabrications described in Example 1 (cohort 2: filled circle, dotted line in FIG. 14A; patterned bars in FIG. 14B) substrates. FIG. 14A shows the kinetics of drug release, and FIG. 13B shows the zone of inhibition in vitro against E. coli cultures.

FIGS. 15A and 15B are line and bar graphs, respectively, showing the comparison of tobramycin release from different coated allograft tobramycin-containing formulations generated using the generation 2 fabrication method, namely cohort 3—PCL (closed circles with dotted line in FIG. 15A and black bars in FIG. 15B) and cohort 4—PCL_{F}/PEG, where F=free tobramycin (open circles with solid line in FIG. 15A and patterned bars in FIG. 15B). Cohorts are as described in Example 1. FIG. 15A shows the kinetics of drug release determined via a 96-well fluorescent assay based on derivatization of tobramycin with o-phthaldehyde (OPA), and FIG. 15B Zone of inhibition in vitro against E. coli cultures. Although there are very few significant differences in the overall effectiveness of the released tobramycin, the kinetics of release demonstrate large differences.

FIGS. 16A and 16B are line and bar graphs, respectively, showing the comparison of release of free (PCL_{F}/PEG—cohort 4: open with solid line in FIG. 16A and black bars in FIG. 16B) versus microencapsulated (PCL/PEG_{M}—cohort 5: closed circles with dotted line in FIG. 16A and patterned bars in FIG. 16B) tobramycin from coated allograft generated according to the generation 2 fabrication method (see FIGS. 9A, 9B, and 9C) with cohorts as described in Example 1 below. FIG. 16A shows the kinetics of drug release, and FIG. 16B shows the zone of inhibition in vitro against E. coli cultures. Only approximately 80% of the tobramycin was released within the 6-week time course; thus, the effective therapeutic release of microencapsulated tobramycin may be extended beyond 6 weeks.

FIGS. 17A, 17B, and 17C are a line graph, a bar graph, and a line graph, respectively, showing the comparison of PCL coating application techniques on tobramycin release from allograft particulate from implants generated according to the generation 2 fabrication method (cohorts are described in Example 1 below). While a slight difference in release kinetics for solvent-cast (PCL/PEG_{M}—cohort 5: closed circles with dotted line in FIG. 17A; black bars in FIG. 17B) versus layer-by-layer solvent cast (PCL_{F}/PEG_{M}/PCL_{F}—cohort 6: open circles with solid line in FIG. 17A; patterned bars in FIG. 17B) are observed (FIG. 17A), there are no statistical differences in the resulting antimicrobial activity as both formulations provide effective antimicrobial activity against E. coli in vitro out to 6 weeks (FIG. 17B). FIG. 17C is the data from FIG. 17A shown at time points closer to the initiation of the study.

FIGS. 18A-18F are scanning electron microscopy images of the surfaces of different implants (also sometimes referred to as allografts or bone grafts) prepared by the dip-coating method (e.g., see FIG. 7B), by dipping natural (allograft) crouton substrates in the formulations produced as indicated, and then drying by vacuum. FIG. 18A shows an uncoated allograft bone implant. FIG. 18B shows an implant made by dipping the allograft crouton in a PCL-containing formulation with 4% water non-solvent in the formulation (top image) and without the water non-solvent component (bottom image). FIGS. 18C-18F show implants made by dipping allograft croutons in a PCL-containing formulation with 4% water non-solvent in the formulation (top image) and without the water non-solvent component (bottom image) containing ciprofloxin (FIG. 18C), rifampicin (FIG. 18D), oxacillin (FIG. 18E), and vancomycin (FIG. 18F).

FIGS. 19A, 20A, and 21A are line graphs showing the kinetic release of antibiotics from non-limiting bone implants formulated with (red diamonds) or without (blue squares) a 4% water non-solvent for implants coated in a formulation containing ciprofloxin (FIG. 19A), rifampicin (FIG. 20A), and vancomycin (FIG. 21A).

FIGS. 19B, 20B, and 21B are bar graphs showing the results of zone of inhibition bioactivity data studies for implants formulated with (red bars) or without (blue bars) a 4% water non-solvent for implants coated a formulation containing ciprofloxin (FIG. 19B), rifampicin (FIG. 20B), and vancomycin (FIG. 21B).

FIGS. 22A and 22B are photographs of mice implanted with a non-limiting bone implant coated with a PCTL tobramycin solution (FIG. 22A) or an uncoated allograft implant generated without drug (FIG. 22B). These implants were all generation 2 fabrications made using particulate allograft. Representative host individuals are depicted.

FIGS. 23 and 24 are bar graphs showing the appearance (FIG. 23) and behavior (FIG. 24) of mice implanted with a non-limiting bone implant of the invention that was uncoated (purple bars) or with a non-limiting bone implant of the invention that was coated with an antibiotic-containing polymer coat (red bars) over the indicted period of days post-implantation, where the higher the score, the less natural the animal appeared and behaved.

FIGS. 25A and 25B are photographs showing non-limiting examples of slide molds (FIG. 25A) and silicone isolators (FIG. 25B) that can be used to generate the generation 3 fabrication bone implants described herein.

FIG. 26 is a schematic diagram showing a non-limiting method for quantitating the amount of antibiotic drug present in the coating on a non-limiting bone implant of the invention.

FIG. 27 is a line graph showing the results of compression testing of generation 3 fabrication bone implants including or not including drug.

FIG. 28 is a series of photographs showing scanning electron microscope (SEM) images of generation 3 fabrications stored at the indicated temperatures for the indicated length of time.

FIG. 29 is a bar graph showing the results of mechanical testing of generation 3 fabrication bone implants, measuring strength (left panel) and modulus (right panel).

FIG. 30 is a bar graph showing the zones of inhibition of bone filler comprising 95% PCL:5% PEG (blue bars), 98% PCL:2% PEG (red bars) and 100% PCL (green bars) at the indicated weeks post-implantation.

FIG. 31 is a line graph showing the zones of inhibition of bone filler comprising 95% PCL:5% PEG with 10% weight/weight tobramycin (blue diamond), 98% PCL:2% PEG with 10% weight/weight tobramycin (black squares) and 100% PCL with 10% weight/weight tobramycin (green triangles) at the indicated weeks post-implantation.

FIG. 32 is a line graph showing the difference in the ability of an antibiotic-containing bone implant (solid line) and a no drug polymer coated implant (dotted line) to kill 10⁹ CFU S. aureus bacteria in vitro.

FIGS. 33A-33C are photographs of cultured osteoblasts exposed to nothing (FIG. 33A), a tobramycin-soaked ProOsteon fragment (FIG. 33B) or a generation 3 fabrication (FIG. 33C) that includes 10% tobramycin.

FIGS. 34A and 34B are photographs showing the timeline of the implantation studies in terms of animal work (FIG. 34A) and histology work (FIG. 34B). FIG. 34C is an expanded view of a photograph taken from FIG. 34A showing the critical size of the radial defect in the rabbit in situ.

FIG. 35 is a line graph showing the survivability percentage of animals implanted with a standard implant without infection (blue diamonds), a standard implant with infection of 10⁵ CFU S. aureus bacteria (red squares), and a generation 3 fabrication (molten-cast croutons made according to the method depicted in FIG. 7C and then dipped in a PCL acetone solution (60 mg/ml) with infection of 10⁷ CFU S. aureus bacteria (green circles). The generation 3 fabrication implant was a molten-cast crouton made according to the method depicted in FIG. 7C, and then the crouton was dip coated in a PCL solution (60 mg/ml) with no drug in the coating. As shown in this FIG. 35, the generation 3 fabrication implant with a high dosage of bacterial infection (green circles) showed as much survivability (100%) as the prior art implant with no infection (blue diamonds).

FIG. 36 is a line graph representing a read-out from an HPLC analysis of rabbit urine taken pre-surgery (blue line), one week post-implantation surgery (red line), 2 weeks post-surgery, 3 weeks post-surgery (cyan), and 4 week post-surgery (purple) from rabbits implanted with a generation 3 fabrication comprising 90% PCL: 10% PEG and tobramycin in a uniform mixture. The green line is a positive control showing the read out of an HPLC analysis of rabbit urine taken pre-surgery that was spiked with tobramycin to confirm that this HPLC analysis could detect tobramycin in the urine. This FIG. 36 shows that the local release of tobramycin from the implant does not affect the rabbit systemically because it does not appear in the rabbit's urine.

FIG. 37 are a series of X-ray images taken from of the radial bones of rabbits implanted with an implant with no polymer coating and no antibiotic and infected with 10⁵ CFU S. aureus (top row of images), animals implanted with an implant with no polymer coating and no antibiotic but not infected with any bacteria (middle row of images) and animals implanted with the generation 3 fabrication (i.e., having a uniform mixture of bone (synthetic or allograft)/polymer/drug) and infected with 10⁷ CFU S. aureus (bottom row of images) before surgery (left column), immediately post-surgery (2^{nd} column from the left), 2 weeks post-surgery (3^{rd} column from the left) and 8 weeks post-surgery (right column).

FIGS. 38A, 38B, and 38C are a bar graph (FIG. 38A) and X-ray images (FIGS. 38B and 38C). FIG. 38A shows the size of the implant at the indicated times post-surgery, where the implant decreases in size over time, as is expected as the implant is replaced by the host's own bone. FIGS. 38B and 38C show the implant at 10 weeks and 24 weeks post-surgery, respectively. As shown in FIG. 38B, the graft (boxed in red) is surrounded by a halo (in yellow), where the host's cells are actively re-absorbing the implant graft.

FIG. 39 is a series of photographs showing callus formation in animals implanted with an antibiotic-containing bone implant.

FIGS. 40A-40D are photographs showing images of generation 3 implants taken in situ from a cohort 2 animal (ProOsteon only with infection; FIG. 40A), a cohort 4 animal (ProOsteon coated with polymer and no drug with infection; FIG. 40B), an animal with normal bone (FIG. 40C), and a cohort 7 animal (ElutiBone; FIG. 40D). Cohorts are described in Example 6

FIGS. 41A and 42B are a line graphs showing the differences in infection score (FIG. 41A) and osseoinhibition score (FIG. 41B) of animals implanted with the antibiotic-containing generation 3 fabrication (blue diamonds), bone filler only (red squares), and no antibiotic-containing polymer coated implant (yellow triangles). Above FIGS. 41A and 41B are images showing how the infection and the osseoinhibition were scored.

FIG. 42 is a bar graph showing the reduced infection in tissue and bone in animals implanted with an antibiotic-containing generation 3 fabrication (“ElutiBone cohort”) as compared to animals implanted with ProOsteon fragments cut to be 2 mm×2 mm×6 mm (“Non-ElutiBone (Infection)”).

FIG. 43 is a photograph of a gram stain of a soft tissue histological sample taken from a cohort 4 animal.

FIG. 44 is a photograph of a gram stained bone slide taken from a cohort 2 animal.

FIGS. 45A-45C are diagrams showing three non-limiting different applications for the implants described herein, where the implant is used in combination with a prosthetic as an applied filler adjunct to implant placement or as an on-board pre-applied degradable drug-releasing device on the prosthesis.

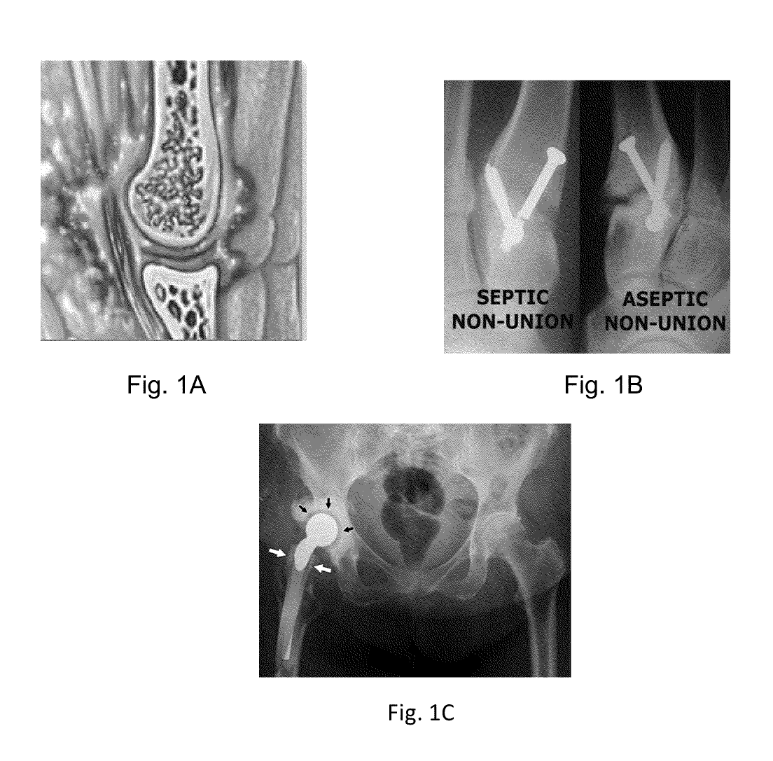

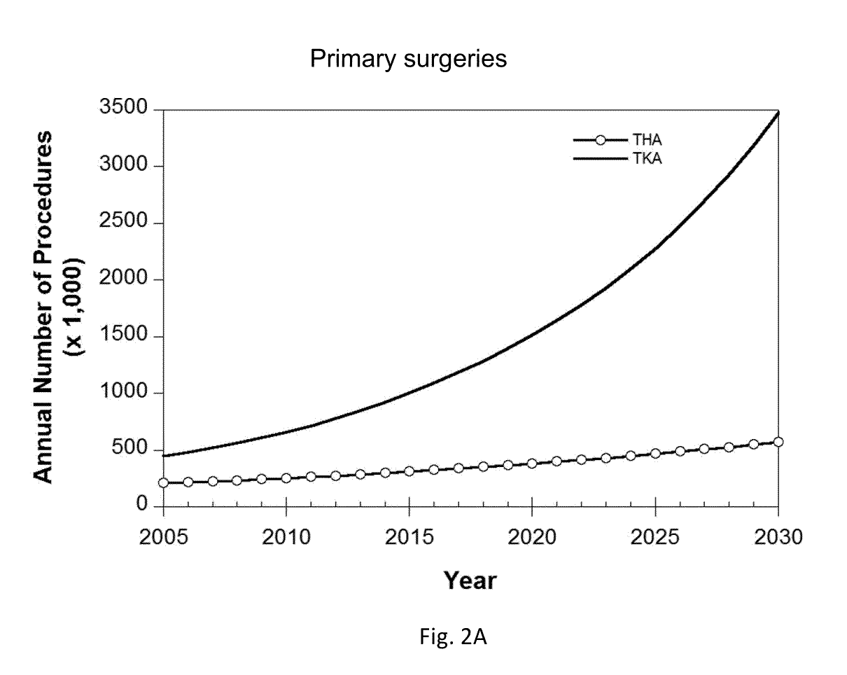

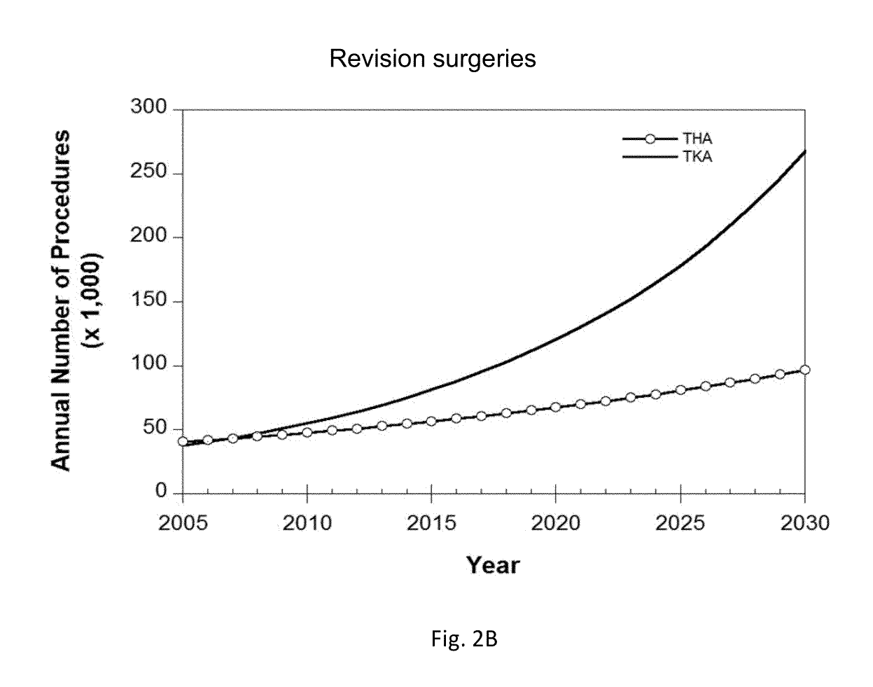

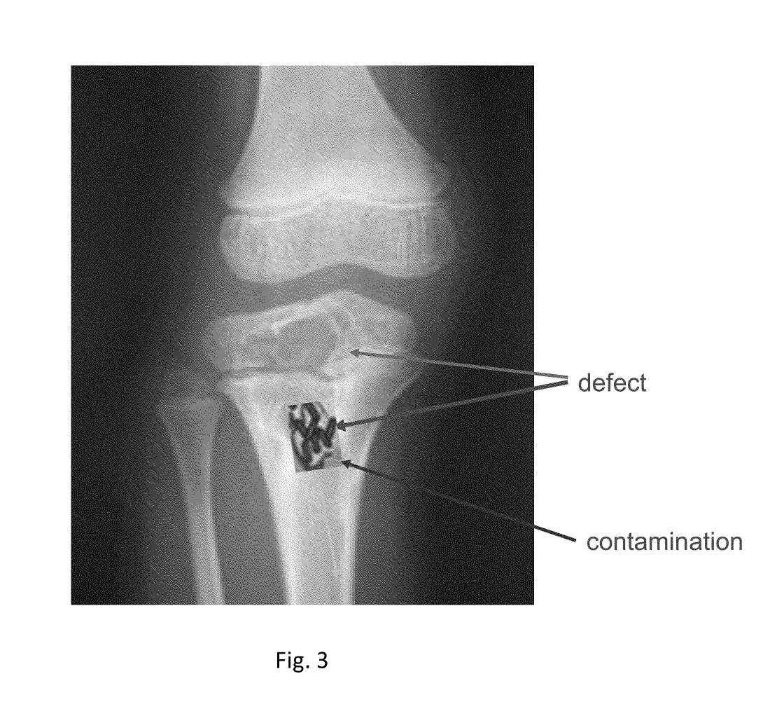

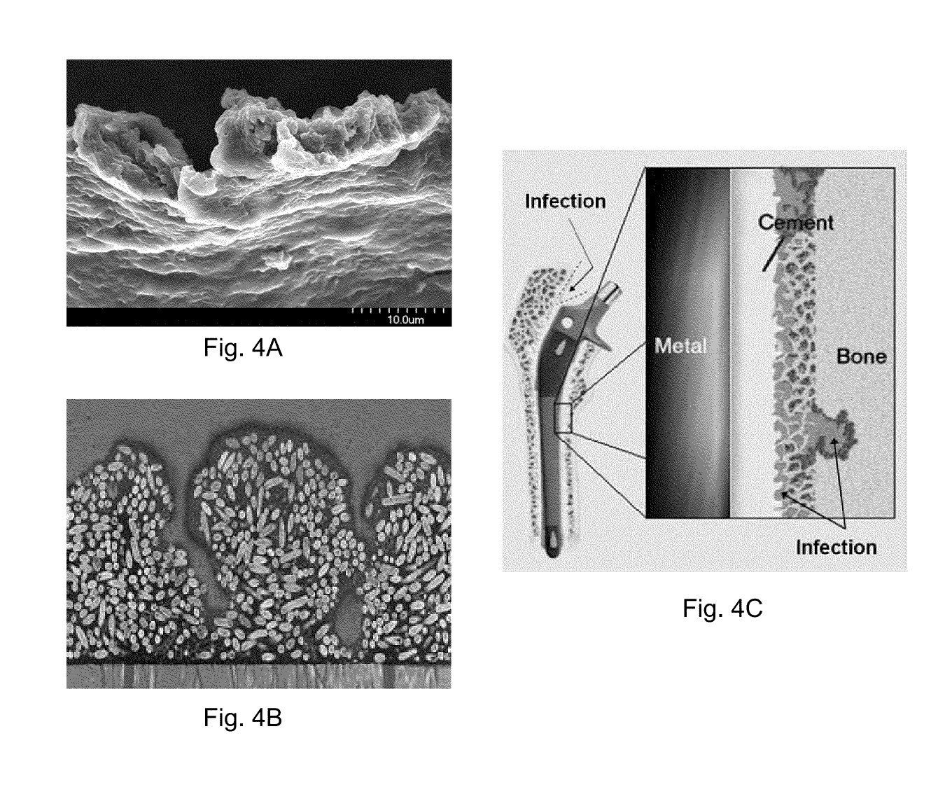

### DETAILED DESCRIPTION OF SPECIFIC EMBODIMENTS

The present invention is based upon the development of methods and systems for the long-term treatment of orthopedic injuries and conditions.

The published patents, patent applications, websites, company names, and scientific literature referred to herein establish the knowledge that is available to those with skill in the art and are hereby incorporated by reference in their entirety to the same extent as if each was specifically and individually indicated to be incorporated by reference. Any conflict between any reference cited herein and the specific teachings of this specification shall be resolved in favor of the latter.

Definitions. As used in this description and the accompanying claims, the following terms shall have the meanings indicated, unless the context otherwise requires:

By “therapeutic level” is meant a level of a drug required to have a therapeutic effect. For example, if the drug is a growth factor, a therapeutic level of that drug is a level required to enact growth by a cell expressing the receptor to the growth factor. If the drug is an antibiotic, a therapeutic level of that drug is a level required to inhibit and/or prevent growth of a pathogen susceptible (i.e., responsive) to that antibiotic. In some embodiments, the therapeutic level is maintained at the implantation site. In other words, the therapeutic level is maintained throughout the implant itself and within an area of at least 1 centimeter, or at least 2 centimeters, or at least 5 centimeters, or at least 7 centimeters, or at least 10 centimeters, or at least 12 centimeters, or at least 15 centimeters, or at least 20 centimeters from the outside edge of the implanted implant.

By “bolus” is meant level of diffusion from the implant that is greater than a therapeutic level of diffusion. A bolus can sustain its level of diffusion for at least 24 hours, or at least 48 hours, or at least 72 hours following initiation of the bolus, after which the level of diffusion returns to a therapeutic level.

By “implant” is meant an object that can be or has already been implanted into a vertebrate host for medical or therapeutic purposes (including, for example, experimental medical or therapeutic purposes). In some embodiments, the vertebrate host animal is one in need of an implant (e.g., a patient). In some embodiments, the implant is a solid that may be carved to fit a vertebrate animal in need of the implant prior to implantation. For example, if the vertebrate animal is a rabbit and the implant is to treat a damaged radial bone of the rabbit, the implant may start as a 2 mm×2 mm×6 mm solid block that then can be carved (e.g., during the surgery by a medical or veterinary practitioner) to fit the defect precisely prior to implantation. Likewise, if the vertebrate animal is a human and the implant is designed to fill vacant space resulting from removal of an infected total knee arthroplasty and re-insertion of a new knee prostheses, the implant may start as a 1 cm×1 cm×4 cm solid block and then may be carved by the surgical team during the surgery to fit the vacant space. In some embodiments, the implant may be an injectable paste. In some embodiments, the injectable paste may harden in the host following implantation.

As used herein, by “vertebrate host” or “vertebrate animal” is meant any animal that has a backbone. Included as vertebrate hosts are amphibians, reptiles, birds, fish, mammals, and any other type of animal that has a backbone. In some embodiments, the vertebrate host is a mammal including, without limitation, a domesticated animal (e.g., cow, sheep, goat, llama, horse, donkey, pig, camel, ostrich, chicken, emu, etc.), a laboratory animal (e.g., a chimpanzee, a baboon, a rabbit, a rat, a mouse, a hamster, etc.), a pet animal (e.g., cat, dog, parrot, etc.), an endangered animal (e.g., polar bear, tiger, lion, elephant, rhinoceros, blue whale, hippopotamus, etc.), and a human.

By “curing” is meant the toughening or hardening of a polymer-containing material by cross-linking polymer chains. Curing can be accomplished by a number of methods including, without limitation, the addition of a chemical, ultraviolet radiation, electronic beam, or heat. In some embodiments, a solid implant is solidified by curing through the addition of heat a uniform mixture comprising a degradable polymer, bone, and a drug.

By “uniform mixture” is meant a mixture comprising two or more components, where the components are in approximately the same ratio to one another throughout the mixture, when considered at a macro level, even if at a molecular level the distribution of components is not even. For example, if the mixture is a solid block, a full thickness chip taken out of the solid will have approximately the same ratio of components as the larger block. In another example, a centimeter-sized chip removed from the bulk solid will have approximately the same ratio of components as the bulk solid. Likewise, if the mixture is in a liquid state, an aliquot of the mixture will have approximately the same ratio of components as the larger volume from which the aliquot was taken. In some embodiments, where the mixture is in a liquid state, the uniform mixture does not have to be stirred or agitated to maintain the uniformity of its components in the aliquot. In some embodiments, where the mixture is in a liquid state, the uniform mixture must be stirred or agitated for at least one minute prior to taking an aliquot to maintain the uniformity of its components in the aliquot.

By “degradable” is meant that the structure of a polymer can be broken down by hydrolysis and/or by the host's cells or enzymes following implantation of the implant into the host.

By “bone” or “bone graft” is meant synthetic bone or natural bone collected from living vertebrate animals or cadavers. The synthetic bone may be fabricated or synthetic by man, or may be obtained commercially (e.g., the ProOsteon synthetic bone). Natural bone may be collected or harvested from living vertebrate animals, or collected or harvested from cadavers (e.g., cadaver-derived bone, which is allogeneic to the recipient of the implant). In some embodiments, the synthetic bone or natural bone is fragmented or pulverized into micron-sized particulates. In some embodiments, the bone is sterilized (e.g., in an autoclave). In some embodiments, the bone or bone graft is intended to be used clinically as a replacement filler to fill defects and allow production (e.g., by providing a scaffold) of new autologous bone by the host receiving the implant. No MHC-expressing cells or antigen are included in bone (e.g., processed bone). Synthetic bone solids can be readily directly and routinely synthesized from calcium or strontium-based precursors in large batches in commercial ovens, and pulverized (i.e., ground) into pieces and granules, sterilized and certified to be clinical grade filler biomaterial.

By “drug” is meant any type of molecule, or a mixture or complex of molecules, that may be administered to a host with the intention of that molecule or mixture having a therapeutic effect on that host. A therapeutic effect may be a stimulatory effect on autologous or allograft cells (e.g., stimulating growth of cells that repair wounds) or an inhibitory effect on pathogenic cells or agents (e.g., inhibiting growth of bacteria or viruses). Thus, a drug shall include, without limitation, an antibiotic, a growth factor, a vasodilator, a vasoconstrictor, an angiogenesis factor, a chemotactic factor, a cytokine, a pharmaceutical small molecule, a pharmaceutical biological, an enzyme, an antibody, or a mixture or two or more of the preceding. In some embodiments, the drug is water-soluble.

In some embodiments, the drug is thermostable. By “thermostable” is meant that the drug's activity after heating the drug for at least one minute to a temperature higher than 37° C. is at least 80% or 85% or 90% or 95% or 99% of the activity of that drug at 37° C. For example, a thermostable drug is one that has an activity of at least 80% or 85% or 90% or 95% or 99% of a the activity of the drug at 37° C. when the drug is heated for at least one minute or at least two minutes or at least five minutes to a temperature that is at least 55° C. at least 60° C. or at least 65° C. or at least 70° C. or at least 75° C. or at least 80° C. or at least 85° C. or at least 90° C. or at least 95° C., or at least 98° C., or at boiling point, or at the thermal processing point used for drug processing in the graft filler. Some drugs are thermostable (e.g., the thermostable antibiotics described below). For example, thermostable drugs for this intent include, without limitation, tobramycin, gentamicin, vancomycin, and the cephalosporins.

In addition, methods are known for making almost any protein thermostable (see, e.g., Chautard et al., Nature Methods 4(11): 919-921, 2007; Hoseki et al., J. Biochem. 126(5): 951-956, 1999; Liao et al., Proc. Natl. Acad. Sci. USA 83(3): 576-580, 1986; Iwamoto et al., Appl. Environ. Microbiol. 73(17): 5676-5678, 2007).

In some embodiments, the drug is selected based on the need of the host. For example, for periprosthetic infections following an implant, many involve pathogens such as gram positive organisms such as Staphylococcus aureus and Staphylococcus epidermidis, both of which are inhibited by tobramycin. Likewise, gentamicin (another antibiotic) will inhibit E. coli Enteroacteriaeceae and Pseudomonas aeruginosa. Additional antibiotics can be used to address antibiotic resistant strains of these bacteria. For example, vanomycin can inhibit methicillin-resistant Staphylococcus aureus (see Cui et al., J. of Bone and Joint Surgery 89(4): 871-882, 2007).

Despite clinical, material, and pharmaceutical advances, infection remains a major obstacle in orthopedic surgeries. Successful solutions must extend beyond bulk biomaterial and device modifications, integrating locally delivered pharmaceuticals and physiological cues at the implant site, or within large bone defects with prominent avascular spaces. In some embodiments, the invention provides an approach involving coating clinically familiar allograft bone with an antibiotic-releasing rate-controlling polymer membrane for use as a matrix for local drug release in bone in the context of clinical bone graft fillers used in bone-filling and wound space filling functions. The kinetics of drug release from this system can be tailored via alterations in the substrate or the polymeric coating. Drug-loaded degradable polymer (e.g., polycaprolactone and its copolymers) coatings releases bioactive tobramycin from both cadaveric-sourced cancellous allograft fragments and synthetic hybrid coralline or other ceramic bone graft fragments with similar kinetics over a clinically-relevant 6-week timeframe. However the micron-sized allograft particulate provides extended bioactive tobramycin release. Surprisingly, the addition of a GRAS water-soluble polymer (e.g., polyethylene glycol as an example coating poragen) in different amounts to the graft coating formulation dramatically changes tobramycin release kinetics without a significant impact on released antibiotic bioactivity. Incorporation of pre-formulated lipid-microencapsulated tobramycin into the polymer coating did not significantly modify tobramycin release kinetics. In addition to releasing bactericidal concentrations of tobramycin, antibiotic-loaded allograft bone provides recognized beneficial osteoconductive potential, encouraging bone in-growth and tissue neogenesis, possibly decreasing orthopedic surgical infections with improved filling of dead space and new bone formation.

However, despite significant multi-disciplinary clinical innovations combined with biomaterial and pharmaceutical approaches, including now-standard systemic antibiotic prophylaxis and new bone grafting biomaterials, infection remains a major complication in total joint revision surgery, with rates ranging from 8-15% and relapsing infection representing a significant threat (20-30%) to wound healing (Conterno and da Silva Filho, Cochrane Database Syst Rev, (2009) CD004439; Landersdorfer et al., Clin Pharmacokinet, 48 (2009) 89-124). This represents a considerable healthcare burden as the demand for total joint replacements continues to rise with the aging population. The number of post-arthroplasty infectious complications is projected to increase from current levels of 17,000 cases to an overwhelming 266,000 cases annually by 2030 (Bernthal et al., PLoS One, 5 (2010) e12580.

Despite clinical routine of filling avascular dead spaces with bone graft filler materials and bone cements and prophylactic systemic antibiotics, infection remains the second most prevalent complication associated with orthopedic surgeries. The most common infection is osteomyelitis (Aronin et al., Biomaterials, vol. 31, pp. 6417-6424), acutely caused by perioperative introduction of pervasive pathogens such as staphylococcus (Roald et al., Blood Coagul. Fibrinolysis, vol. 5, pp. 355-63, 1994) into the avascular spaces surrounding the orthopedic graft. In fact, Stapholococcal strains account for more than 90% of osteomyelitis cases (Gogia et al., Semin Plast Surg, vol. 23, pp. 100-7, 2009).

In some cases, the high infection rate may be due to antibiotic resistance that develops from interactions between the environmental conditions, natural selection pressures, and antibiotic misuse (Peters et al., J Infect Dis, vol. 197, pp. 1087-93, 2008). The emergence of antimicrobial resistance based on natural selective pressures and complicated by clinical antibiotic overuse is typified by the increasing number of impotent antibiotics due to the widespread use of single systemic antibiotic therapy. Subsequently, local antibiotic combination therapy may serve to alleviate antibiotic resistance concerns. Combination therapy aims to use multiple antibiotics that produce the analogous or even enhanced therapeutic effects with lower doses of each antibiotic. The utilization of combination therapy has proven helpful in clinical practice when treating chronic staphylococcal infections and the subsequent decreased antibiotic susceptibility of the infection (Bernard et al., J Antimicrob Chemother, vol. 53, pp. 127-9, 2004). In addition to widespread systemic single antibiotic overuse, local antibiotic delivery can also spur antibiotic resistance. Antibiotic concentrations below the therapeutic dose, such as that observed with commercially available drug releasing bone cement (PMMA) and allograft bone soaked in antibiotic solutions in the surgical theater (off-label), inadvertently promote drug-resistance in bacteria (Diefenbeck et al., Injury, vol. 37 Suppl 2, pp. S95-104, 2006). “Off label” preparations such as this are unpredictable, lacking quality controls that may inadvertently promote antibiotic-resistance (Kanellakopoulou and Giamarellos-Bourboulis, Drugs, vol. 59, pp. 1223-32, 2000; Ayers, N., J Polym Sci A Polym Chem, vol. 46, pp. 7713, 2008). Indeed, current methods of simple physical absorption of drug to allograft bone can result in only a bolus release (Kanellakopoulou and Giamarellos-Bourboulis, supra), with no sustained release to combat further infections. Polymer-controlled antibiotic release from allograft bone material is a desirable alternative as it allows for long-term antibiotic success by tailoring antibiotic dose, combination drug therapies, control of drug dose release kinetics and local drug delivery from an osteoconductive clinically approved bone filler biomaterial device.

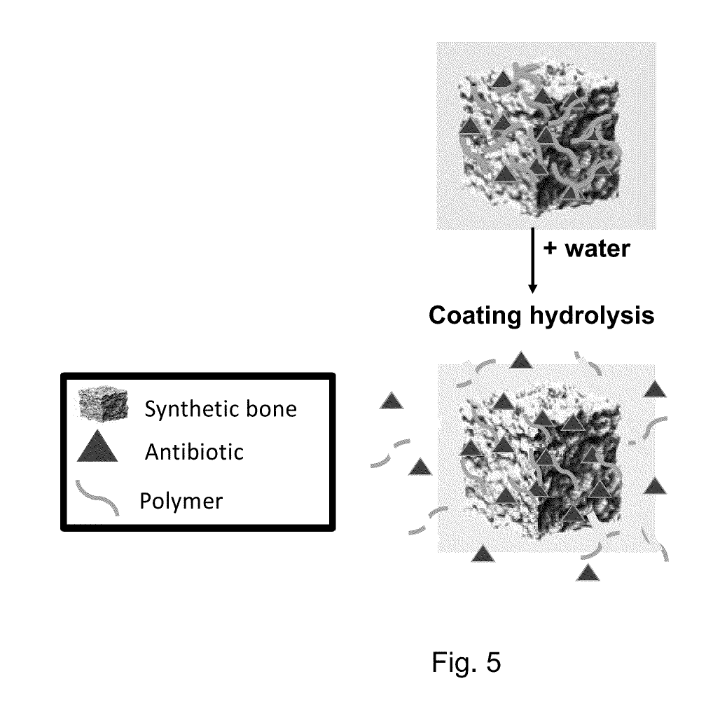

In combating infection, an antibiotic administered with a bone implant will, in some embodiments of the present invention, hit the “sweet spot” in combating initial infection, while continuing to elute the antibiotic for a prolonged period of time (e.g., 6-8 weeks post-implantation, or even up to 10 weeks or longer post-implantation). FIG. 5 is a schematic graph comparing current technologies to the some non-limiting embodiments of the implant (termed “ElutiBone”) of the present invention. In some embodiments, the implant of the present invention will maintain drug above the minimum inhibitory concentration to prevent the formation of antibiotic resistance (see FIG. 5).

To overcome this problem, in some embodiments, the invention described herein resulted from efforts to investigate the beneficial effect of drug/polymer and drug/physiological fluid solubility and miscibility on drug-release profile, water-soluble antibiotics (e.g., vancomycin or oxacillin) and non-water soluble antibiotics (e.g., ciprofloxacin or rifampicin) were released from a polymer-loaded controlled releasing membrane with and without the incorporation of polymer non-solvent (e.g., water) into the device formulation. Moreover, the danger of multi-drug resistant pathogens will be investigated by considering the combinatorial therapeutic efficacy of other clinically important antibiotics (e.g., ciprofloxacin, rifampicin, and vancomycin) against S. aureus as released from a local drug delivery system using a polymer-controlled releasing membrane coated onto allograft bone. This can be studied as two different graft filler materials formulated each with a single antibiotic and controlling polymer membrane, and mixed in the wound site in varying proportions, or alternatively by combining more than one drug together into a single bone graft filler within a polymer rate-controlling membrane. Tailoring the release kinetics as a function of antibiotic solubility should provide clinicians with a long-term, antibiotic delivery system that can be customized and combined to fit each patient's needs while concurrently mitigating the development of antibiotic resistance.

By developing a local drug delivery system that controls the release of antibiotic via a degradable polymer (e.g., polycaprolactone (PCL) and its copolymers) membrane coated on an implantable allograft bone delivery vehicle, an increase in the bioactive longevity of antibiotic therapy is anticipated (Roald et al., Blood Coagul. Fibrinolysis, vol. 5, pp. 355-63, 1994). Furthermore, combinatorial antibiotic controlled release formulations on these bone graft fillers prepared for each antibiotic will provide clinicians with an “a la carte” method for customizing antibiotic treatment that can be tailored to meet each patient's needs while mitigating the development of bacterial resistance due to systemic and prolonged antibiotic overuse and poor patient compliance.

Autograft bone, or patient-harvested bone, is the gold standard for bone grafting, providing a highly compatible, bioactive, structural matrix as the basis for wound healing. However, cellular death during transplantation, inadequate sourcing due to other pathologies, harvest site morbidity, pain, and cosmetic disfigurement, culminate in a substantial 8.5-20% complication risk, including acute and chronic or recurring infection (Nandi et al., Indian J Med Res, vol. 132, pp. 15-30, 2010; Kundu et al., J Mater Sci Mater Med, vol. 21, pp. 2955-69, 2010; Aronin et al., Biomaterials, vol. 31, pp. 6417-24). Thus, allograft or cadaveric-sourced bone tissue has become an increasingly popular defect and wound packing material, increasing 15-fold over the past decade to now account for almost a third of the over 500,000 orthopedic graft procedures performed annually in the United States to treat traumatic or other boney defects (Aronin et al., supra; Kanellakopoulou and E. J. Giamarellos-Bourboulis, supra). Importantly, allograft bone is processed to remove all cellular and proteinaceous components, leaving only the osteoconductive, and to a more limited extent, osteoinductive mineral component of the graft to provide a structural template for orthopedic repair, and promote integration and turnover by the patient's natural osteoclast and osteoblast populations.

Similarly, synthetic bone (e.g., comprising calcium and/or strontium based ceramic filler biomaterials) also promotes integration and turnover by the patient's natural osteoclast and osteoblast populations. On such synthetic bone is the commercially available ProOsteon substrate (available from, for example, Biomet, Inc., Warsaw, Ind.).

Successful solutions to implant-centered infection might best integrate local, rate-controlled drug delivery with appropriate wound and defect filler materials, particularly for implants and large bone defects with prominent avascular spaces or where penetration from systemic antibiotic administration is compromised (Landersdorfer et al., Clin Pharmacokinet, 48 (2009) 89-124; C. Ketonis et al., Tissue Eng Part A, 16 (2010) 2041-2049; C. Ketonis et al., Clin Orthop Relat Res, 468 (2010) 2113-2121; C. Ketonis et al., Antimicrob Agents Chemother, 55 (2011) 487-494; C. Ketonis et al., Bone, 48 (2011) 631-638; N. M. Mathijssen et al., BMC Musculoskelet Disord, 11 (2010) 96).

Treating bone infections is intrinsically complicated by poor bioavailability and drug pharmacokinetics in bone that limit efficacy of systemically administered antibiotic therapy. Bone vascular physiology enables a niche for diverse types of opportunistic pathogens introduced at the time of injury, intraoperatively, or later by hematogenous sourcing to produce difficult-to-treat infections. Antibiotic penetration into the bone as well as the limited vasculature of the affected bone must be considered when designing a clinical treatment strategy (Landersdorfer et al., Clin Pharmacokinet, 48 (2009): 89-124; Chen et al., Arch Orthop Trauma Surg, 125 (2005): 369-375). Although systemic intravenous antibiotics are often sufficient in combating these opportunistic pathogens, the negative impact of a standard 4-6 week course of antibiotics cannot be neglected. Inappropriate use of antibiotic therapies, such as poor selection, inadequate dosing, broad-spectrum antibiotic overuse, and poor patient therapy follow-through, have all accelerated pressure towards multi-drug resistant microbes. The CDC reports an alarming rise in the antibiotic resistance of the major pathogen, Staphylococcus aureus, to at least one of the most common antibiotics from 2% in 1972 to 63% by 2004 (MRSA vs. MSSA) (see Office of the Associate Director for Communication, 2006, http://www.cdc.gov/media/pressrel/r061019.htm?s_cid=mediarel_r061019_x, accessed Jun. 23, 2011). Furthermore, some systemically administered antibiotics may not achieve therapeutic levels in bone, inadvertently supporting the development of resistance. Therefore, options for local sustained antimicrobial therapies are increasingly attractive. A local drug delivery mechanism overcomes bioavailability and systemic delivery issues, limits development of systemic antibiotic resistance while delivering sustained amounts of drug sufficient to both resist and eliminate microbial infection beyond an acute time course (Patzakis and Zalavras, J Am Acad Orthop Surg, 13 (2005): 417-427). Local delivery of antibiotics offers effective killing using higher doses (up to 1000-fold greater than systemically delivered (Costerton, J. W., Rev Infect Dis, 6 (1984): 608-616; Diefenbeck et al., Injury, 37 Suppl 2 (2006): S95-104) precisely at the site of infection while avoiding systemic toxicity associated with high doses (Diefenbeck et al., Injury, 37 Suppl 2 (2006): S95-104). Unfortunately, many approaches to achieve local antibiotic release from bone grafts with desirable therapeutic kinetics—either actively or passively—are often characterized by an early bolus release and subsequent slow leaching of antibiotic at sub-therapeutic levels that may also promote antibiotic-resistance (Kanellakopoulou and Giamarellos-Bourboulis, Drugs, 59 (2000): 1223-1232; Diefenbeck et al., Injury, 37 Suppl 2 (2006): S95-104). Thus, improved control over local drug release in terms of dose control and duration is likely a necessity for efficacious long-term delivery and antimicrobial efficacy.

Drug delivery directly to bone in general and also to avascular traumatized or infected bone presents a pharmaceutical and pharmacokinetic challenge. Currently, bone grafts are used for musculoskeletal mechanical support as well as space filling and osteoconductive foundation for new bone deposition and healing. Incorporating a space filling material with the controlled degradation of a synthetic polymer may provide features appropriate for prophylactic controlled drug delivery. Importantly, most synthetic polymers alone are inappropriate as bulk materials for orthopedic needs that may require mechanical integrity for up to a year or more and also bone regeneration/healing induction. As a clinically recognized biomaterial, resorbable polycaprolactone (PCL) and its copolymers may exhibit the requisite enhanced temporary structural functionality sufficient for bone implant use while also providing appropriate characteristics for rate-controlled drug delivery and degradability (Lowry et al., J Biomed Mater Res, 36 (1997): 536-541; Coombes et al., Biomaterials, 25 (2004): 315-325; H. I. Chang et al., J Control Release, 110 (2006): 414-421). Thus, with PCL's precedent use in bone implants, PCL and its copolymers may offer a significant opportunity to endow clinically familiar bone graft filler materials with an antibiotic-releasing, rate-controlling coating for extended drug delivery.

The controlled hydrolytic degradation of PCL (Lam et al., Biomed Mater, 3 (2008): 034108; Hutmacher et al, J Tissue Eng Regen Med, 1 (2007): 245-260) offers a versatile range of times for extended release kinetics under certain physiological circumstances in tissues. However, a mixed multi-polymer barrier might be more appropriate (Wei et al., Int. J. Pharm, 381 (2009): 1-18). In this regard, polyethylene glycol (PEG), a common biomaterial generally regarded as safe by the FDA may be incorporated into the PCL (co)polymer coating formulation as a poragen and to improve drug loading and solubility (i.e., for certain poorly water-soluble antibiotics also not miscible with PCL and its copolymers), but may also provide more versatile release kinetics for different dosings or applications. Thus, antibiotic loading and subsequent release kinetics might be adjusted and tailored via the rate-controlling polymer coat formulation.

PCT Publication No. WO 2011/127149 (from International Application No. PCT/US2011/031394), and US Patent Publication No. 2009/0324683; (both of which are hereby incorporated by reference in their entireties) describe

The concept of some of the non-limiting bone graft implants of the invention is depicted schematically in FIG. 5. The finished bone implant is shown schematically at top of FIG. 5. As shown in FIG. 5, in the presence of water (e.g., in situ), the polymer shell will degrade and coating hydrolysis occurs, degrading the polymer (depicted as green curves crossed out with a black, white, or grey line to indicate degradation) thus freeing the antibiotic drug (depicted as blue triangles). The rate of release of the antibiotic drug is controlled by the thickness of the polymer coating, the molecular weight of the polymer, and the type of antibiotic drug incorporated into the implant.

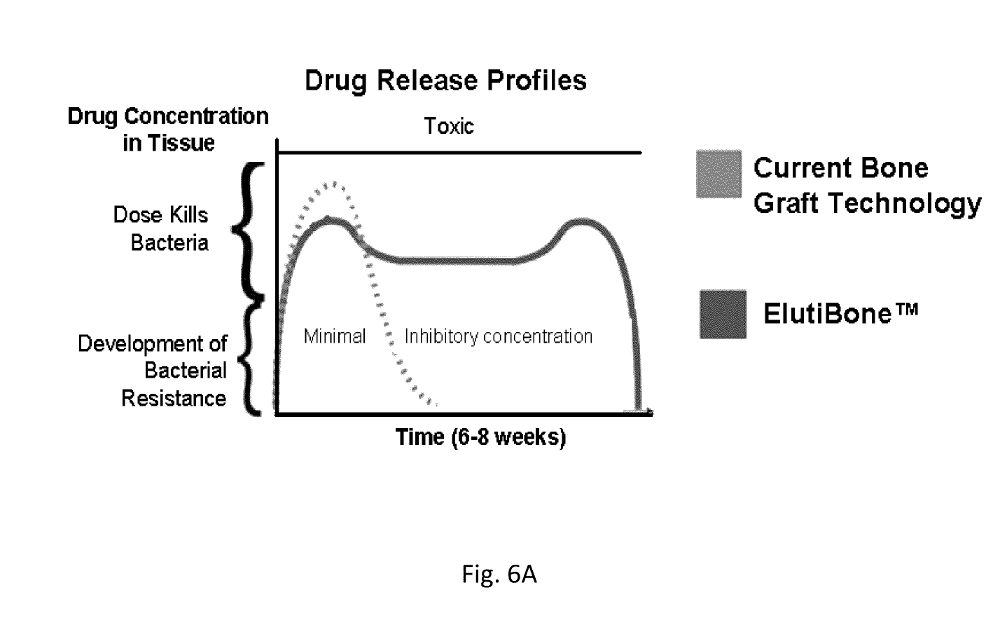

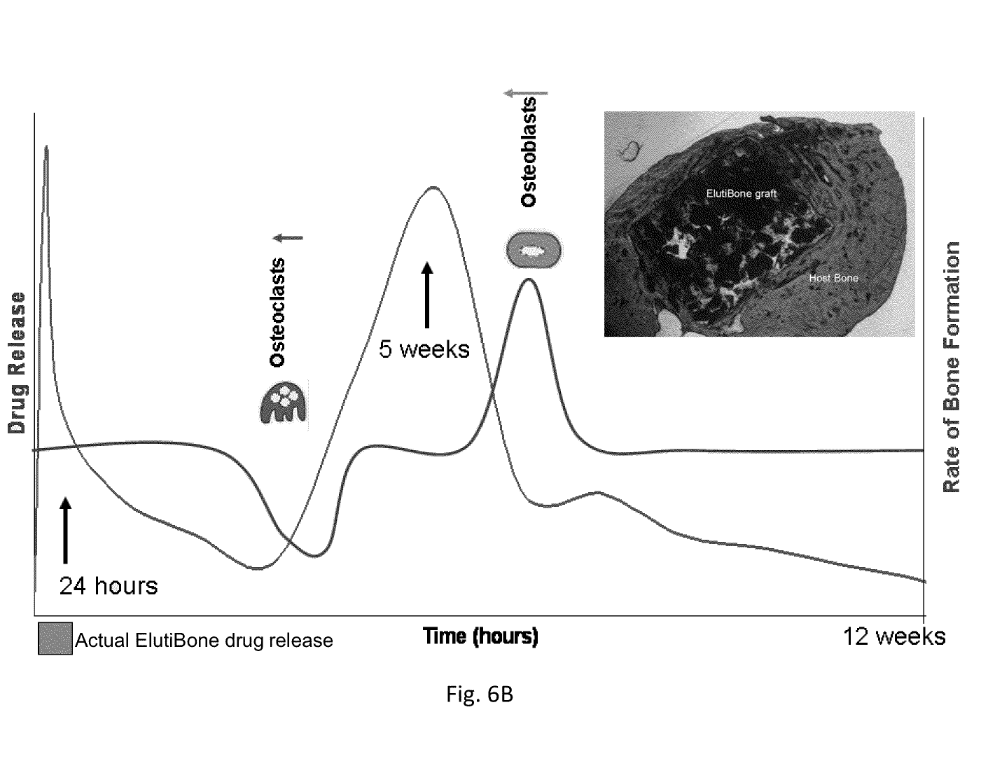

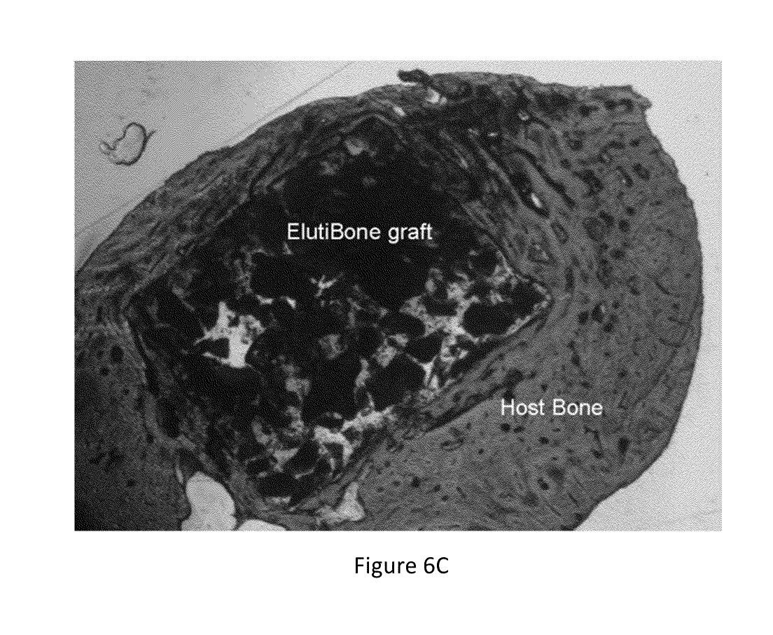

The graph depicted in FIG. 6A shows a comparison of the drug release profile of current bone graft with a theoretical drug release profile of a non-limiting bone implant of the present disclosure over time. As shown in FIG. 6A, a certain level of drug is toxic. Below this threshold, there is a dosage level at which the drug will kill bacteria. However, below this dosage level is a level where the presence of drug will actually encourage the development of bacteria resistant to the drug. As shown in FIG. 6A, the current bone graft (shown in a red dotted line) releases an initial bolus of drug shortly after implant (e.g., within two weeks of implant), but then drug release quickly tapers off. However, the tapering off period of the current bone implant may occur before all the bacteria at the site are killed, and thus bacteria resistant to the drug may develop. However, a non-limiting implant as disclosed herein (shown as a solid blue line in FIG. 6A) maintains a sustained release of drug for much longer. The implant of the present disclosure has an initial bolus of drug release, but then maintains a sustained drug-release level high enough to kill bacteria for a prolonged amount of time (see solid blue line, FIG. 6A). Finally, as the implant disclosed herein fully dissolves and is replaced by in-growth of the patient's own bone, a second bolus of drug release occurs (see peak of the solid blue line at the 6-8 week time point in FIG. 6A). This second bolus may kill off any drug-resistant bacteria that may have developed.

FIG. 6B shows the actual (and not theoretical) drug release profile from a non-limiting implant of the disclosure (solid blue line) as compared to the rate of the reformation of the patient's bone (red line). As FIG. 6B shows, there is an initial bolus of drug released before 24 hours post-implantation, and then a second bolus released at about five hour post-implantation. Still later, a much smaller bolus of drug release occurs at about 6.5 weeks, with the drug release from the implant sustained past 12 weeks post-implantation. Note that drug release curve in FIG. 6B is from a generation 2 implant, but the histological image superimposed onto FIG. 6B and shown enlargened in FIG. 6C is from a generation 3 implant, but this histological image is thought to be typical of any generation (i.e., generation 1, 2, or 3) of ElutiBone fabrication. The histological image in FIG. 6B and FIG. 6C depicts a generation 3 implant of the disclosure (labeled as ElutiBone graft in FIGS. 6B and 6C) adjacent to the patient's bone in situ.

Currently, while synthetic bone graft materials have a lengthy clinical pedigree, no FDA approved allograft bone therapies incorporate an integrated antibiotic release scheme as combination medical devices. In accordance with various embodiments of the present invention, the bone graft is acting in its primary mode of action as a medical device (bone graft filler) and the drug-releasing modality is a secondary mode of action. In addition to polymer barrier coating characteristics, other factors can be exploited in this modular combination device approach. Graft surface area (micron-scale morselized bone can be milled to have a higher surface area for drug release than cancellous crouton fragments), diverse differential implant packing (i.e., mixing of large allograft cortical croutons with morselized allograft cancellous granules either as separate coated formulations or within a single coated preparation) and antibiotic solid microencapsulation (e.g., in common, clinically routine starch or solid-dosage form encapsulating matrices) prior to drug dose loading all provide a range of customizable drug loading and release options appropriate for tailoring and customizing bone defect combination devices for better mitigating infectious risks in orthopedic and connective tissue surgical implant and repair sites. While directly soaking allograft bone filler materials in antibiotic has been studied extensively (see, e.g., Witso et al., Acta Orthop, 76 (2005): 481-486; Witso et al., Acta Orthop Scand, 70 (1999): 298-304; Witso et al., Acta Orthop Scand, 71 (2000): 80-84; Darley and MacGowan, J Antimicrob Chemother, 53 (2004): 928-935; Rhyu et al., Int Orthop, 27 (2003): 53-55; Winkler et al., J Antimicrob Chemother, 46 (2000): 423-428; Witso et al., Acta Orthop Scand, 75 (2004): 339-346; Lindsey et al., Clin Orthop Relat Res. 291: 303-312 (1993), the idea of endowing this matrix with a true local controlled release strategy has not.

In some embodiments, the non-limiting allograft and synthetic bone matrix-antibiotic-polymer combination devices (i.e., the bone implants) described herein (and shown schematically in the Figures) permit precise, uniform tobramycin drug loading (via the polymer overcoat) to retain the drug release depot at the surgical site controlled by polymer (PCL (co)polymer±PEG mixtures) coating swelling, porosity and degradation by hydrolysis. The bone implants described herein exhibit long-term antibiotic release at the wound or implantation site and maintenance of therapeutic antimicrobial drug concentrations at the implantation site beyond 6 weeks, beyond 8 weeks, or even beyond 10 weeks post-implantation. In some embodiments, the release (i.e., diffusion) of the drug at a therapeutic levels (e.g., locally at the implantation site) is maintained for at least eight weeks. In some embodiments, the release of the drug at a therapeutic level (e.g., at the implantation site) is maintained for a time longer than the amount of time a pathogen can remain in either a metabolically active or a senescent state (e.g., in a biofilm).

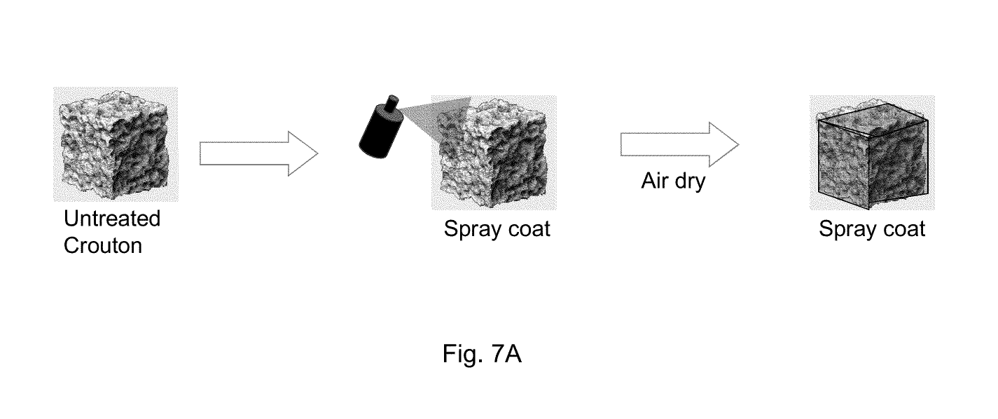

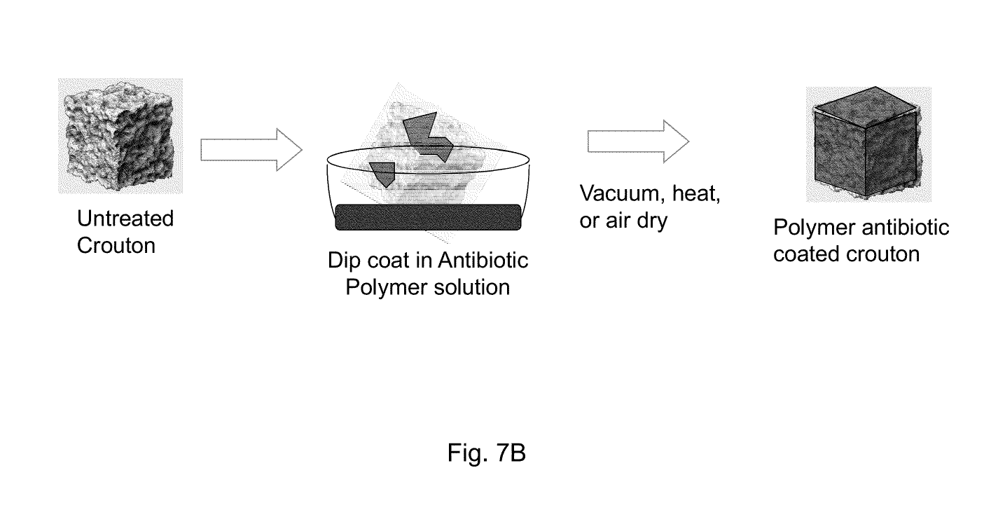

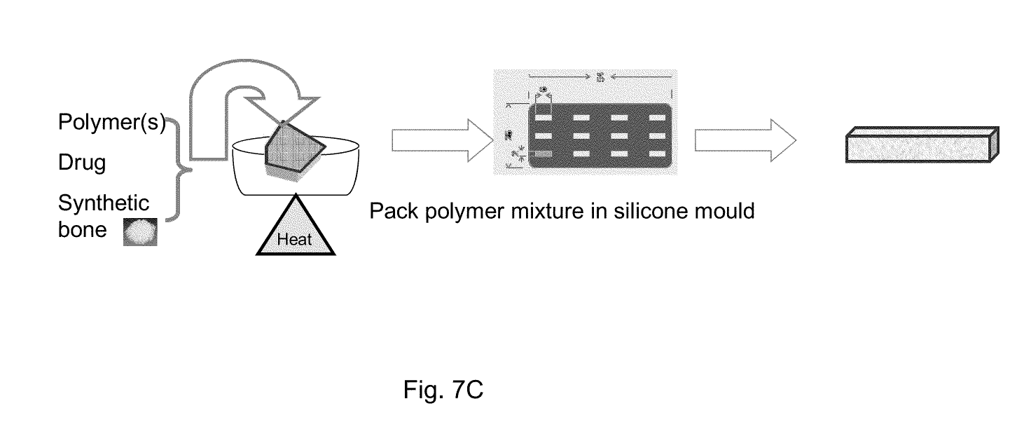

In some embodiments, the versatility of at least some of the bone implants of the invention (and fabrication methods thereof) is depicted schematically in FIGS. 7A-7C. In one non-limiting embodiment shown in FIG. 7A to generate a non-limiting bone implant of the invention (which may be referred to herein as the generation 1 fabrication), the untreated crouton of bone material (e.g., synthetic or allograft bone material) is spray coated with an antibiotic-containing polymer-containing solution and then dried (e.g., air-dried, vacuum-dried, or heat-dried). In another embodiment shown in FIG. 7B to generate a non-limiting bone implant of the invention (which may be referred to herein as the generation 2 fabrication), starting with an untreated crouton of bone material (e.g., synthetic or allograft bone material), the crouton is first dipped into an antibiotic-containing polymer-containing solution and then dried (e.g., air-dried, heated or vacuum dried) to create a polymer antibiotic-coated crouton. The starting crouton of bone material may also be soaked in an antibiotic-containing solution first (e.g., soaked and then dried) prior to dipping the antibiotic-soaked crouton into an antibiotic-containing polymer-containing solution and then drying to create a polymer antibiotic-coated, antibody-soaked crouton.

In yet another embodiment (FIG. 7C), the polymer, drug, and synthetic bone material are mixed together, heated, and then packed into a silicon mold with wells of precise dimensions. The results of this fabrication method (referred to as the generation 3 fabrication method) are multiple coated implants of precise dimensions. Of course, practitioners will understand that antibiotic release kinetics will depend upon the polymer formulation and fabrication method.

In some embodiments, an implant (e.g., generated using the generation 3 fabrication method) can be made of a polymer component, a bone component, and a drug component. In some embodiments, for example, the bone component may be ground or morselized and/or may be natural bone or synthetic bone (e.g., ProOsteon). In some embodiments, for example, the drug may be an antibiotic such as tobramycin. In some embodiments, for example, the polymer component may be PCL, or may be a PCL and a PEG combination, or may be a PCL, a PEG, and a poly(lactide-co-glycolide) combination. In some embodiments, the polymer component may also include a poragen such as calcium chloride. Calcium chloride is a biocompatible water-soluble salt, which is being looked at as pore former in the modified formulation. This water-soluble salt is expected to dissolve in less than 24 hours to create initial porosity to allow ingress of fluid and cells. The poly(lactide-co-glycolide) will degrade faster than the PCL and will release the initial tobromycin load. It is expect the slower degrading PCL will then deliver the later drug load. Poly(lactide-co-glycolide) is a biocompatible degradable polymer used commonly in sutures, fracture fixation deices and microsphere in drug delivery, It is more hydrophilic than PCL and as such degrades faster than PCL.

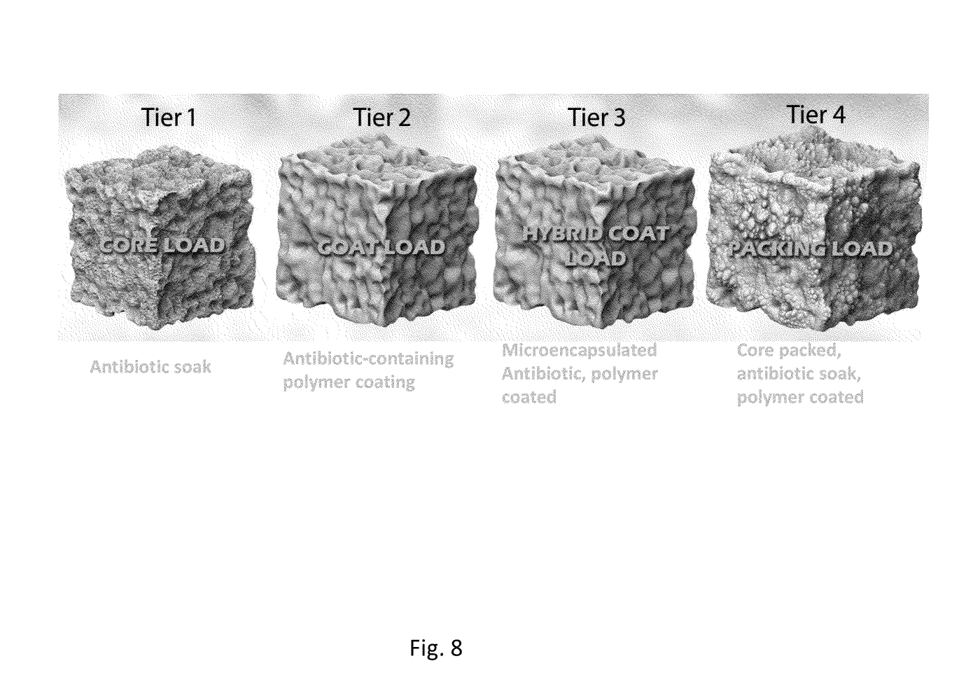

FIG. 8 shows the formation of additional non-limiting bone implants. In FIG. 8, tier 1, a bone material crouton is coated with an antibiotic-containing polymer coating to generate a bone implant. In tier 2 of FIG. 8, a bone material crouton is coated with an antibiotic-containing polymer (e.g., PCL) solution. In tier 3 of FIG. 8, the non-limiting implant is generated by mixing a microencapsulated antibiotic with a polymer to create a microencapsulated antibiotic polymer solution which is then used to coat a bone material crouton. In tier 4 of FIG. 8, a bone material crouton (e.g., packed with a product such as demineralized bone matrix) is soaked in an antibiotic-containing solution first, and then the antibiotic-soaked bone material crouton coated with a polymer (e.g., a polymer coat that may or may not have been mixed with an antibiotic prior to use as a coating).

Note that in FIG. 8, each of the four tiers may represent four different non-limiting bone implants of the invention. Additionally, the four tiers may be combined with one another to create additional bone implants. For example, the tier 3 product (i.e., the hybrid coat load) made by coating a bone material crouton with a microencapsulated antibiotic-containing polymer solution may be first soaked in an antibiotic-containing solution (as in the tier 1 product) prior to coating. In other words, an additional bone implant of the invention includes a tier 1 product coated with an microencapsulated antibiotic-containing polymer coating to result in an antibiotic-containing bone material coated with an antibiotic-containing polymer coating. Additionally note that in FIG. 8, polycaprolactone (PCL), tobramycin, and synthetic ceramic bone graft void filler is used; however, other materials can be used (e.g., a different antibiotic such as gentamycin or cadaver allograft bone material).

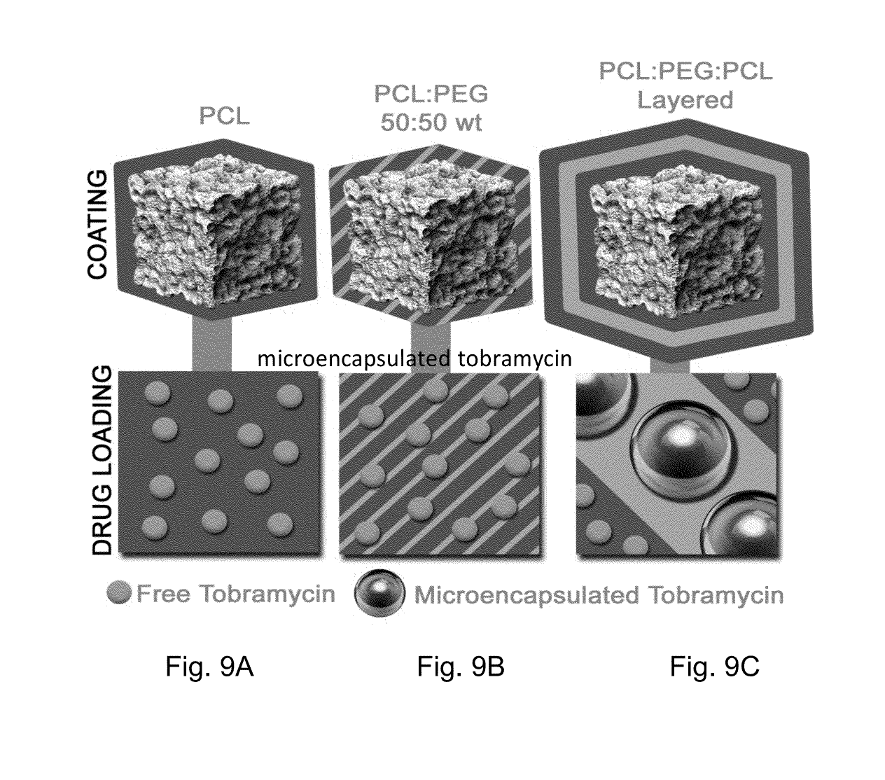

FIGS. 9A-9C show details of some of the non-limiting bone implants of the invention. In FIGS. 9A-9C, the generation 2 fabrication (generated, for example, using the method depicted in FIG. 7B) was modified. For FIGS. 9A-9C, free tobramycin is shown in small light green circles while micro-encapsulated tobramycin is shown in larger darker green circles. Note that in the non-limiting bone implants depicted in FIGS. 9A-9C (and/or described in any of the figures or the text of this disclosure), tobramycin is simply shown as an example drug—other drugs can be used including, without limitation, gentamycin, ciprofloxacin, rifampicin, vancomycin, oxacillin, verdamicin, astromicin, doxycycline, tetracycline, streptomycin, neomycin, kanamycin, spectinomycin, linezolid, clindamycin, and erythromycin. Additional anti-bacterial drugs are well known (see, e.g., Kucers' The Use of Antibiotics, 6^{th} Ed., Ed. M. Lindsay Grayson et al., American Society for Microbiology, Published by Hodder Arnold, 2010.

In FIG. 9A, PCL (which is shown in dark blue) is mixed with free tobramycin and this mixture is used to coat the synthetic or allograft bone to result in the standard generation 2 fabrication. FIG. 9B shows a bone implant generated using a modification of the generation 2 fabrication process. In FIG. 9B, free tobramycin is mixed in with both PCL (dark blue in FIG. 7B) and PEG (light blue in FIG. 7B), and the resulting mixture is used to coat the synthetic or allograft bone

FIG. 9C shows a further modification of the standard generation 2 fabrication. First, free tobramycin is mixed into PCL to create a free tobramycin:PCL solution. Another solution (namely a PEG:microencapsulated tobramycin solution) is made by mixing a PEG solution with microencapsulated tobramycin. To generate the bone implant shown in FIG. 9C, the synthetic bone (or allograft bone) crouton is first dipped into the PCL: free tobramycin solution, and then dried in vacuum. The dried crouton is next dipped into the PEG: microencapsulated tobramycin solution, and then dried in a vacuum. The dried two-layer coated crouton is then dipped again in the PCL: free tobramycin solution and dried in the vacuum. The resulting bone implant (shown in FIG. 9C) has three layers of coating, namely an innermost (i.e., directly on the bone crouton) and outermost layer of PCL:free tobramycin with a middle layer of PEG: microencapsulated tobramycin. The weight of the dried crouton was obtained after each stage.

As shown in FIGS. 9A-9C, depending upon the coating used, the bone implants are coated with drug (in this case, tobramycin) that is either free in the PCL coating (FIG. 9A), interspersed with polyethylene glycol (PEG) and PCL in the coating (FIG. 9B), or is microencapsulated in the PEG layer and free in the PCL layers, when the coating is PCL:PEG:PCL layered (FIG. 9C).

In accordance with the present disclosure, the implants described herein (e.g., those depicted in FIGS. 7, 8, and 9) can be used, for example, to fill a bone defect, to provide scaffolding support (e.g., to the animal's cells such as osteoblasts), to provide sustained, local antibiotic delivery for over six to eight weeks post-implant (or for over eight to ten weeks post-implant, or for over ten weeks post-implant). The implant is osteoconductive, versatile, and is a major clinical improvement over current technologies.

Accordingly, in a first aspect, the invention provides an implant comprising, consisting, or consisting essentially of a uniform mixture of degradable polymer, bone, and a drug. In some embodiments, the drug comprises an antibiotic. In some embodiments, the implant is configured so that upon implantation of the implant into a host at an implantation site, the drug diffuses from the implant at a therapeutic level. In some embodiments, the host is a vertebrate animal. In some embodiments, diffusion of the drug from the implant at a therapeutic level is maintained for at least eight weeks post-implantation. In some embodiments, diffusion of the drug from the implant at a therapeutic level is maintained for at least ten weeks post-implantation. In some embodiments, diffusion of the drug from the implant at a therapeutic level is maintained for at least twelve weeks post-implantation. In some embodiments, the therapeutic level is maintained at an implantation site of the implant.

In some embodiments, the implant is a solid. In some embodiments, the implant is molded. In some embodiments, the implant is carvable, so that it may be shaped prior to implantation. In some embodiments, the implant is shaped for use with an implantable prosthesis. In some embodiments, the implant is shaped for use with an implantable prosthesis. In some embodiments, the prosthesis is a fixation tooling, a plate, a screw, a rod, a pin, a nail, or a total arthroplasty of various forms used clinically in orthopedic surgery.

In some embodiments, the implant is a liquid. In some embodiments, the implant is a paste. In some embodiments, the implant is a putty. In some embodiments, the implant is a coating on an implantable prosthesis. In some embodiments, the prosthesis is of a material selected from the group consisting of a metal (including, for example, a metal oxide), a ceramic, a porcelain, an alloy, and a combination of two or more of the foregoing.

In some embodiments, the implant is configured so that upon implantation of the implant, the drug diffuses from the implant in a manner to provide a first bolus after a first period of time following implantation and a second bolus after a second period of time following implantation. In some embodiments, the first period is about one week and the second period is about five weeks. In some embodiments, the first period is about one day and the second period is between about three weeks and about six weeks.

In some embodiments of the implant, the bone is present in the uniform mixture in a first quantity by weight and the degradable polymer is present in the uniform mixture in a second quantity by weight, wherein the first quantity is greater than the second quantity. In some embodiments, the first quantity is at least 1.125 times larger than the second quantity, or is at least 1.25 times larger than the second quantity, or is at least 1.5 times larger than the second quantity, or is at least two times larger than the second quantity, or is at least 2.25 times larger than the second quantity, or is at least 2.5 times larger than the second quantity.

In another aspect, the invention provides a method of making a solid implant, the method comprising: making a uniform mixture including degradable polymer, bone, and a drug; forming the mixture into a desired shape; and curing the shaped mixture to form a solid implant. In some embodiments, the curing step includes subjecting the shaped mixture to heat.

In various embodiments, the bone is present in the uniform mixture in a first quantity by weight and the degradable polymer is present in the uniform mixture in a second quantity by weight, wherein the first quantity is greater than the second quantity. In some embodiments, the first quantity is at least 1.125 times larger than the second quantity, or is at least 1.25 times larger than the second quantity, or is at least 1.5 times larger than the second quantity, or is at least two times larger than the second quantity, or is at least 2.25 times larger than the second quantity, or is at least 2.5 times larger than the second quantity.

In another aspect, the invention provides an implantable bone void filler comprising a polycaprolactone (PCL) polymer, an antibiotic, and a bone fragment. In some embodiments, the antibiotic is selected from the group consisting of tobramycin, ciprofloxacin, and vancomycin.

In some embodiments, the implant is in contact with (e.g., in combination with or coated onto) a prosthetic. In some embodiments, the prosthetic is implanted. By “prosthetic” is meant a wholly artificial structure that is or can be implanted into a vertebrate host animal to aid in functional restoration of a tissue, including bone. Prosthetics include, without limitation, metal prosthetics (e.g., titanium, steel, gold, platinum, etc.), ceramic, and porcelain in the form of multiple tools and stabilizing, or structural aids, including plates, screws, rods, cannulae, fusion cages, nails, pins, meshes, cups, sutures, and joint arthroplasty devices. The prosthetic need not be solid. For example, a prosthetic may be porous. A prosthesis may also be flexible, or may be both porous and flexible. In some embodiments where the prosthesis is porous and the implant is liquid, the liquid implant may coat the surfaces or walls of the pores of the prosthesis. Such coating may be done prior to implantation, or during implantation.

### Example 1

In this example, an implant was fabricated and tested for its ability to diffuse drug for a prolonged amount of time in vitro.

For these studies, the following methods were used.

Fabrication of polymer-coated allograft fragments. To do this, cancellous allograft bone fragments (Miami Tissue Bank) or ProOsteon 500R (BioMet, Warsaw, Ind., USA) were weighed and like-size and mass fragments were selected for each cohort (n=3). Alternatively, micron-size allograft bone particulate matter (Miami Tissue Bank, Miami, Fla., USA) was partitioned into 100 mg aliquots for polymer-drug coating. PCL (10 kD, Sigma Aldrich, St. Louis, Mo., USA) (60 mg/ml) was dissolved in acetone (Thermo Fisher Scientific, Waltham, Mass., USA) at 45° C. Tobramycin (MP Biomedicals, Solon, Ohio, USA) was suspended as “free” (i.e., unencapsulated) drug in PCL acetone solutions at 10% weight/volume. Alternatively, certain PCL coating formulations included tobramycin commercially microencapsulated in vegetable triglycerides (70 w/w % tobramycin, lot#TM150-70-30, Maxx Performance Inc., Chester, N.Y., USA). Formulations and cohorts are detailed in Table 1.

TABLE 1
Allograft bone drug-loaded cohorts used

| | PCL | PEG | Tobramycin (w/v) | | Application | Graft | Graft |
|---|---|---|---|---|---|---|---|
| Cohort | (10 kD) | (w/v) | unencapsulated | encapsulated | Technique | Substrate | form |
| 1 | 60 mg/ml | 0 | 10% | 0 | Dip-coat | allograft | Fragment |
| 2 | 60 mg/ml | 0 | 10% | 0 | Dip-coat | ProOsteon | Fragment |
| | | | | | | 500R ® | |
| 3 | 60 mg/ml | 0 | 10% | 0 | Solvent | allograft | Particulate |
| | | | | | evaporation | | |
| 4 | 60 mg/ml | 45% | 10% | 0 | Solvent | allograft | Particulate |
| | | | | | evaporation | | |
| 5 | 60 mg/ml | 45% | 0 | 10% | Solvent | allograft | Particulate |
| | | | | | evaporation | | |
| 6 | 60 mg/ml | 45% | 5% | 5% | Layer-by- | allograft | Particulate |
| | | | | | Layer | | |

Each cohort was made with 10 kD PCL dissolved in acetone at 45 C. If PEG was included then it was dissolved in water first and then the PEG water solution was added to the PCL acetone solution. All tobramycin (regardless of encapsulation state) was added to the polymer solution(s) as a dry powder to create the formulation used to either dip coat allograft or ProOsteon fragments or to solvent cast the particulate as described in the next paragraph and schematically depicted in FIGS. 7B and 9A-9C. The solvent used with the fabrications in all of these cohorts was acetone. These fabrications were generated with the PEG dissolved in water and the PCL being dissolved in acetone. [HOW IS COHORT 6 MADE?] An unloaded polymer bone control was included in all analyses (data not shown). Dip-coated cohorts were prepared by placing allograft bone into the PCL/free tobramycin solution at room temperature.

This fabrication method is schematically depicted in FIG. 7B and in FIGS. 9A-9C. The bone fragments were removed after soaking in polymer solution for 30-60 seconds. After vacuum drying (5-10 minutes at ambient temperature), each fragment was weighed again to determine amounts of drug and polymer applied. Allograft particulate cohorts of identical mass were coated in individual aluminum trays with 2 ml of polymer/drug solution, mixed twice and then subsequently the solvent was allowed to flash off, leaving coated particulate. The particulate-containing polymer film was ground using a weighing spatula prior to placing it in a 50 ml conical vial for release into PBS. To alter drug release kinetics, 45% PEG (20 kD, Sigma Aldrich, St. Louis, Mo., USA) and/or microencapsulated tobramycin were either mixed directly with the PCL solution or coated in alternating layers with it. Allograft particulate was coated in individual aluminum trays with a total of 2 ml of polymer/drug solution (500 ul PCL with free tobramycin, 1 ml PEG with microencapsulated tobramycin, 500 ul PCL with free tobramycin) in a layer-by-layer (LBL) fashion with alternating layers of PCL and PEG. To create a polymer/drug layer the particulate was mixed twice in each polymer/drug solution and the solvent was allowed to flash off, leaving coated particulate. The particulate-containing polymer film was ground using a weighing spatula and the next layer was applied according to the same protocol.

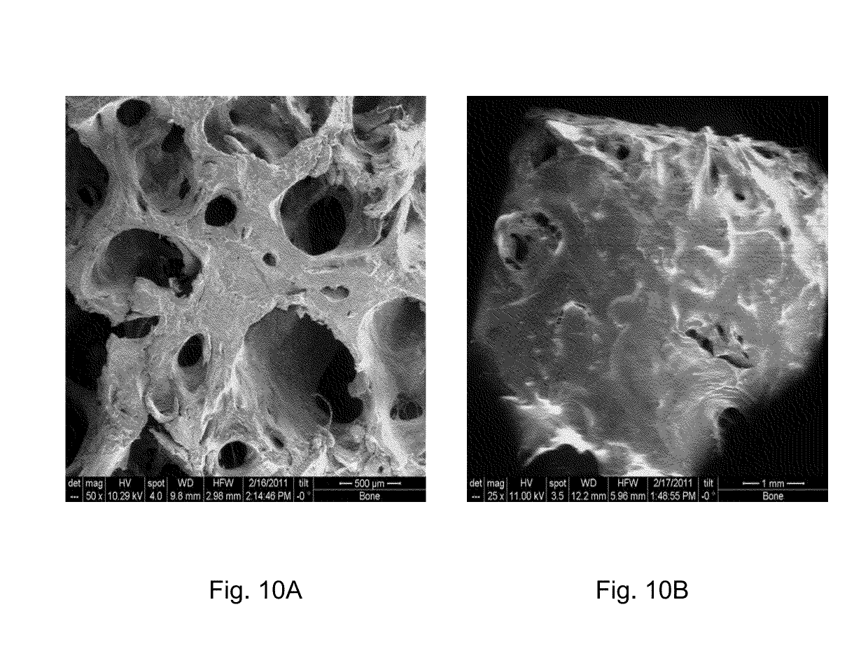

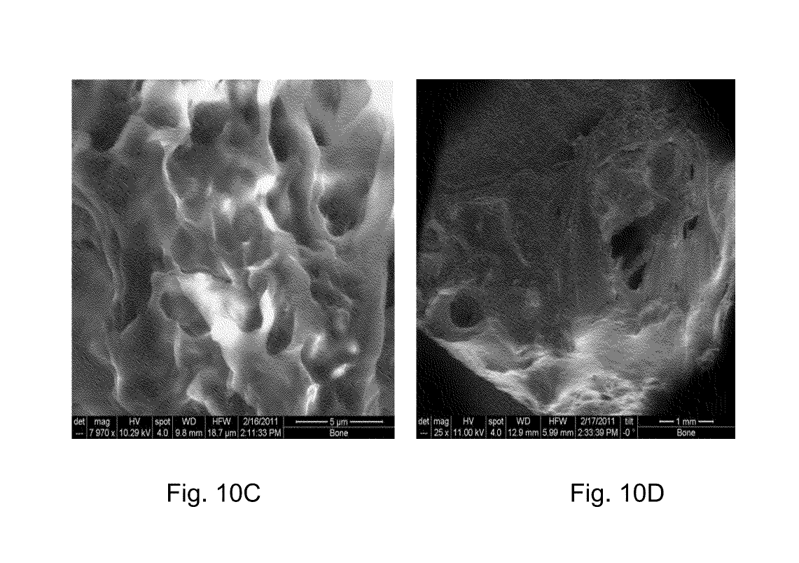

Note that for cohort 6, allograft particulate was coated in individual aluminum trays with a total of 2 ml of polymer/drug solution (500 ul PCL with free tobramycin, 1 ml PEG with microencapsulated tobramycin, 500 ul PCL with free tobramycin) in a layer-by-layer (LBL) fashion with alternating layers of PCL and PEG. To create a polymer/drug layer, bone graft particulate was mixed twice in each polymer/drug solution and the solvent was allowed to flash off, leaving coated particulate. The dried particulate-containing polymer film was ground to granules again using a weighing spatula and the next layer was applied according to the same protocol. FIGS. 10A-10D are scanning electron microscopy (SEM) images of four representative (but non-limiting) fabricated bone implants incorporating different antibiotics (i.e., FIG. 10A is uncoated; FIG. 10B with tobramycin sulfate, FIG. 10C with ciprofloxin HCl, and FIG. 10D with vancomycin HCl. As can be seen from the different images, an open or a closed porous implant can be achieved by changing the drying techniques (e.g., vacuum, air-drying, or heat-drying). Vacuum drying gives a slightly more open pore structure although many of the pores are still occluded. Note that air drying and heat drying are virtually indistinguishable.

Drug Release Measurements.

To measure drug release from the fabricated implants, each coated allograft bone sample was placed into 3 ml of phosphate buffered saline pH 7.4 (PBS, Fisher Scientific, Waltham, Mass., USA). The complete volume (called the release volume because it contains the released drug) was drawn off and replaced at 30 minutes, 1 hour, 2 hours, 4 hours, 8 hours, 24 hours, 72 hours, and each week for up to 6 weeks to simulate sink conditions. Kinetics of release from each formulation were assessed via a 96-well fluorescent assay previously reported (Sevy et al., Biomed Sci Instrum, 46 (2010) 136-141). Briefly, 75 ul of each release sample was added to 75 ul of isopropanol in wells within black-masked 96-well plate (Fisher Scientific, Pittsburgh, Pa., USA). OPA working solution (150 ul) (50 ul of o-phthaldehyde (OPA, Sigma Aldrich, St. Louis, Mo., USA) stock solution in 1 ml of 0.5M potassium borate buffer pH 10.5) was added to each well and incubated for 30 minutes prior to assessing the fluorescence of the tobramycin/OPA derivative (Biotek spectrophotometer, ex=360 nm, em=460 nm) using Gen5 1.09 software (BioTek, Winooski, Vt., USA). Each cohort contained a certain number of reference samples (n=3, 6, or 9) from which tobramycin was not released over time but instead the entire coating was dissolved in 1 ml of chloroform (Thermo Fisher Scientific, Waltham, Mass., USA) for approximately 5 minutes and 1 ml of water was used to phase extract tobramycin from the polymer solution by vortexing for 30 seconds and then centrifuging at 15,000 rpm for 2 minutes and 30 seconds. These samples were considered 100% release samples and all amounts of tobramycin released over time from coated grafts were normalized to their cohort-matched 100% release value as well as to the unloaded polymer bone control, and reported as a percent to facilitate direct comparison of release from different polymer formulations. Tobramycin from fragments coated with a PCL-water non-solvent system was phase extracted after 8 weeks of release into PBS using chloroform and water to verify the mass balance of the system (data not shown).

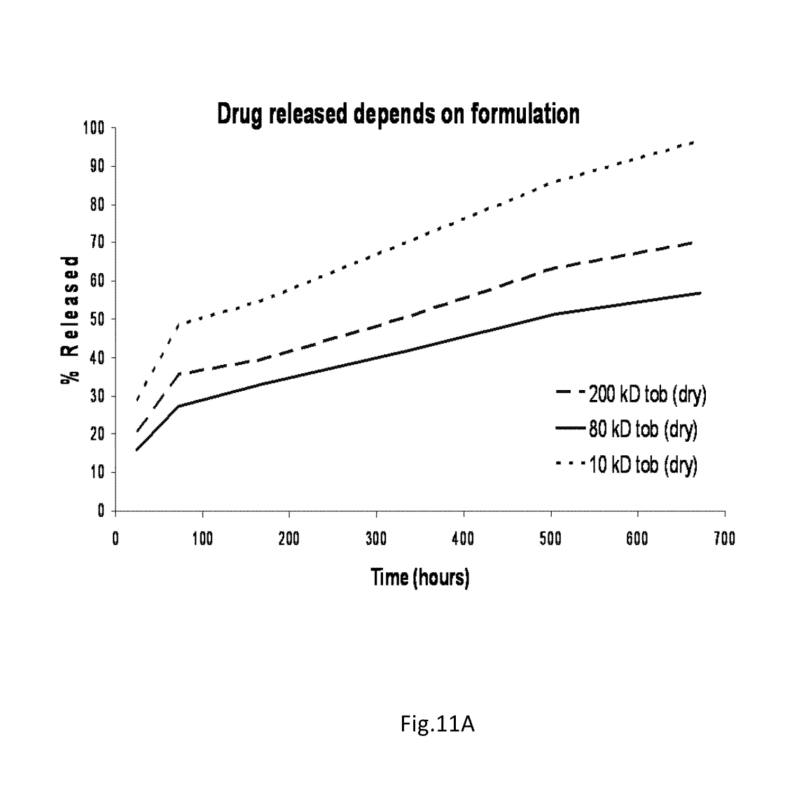

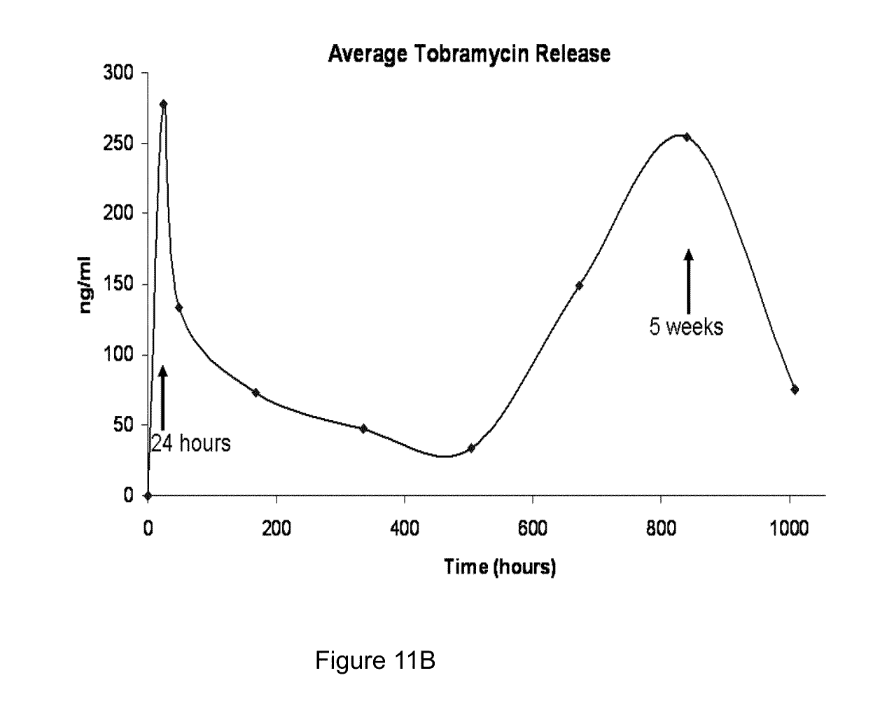

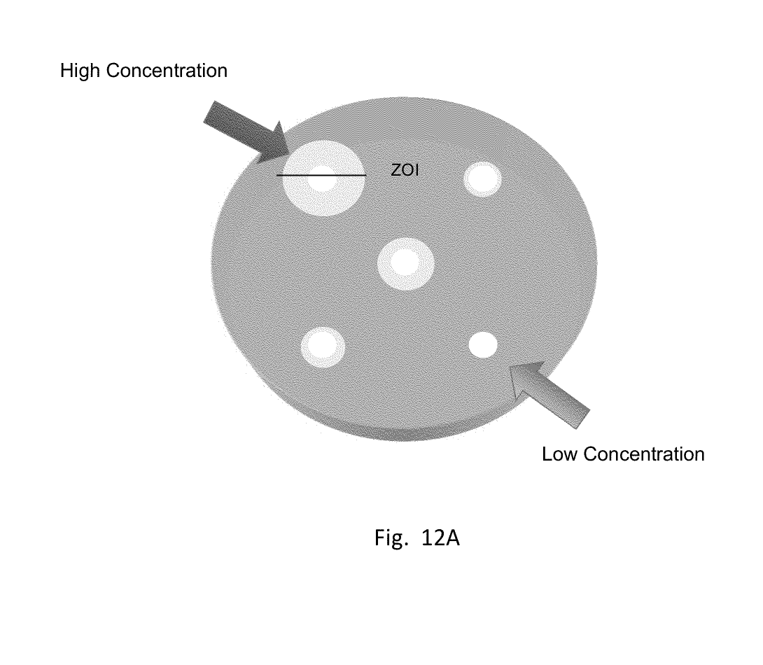

As shown in FIGS. 11A and 11B, the rate of release of the antibiotic is controllable by tunable degradation. In FIG. 11A, three different forms of tobramycin: polymer coating (i.e., 200 kD PCL, 80 kD PCL, and 10 kD PCL at 60 mg/ml each in acetone or, for formulation (i.e., cohorts 4-6) that include PEG, a mixture of acetone and water where the PEG is dissolved in water and the PCL is dissolved in acetone) with 10% weight/weight tobramycin in the coating formulation were used in to fabricate the implants, and the amount of tobramycin released was measured post-implantation using the methods described here. As can be seen from FIG. 11A, the implanted coated with the 10 kD polymer: tobramycin (weighed when the polymer is dry; dotted line) released its drug faster than the implant coated with 80 kD polymer: tobramycin (dry weight of the polymer; solid line) or the implant coated 250 kD polymer: tobramycin (dry weight of polymer; dashed line) released their drug.

FIG. 11B shows the tobramycin release from allograft croutons dip-coated with a 10% weight/volume powdered tobramycin containing coating solution with the 80 kD PCL polymer at 60 mg/ml in acetone with the inclusion of a 4% volume/volume water non solvent. As can be seen in this non-limiting example in FIG. 11B, following the initial peak of tobramycin release at 24 hours, a second peak at approximately 5 weeks occurs. Thus, the rate of antibiotic release is controlled by polymer/antibiotic drug formation, and can also be controlled by the fabrication method.

In various embodiments of the invention, the ability to modulate drug release kinetics is useful for combating bacterial infection. Indeed, drug release can be tailored to match the rate of bone growth and remodeling. As shown in FIG. 6B, the release of tobramycin data from FIG. 11B is super-imposed onto a graph showing the rate of bone formation, where bone is first removed by osteoclasts, and then reformed by osteoblasts. Note that the graph shown in FIG. 6B shows drug release data collected past the six weeks post-implantation time point shown in FIG. 11B—FIG. 6B shows drug release data after twelve weeks post-implantation. Thus, the non-limiting tobramycin-releasing implant shown in FIGS. 6B, 11A, and 11B is able release tobramycin to match the rate of bone growth and remodeling, thus supporting bone growth and preventing infection. This will allow the graft to be remodeled into host bone over time.

Data Analysis:

The amount of tobramycin released in each sample was calculated based on the linear regression of the fluorescent units (FU) for each standard. Percent drug release was calculated by dividing the amount of tobramycin released by the amount of tobramycin detected in the dissolved coating (100% release) multiplied by 100. All formulations were tested in triplicate (biological and technical replicates) and Excel was used to calculate the propagating standard deviation. Pairwise one-way ANOVAs were used to identify significant differences (p<0.05 for significance). Particular comparisons to be tested were selected in advance and were reported individually rather than as a group and therefore a multiple comparison correction was not necessary (see Dunnett and Goldsmith, “When and how to do multiple comparisons”, in: C. R. Buncher, J.-Y. Tsay (Eds.) Statistics in the Pharmaceutical Industry, Chapman and Hall/CRC New York, 2006, pp. 421-452).

High Performance Liquid Chromatography (HPLC):

Standard concentrations of tobramycin were resuspended in acetonitrile-water (52:48). All samples were analyzed in triplicate using high pressure liquid chromatography (HPLC), with a pre-column OPA derivatization (see Sevy et al., Biomed Sci. Instrum., 46 (2010) 136-141). Data was collected from both a fluorescence detector (ex=350 nm, em=450 nm) as well as UV-V is detector (340 nm). Samples were analyzed using a Hypersil GOLD HPLC column (100×4.6 mm, Thermo Fisher Scientific, Waltham, Mass., USA) and ChromQuest 5.0 (Thermo Fisher Scientific, Waltham, Mass., USA) software on a Finnigan Surveyor (Thermo Fisher Scientific, Waltham, Mass., USA) system. Each sample (10 ul) was injected using a 2 ml/min flow rate. The mobile phase was mixed 0.02M phosphate (pH 6.5):acetonitrile (52:48). The area under the tobramycin peak was plotted against standard concentration and data were fit by linear regression as a standard curve, used to calculate the concentration of unknown drug release samples.

Microbiology:

Release samples (500 ul per experiment) for all microbiology studies were concentrated in a vacuum centrifuge (Labconoco Centrivap, Kansas City, Mo., USA) overnight at ambient temperature and prepared in low-bind, non-tissue culture-treated 96-well microtiter plates according to their subsequent experimental use (i.e., MIC: round bottom, ZOI: flat bottom). All samples were stored dry at 4° C. until use. Antimicrobial activity after concentration as well as storage was confirmed with control conditions.

Bacteriostatic Assay:

LB broth (100 ul, Becton Dickinson, Franklin Lakes, N.J., USA) was added to each well of the round bottom 96-well plate to reconstitute the dried drug release samples. Each well was inoculated with 10⁵ CFU in 200 ul of a liquid culture of E. coli (ATCC 25922, American Type Culture Collection, Manassas, Va., USA). Liquid bacterial cultures were prepared using a sterile swab to select 1-3 isolated colonies from a blood agar plate (Remel, Lenexa, Kans., USA). Inoculated plates were incubated overnight at 37° C. Released drug activity, assessed by bacterial growth inhibition, was visually determined by comparing known standard tobramycin concentrations. Growth inhibition was positive if the visual turbidity of bacterial growth media differed from the positive control by 80%. Negative growth was designated when the well was free of a visible bacterial pellet.

Zone of Inhibition (ZOI):

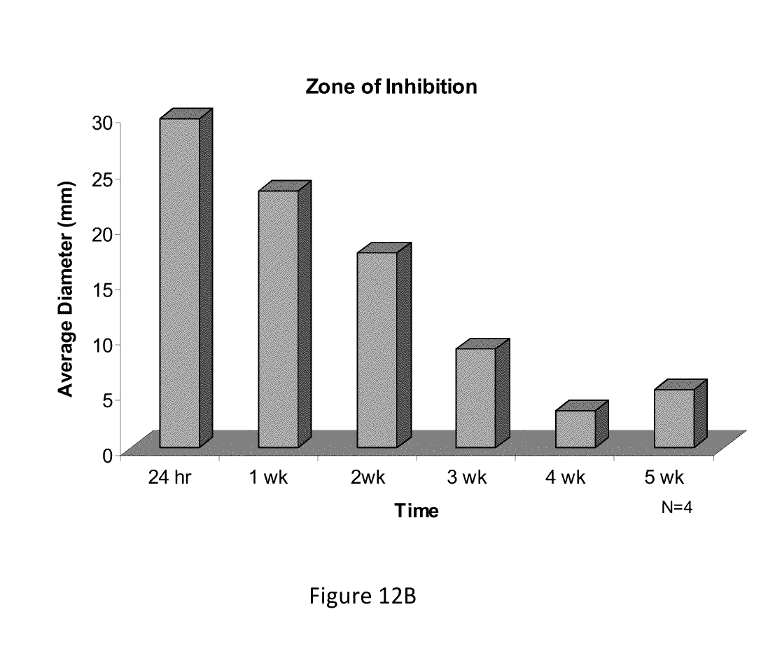

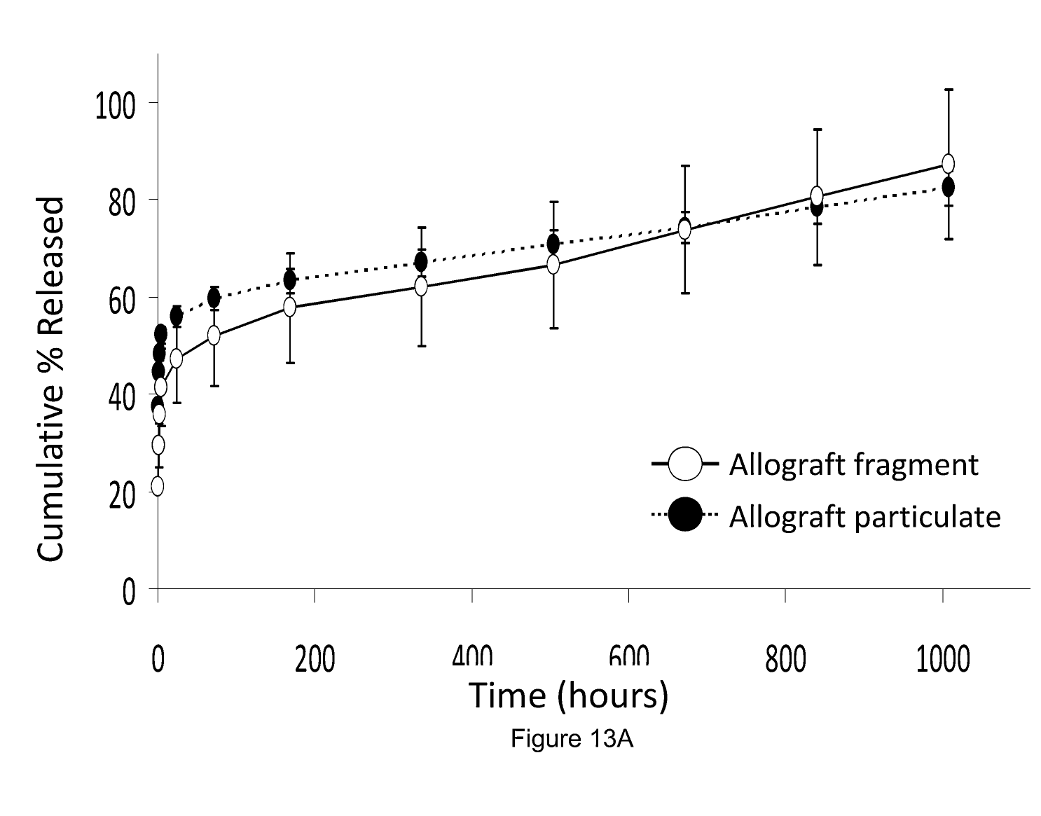

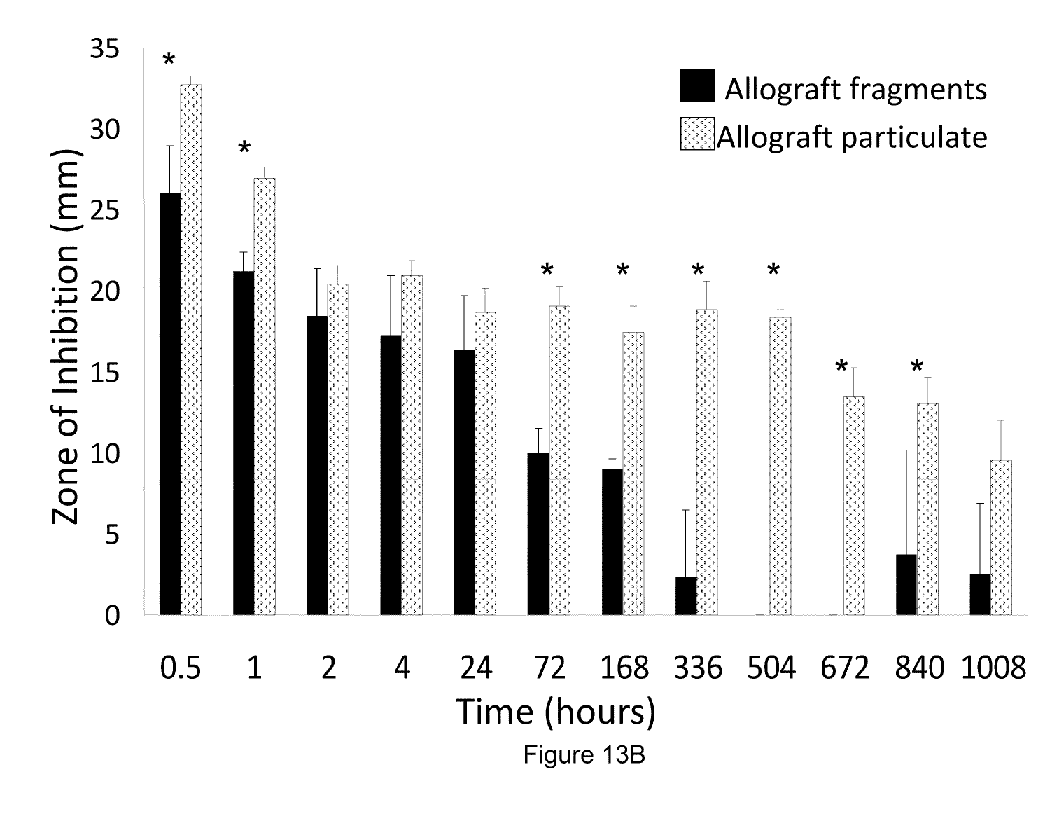

For ZOI experiments, release samples were dried onto 6 mm Whatman 1 filter paper disks. Muller Hinton agar plates (Fisher Scientific, Waltham, Mass., USA) were prepared by streaking E. coli (ATCC 25922 from American Type Culture Collection, Manassas, Va.) to create a confluent lawn of bacterial growth (turbidity adjusted to a 0.5 McFarland standard using a nephelometer (Phoenix Spec, BD Diagnostic Systems, Franklin Lakes, N.J., USA)). Disks containing the dried-down drug from release samples were then placed with a minimum distance of 24 mm between each disk and the side of the plate. Plates were incubated overnight at 37° C. Calipers were used to measure the diameter of the zone of inhibition around each disk. FIGS. 12A and 12B depict the results of a typical ZOI assay with a non-limiting bone implant described herein. As shown in FIG. 12A, the diameter of the cleared area in the bacterial lawn is proportional to the amount of drug on the dried-down disk (e.g., the diameter shown in the black line at the high concentration arrow is approximately three times the diameter of the disk itself). FIG. 12B shows a typical bar graph resulting from such a ZOI assay for a non-limiting bone implant. Over 2000 zone of inhibition assays were done for different formulations against lawns of E. coli bacteria and S. aureus bacteria. FIGS. 13B, 14B, 15B, 16B, and 17B shown representative results from some non-limiting formulations for bone implants of the invention.

Sample Fabrication and Drug Release Assay. Tobramycin is a clinical drug of choice used to treat orthopedic infections; however, due to associated nephro- and ototoxicity (Begg and Barclay, B. J. Clin. Pharmacol. 39: 597-703, 1995), maintaining adequate drug concentration to combat opportunistic microbes in bone using traditional delivery mechanisms (intravenous) may be unachievable. Therefore, a local, polymer-controlled delivery of tobramycin directly to the bone using a bone graft delivery vehicle was investigated. Samples were fabricated according to Table 1 with all allograft fragments (approx. 6 mm×5 mm×4.5 mm) and ProOsteon 500R® fragments (approxl Omm×8 mm×7 mm) being dip-coated to add approximately 22 mg of drug-releasing coating to the fragment's initial weight. Importantly, cohorts of bone graft fragments were weight matched to limit their variability. Alternatively, micron-size allograft particulate (cohorts 3-6) was coated in individual aluminum trays via a solvent evaporation procedure. The particulate-containing polymer film was ground prior to release. Theoretical amounts of tobramycin applied to each sample were calculated based on the weight of coating applied to the allograft bone material and the percent of tobramycin included in the formulation. Tobramycin was released from the polymer coating on each cohort into PBS. PBS was sampled at designated time points and replaced to simulate sink conditions. For the data depicted in FIGS. 13A, 14A, 15A, 16A, and 17A, the tobramycin content in the PBS “release media” was assessed at each time point via a 96-well fluorescent assay

Release kinetics. Tobramycin is very water-soluble and thermostable during formulation as evidenced by no loss in bioactivity after included in a PCL formulation (data not shown) (Mousset et al., Int Orthop, 19 (1995): 157-161). However, detection of this small molecule aminoglycoside antimicrobial in a sample is complicated by lack of a unique optical signature. Therefore, tobramycin was derivatized with o-phthaldehyde (OPA) (Sevy et al., Biomed Sci Instrum, 46 (2010): 136-141). This reaction yields a chromophore by chemically coupling with primary amines on the drug, producing fluorescence signals with a dynamic range from 0 to 8 mg/ml and a limit of detection of 62.5 ug/ml (Sevy et al., Biomed Sci Instrum, 46 (2010): 136-141). The OPA derivatization reaction was verified via HPLC detection of tobramycin in the presence or absence of OPA (data not shown). In the absence of OPA, tobramycin did not elicit any absorbance or fluorescence signal. Furthermore, inherent OPA fluorescence was not detected in the absence of tobramycin. Thus, release of tobramycin from a variety of polymer formulations was compared using an OPA derivatization in a 96-well assay format as previously reported and validated by mass spectrometry (Sevy et al., Biomed Sci Instrum, 46 (2010): 136-141). To facilitate comparison, the measured amount of drug released at each time point was normalized, in a cohort specific manner, by the average (n=3-9) of the detected tobramycin after complete dissolution of the polymer coating in chloroform and subsequent extraction of tobramycin from the chloroform polymer solution with water. This amount was assumed to be 100% of the tobramycin added to the polymer coating system (100% release). The phase extraction procedure was controlled by determining the percent recovery of both free drug (approximately 100% recovery) and also microencapsulated (approximately 82% recovery) tobramycin. Thus, for microencapsulated samples, 18% of the amount of the 100% drug release sample was added and this value was used for all subsequent calculations. Moreover, the validated coating-dissolution, phase-extraction method was applied to time course release samples, allowing determination of the mass balance. After 8 weeks of release into PBS, between 97-100% of the drug was recovered from a PCL-tobramycin coating (data not shown).

As shown in FIGS. 13A, 14A, 15A, 16A, and 17A, the combination of fluorescent detection and mathematical validation provided accurate release kinetics and revealed significant differences among the different cohorts. FIGS. 13B, 14B, 15B, 16B, and 17B show the ZOI bioactivity data corresponding to the kinetic data shown in the FIGS. 13A, 14A, 15A, 16A, and 17A, respectively. The non-limiting bone implants shown in these figures are described in detail below. Impact of Bone Graft Carrier on Drug Release Kinetics. Drug loading and coating consistency relies on the polymer formulation, application technique, and the nature of the underlying bone graft substrate. Since the cancellous allograft bone generally arrives in two forms—namely croutons (i.e., fragments) and particulate (i.e., the granules at the bottom of the bag), the drug release rate and ZOI rates were compared. To investigate the influence of allograft substrate form (crouton or micro-size particulate), cancellous allograft fragments were weighed and coated with PCL (60 mg/ml in acetone) containing tobramycin (10% w/w) via dip-coating (i.e., dipping the crouton in the tobramycin-containing PCL solution) with vacuum drying (cohort 1). Approximately 20 mg of tobramycin-containing polymer coating was added to each fragment. Alternatively, 100 mg of micron-size particulate was weighed and coated with the same PCL tobramycin formulation by “solvent casting”—namely the tobramycin-containing PCL solution was applied over the top of the particulates and the solvent allowed to evaporate in individual aluminum trays leaving the behind the antibiotic: polymer coated individual allograft bone particulates (cohort 3). Allograft particulate-containing, tobramycin-releasing PCL films were re-morselized prior to release into PBS.

As shown in FIG. 13A, micron-size allograft particulate (solid circles, dotted line) displayed a higher initial burst release when compared to drug-releasing, polymer-coated cancellous allograft fragments (open circles, solid line). This may be an unintended consequence of including additional drug-releasing polymer not adhered to the allograft particulate or of the process used to re-morsalize the particulate after coating. To minimize the impact of the re-morselization process, coated micron-sized allograft particulate was not ground, but instead was crushed with a weighing spatula so as to mitigate the introduction of defects in the coating (i.e., cracks). The difference may also be attributed to discrepancies in the specific surface areas of the different allograft materials due to their different porosities. Cancellous allograft fragments are highly porous, but the porosity is not consistent from fragment to fragment, leading to differences not only in drug load but also in coating integrity, culminating in larger standard deviations. Large standard deviations preclude identification of significant differences in the tobramycin release kinetics arising from allograft fragments or micron-size particulate, particularly at later experimental time points. Allograft particulate also has an extremely high surface area, all of which is available for coating using the current technique; whereas, the internal porous structure of larger allograft bone may not be accessible to the coating.

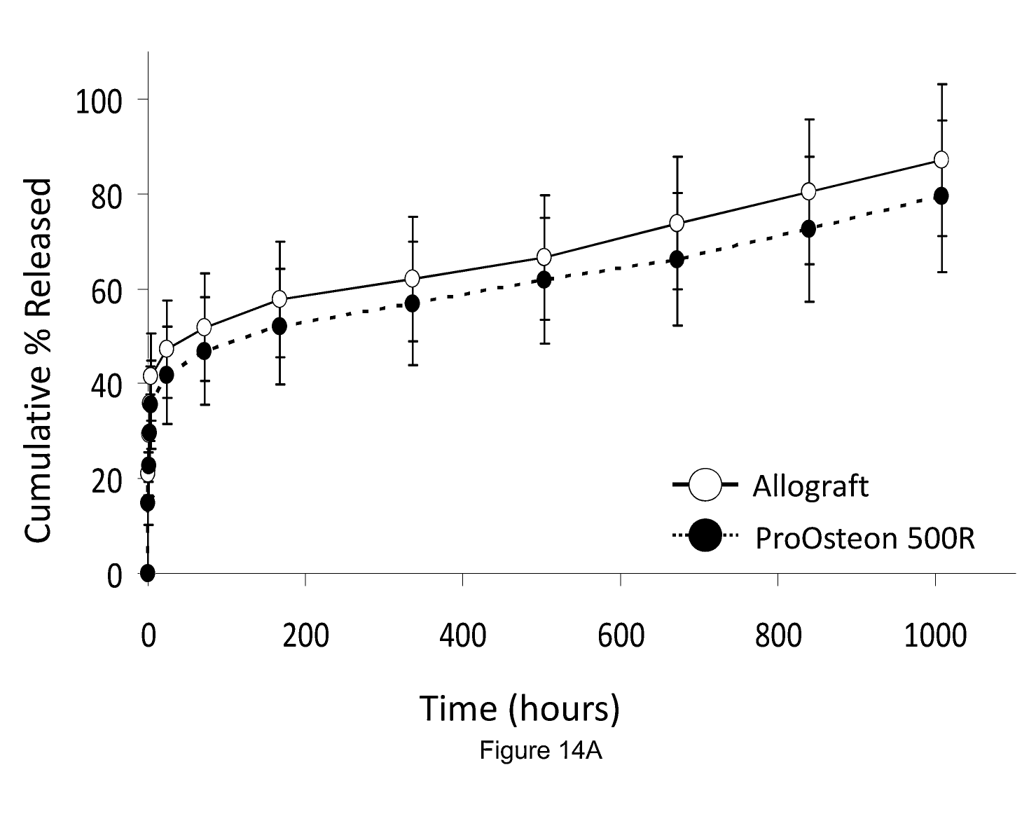

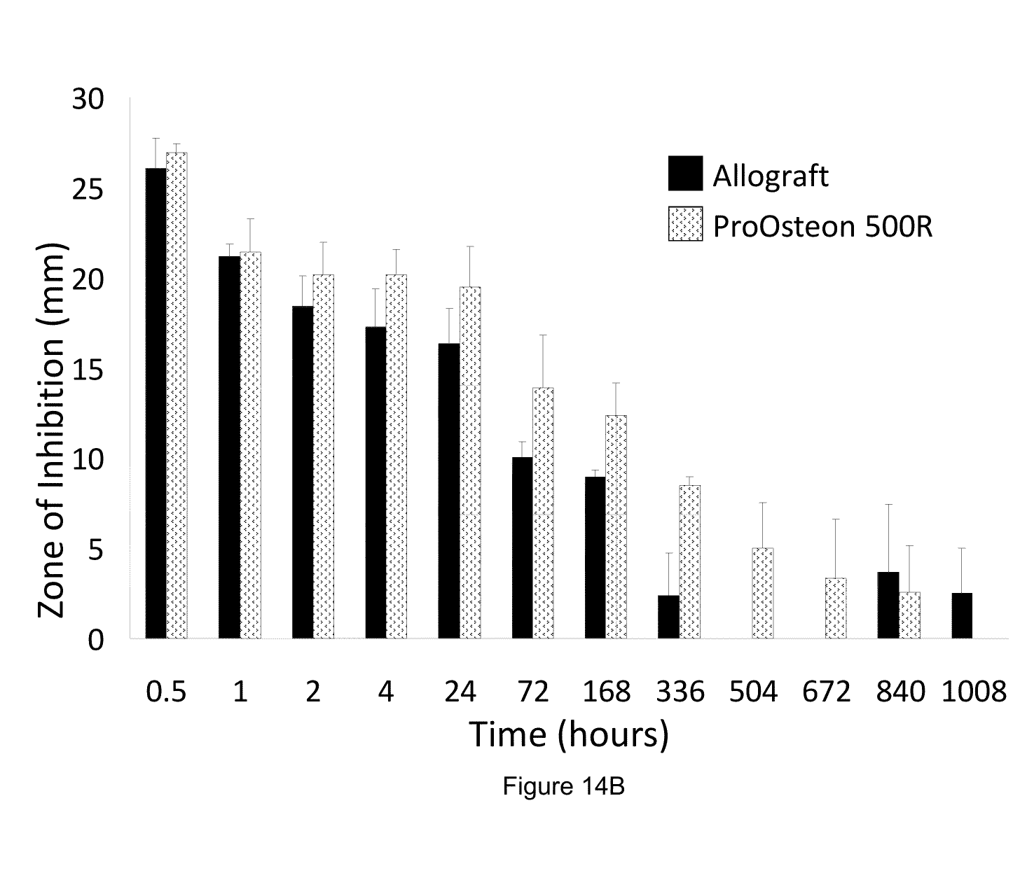

The superior release performance of coated allograft particulate led to its use in all subsequent experiments in this Example 1 with the notable exception of the synthetic bone graft filler, ProOsteon 500R® particulate, which were not micron-sized and had a porous structure reported similar to cancellous bone (pore size reported to be 280-770 um with 55% porosity; cancellous allograft pore size reported to average between 400-500 um with porosity ranging between 60-77% (Bloebaum et al, Clin Orthop Relat Res, (1994): 2-10). Not surprisingly, when ProOsteon 500R® fragments were dip-coated and vacuum dried analogously to the allograft fragments (cohort 1), tobramycin release did not differ significantly in its kinetics when compared to allograft coated crouton fragments (FIG. 14A), demonstrating the substrate independence of the drug-releasing coating system described on graft materials with similar microstructural features.

Impact of Coating Formulation on Tobramycin Release Kinetics.

In solution-based drug formulating and polymer vehicle coating, component compatibility issues in drug-solvent-polymer solubility, mutual miscibility and controlled solution stability are important design criteria (J. Liu et al, J. Pharm. Sci. 93: 132-143, 2004). Consideration of thermodynamics predictors of these properties (i.e., matching appropriate Hildebrand solubility parameters of the drug, polymer, and solvent(s), such as with PCL (δ (delta)=20.2 (Bordes et al., Int. J. Pharm. 383: 236-243, 2010) in acetone and tobramycin) facilitate sustained and controlled drug release and can help avoid phase separation (J. Liu et al., supra; Huang et al., J. of Applied Polymer Sci., 100: 2002-2009, 2006). Unfortunately, tobramycin is highly soluble in water and marginally soluble in alcohols while PCL is insoluble in water, highly soluble in chloroform, and soluble in acetone at elevated temperature. The dissimilarity in component solubility (i.e., powdered tobramycin added to acetone solvated PCL creates a tobramycin suspension upon mixing) and resulting solution heterogeneity and possible phase separation as a drug delivery system will impact the kinetics of drug release.

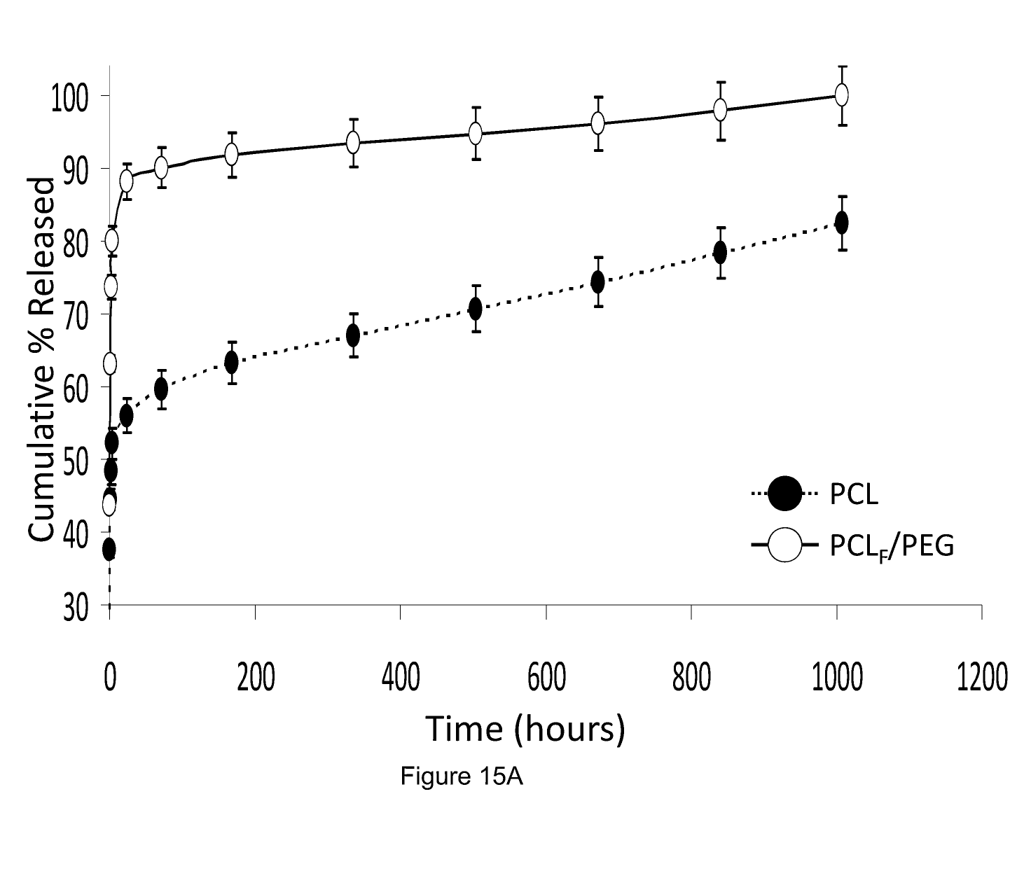

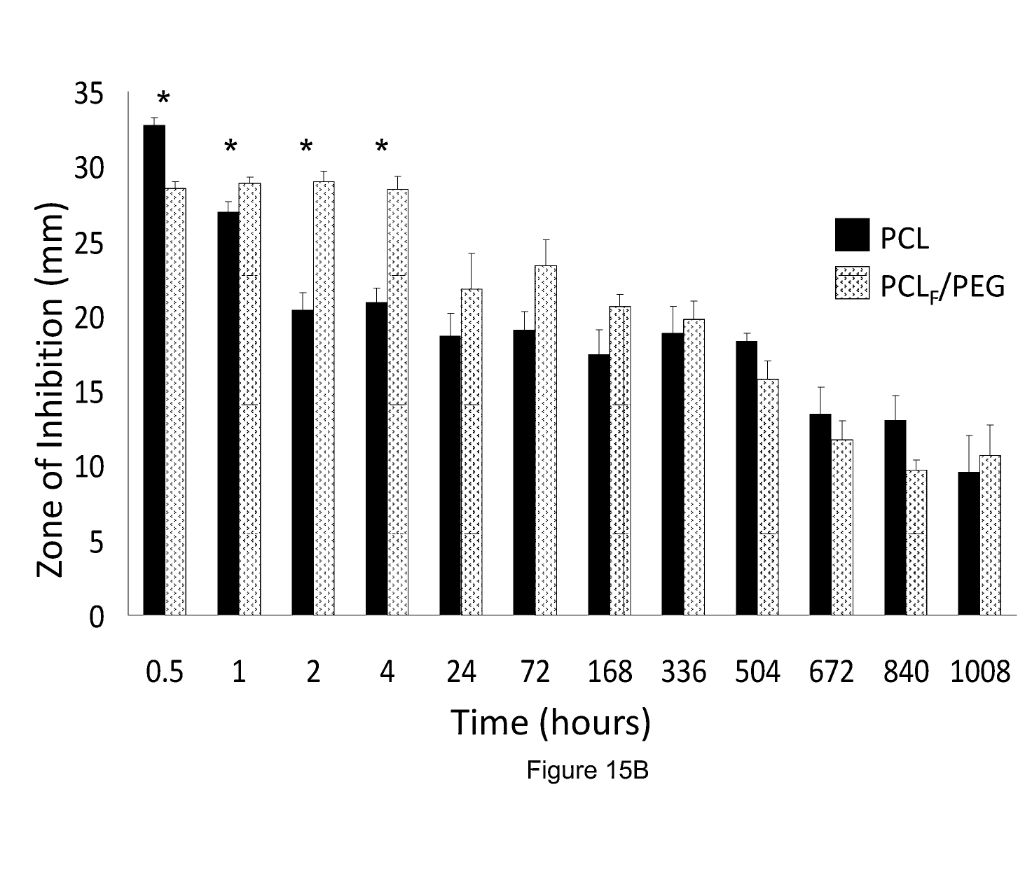

To limit the tobramycin burst release kinetics within the first 24 hours, potentially due to phase separation, some coating formulations were modified to include a 45% w/v aqueous polyethylene glycol (PEG δ (delta)=22.9 (J. Liu et al., supra) 20 kD) feed solution (cohort 4), which would, in theory, retain tobramycin in a more compatible aqueous phase thereby mitigating phase separation and creating a more homogeneous polymer-drug formulation for coating (see FIGS. 15A-15B). For these bone implants, PEG was added to PCL and tobramycin. The interaction of PCL and PEG are well studied, indicating that upon molten mixing, the structure of the blend is dictated by a balance between liquid-liquid phase separation and polymer blend crystallization. However, in solution, miscibility can be hampered by mismatched component Hildebrand values (dispersion, polar, hydrogen bonding (Wu et al., J. Pharm. Sci. 71: 1285-1287, 1982) Bordes et al, Int J Pharm, vol. 383, pp. 236-43, 2010; Liu et al., J Pharm Sci, vol. 93, pp. 132-43, 2004; Huang et al., Journal of Applied Polymer Science, vol. 100, pp. 2002-2009, 2006); furthermore, the solvents for each polymer are different (PCL-acetone, PEG-water). Thus, one challenge in generating the implants described herein was the difference in solubility between the PCL polymer (soluble in acetone but not water) and the drug (soluble in water but not acetone). This causes the drug to want to segregate from the polymer when coating the croutons, an effect known as phase separation. PEG, which is also soluble in water, was included in the formulation shown in FIGS. 15A and 15B to see if its presence could enhance the solubility of tobramycin in the formulation and change the release kinetics. The “F” (free) subscript on PCL in FIGS. 15A and 15B indicates that tobramycin was used free and was not itself encapsulated in a polymer (i.e., the tobramycin was free tobramycin, not micro encapsulated tobramycin).

As shown in FIG. 15A for cohort 3 and cohort 4, although this formulation did modify the tobramycin release kinetics, it resulted in a greater amount of tobramycin being released not only within the first 24 hours (burst), but also over the entire course of the experiment. Nevertheless, the bioactivity of the released antibiotic shows insignificant differences between the two formulations (FIG. 15B). Additionally, FIG. 15A shows that dramatic alterations in the coating solvent system improved the antibiotic solubility in the polymer formulation to allow greater than 95% of the drug to be recovered from the PCL (free tobramycin)/PEG coated implant over a 6-week time course of release. Previous reports suggested that inclusion of PEG in a PCL polymer formulation might accelerate drug release (Wan et al., Biomacromolecules 7: 1362-1372, 2006); however, in the system described herein, inclusion of PEG in the formulation improves the miscibility of tobramycin with PCL by allowing the drug to be retained at the delivery substrate for a longer duration due to polymer blend crystallization (see also Bramfeldt et al., Polm. Degrad. Stab. 93: 877-882, 2008). Furthermore, certain formulations may be capable of releasing antibiotic for 8 weeks or more (data not shown). This extended duration of release provides an important performance distinction over other commercially available antibiotic-releasing implantable bone delivery systems such as polymethylmethacrylate (PMMA) cements that suffer early burst releases followed by low-level, and incomplete antibiotic leaching inadequate to provide longer term efficacious antimicrobial protection (Kanellakopoulou and Giamarellos-Bourboulis, supra; Diefenbeck et al., Injury, 37 Suppl 2 (2006) S95-104; Chang, J Control Release, 110 (2006) 414-421). Importantly, unlike native or biodegradable synthetic bone grafts capable of both osteoclast remodeling and osteoinductivity to form osteocytes, PMMA acts as a permanent foreign body, providing a substrate for bacterial adhesion and biofilm development after antibiotic release exhaustion, promoting both secondary infections and resistant bacteria (Neut et al., J Antimicrob Chemother, 47 (2001): 885-891); Chang et al., J Control Release, vol. 110, pp. 414-21, 2006, Diefenbeck et al., Injury, vol. 37 Suppl 2, pp. S95-104, 2006.

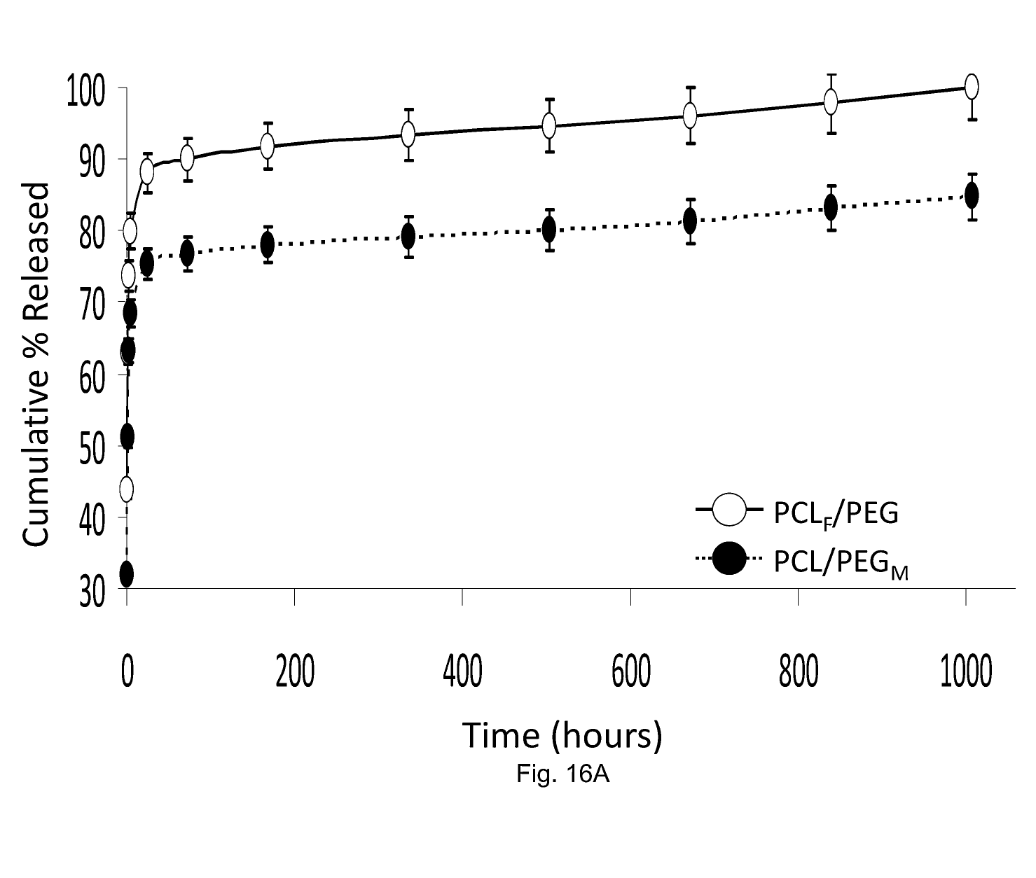

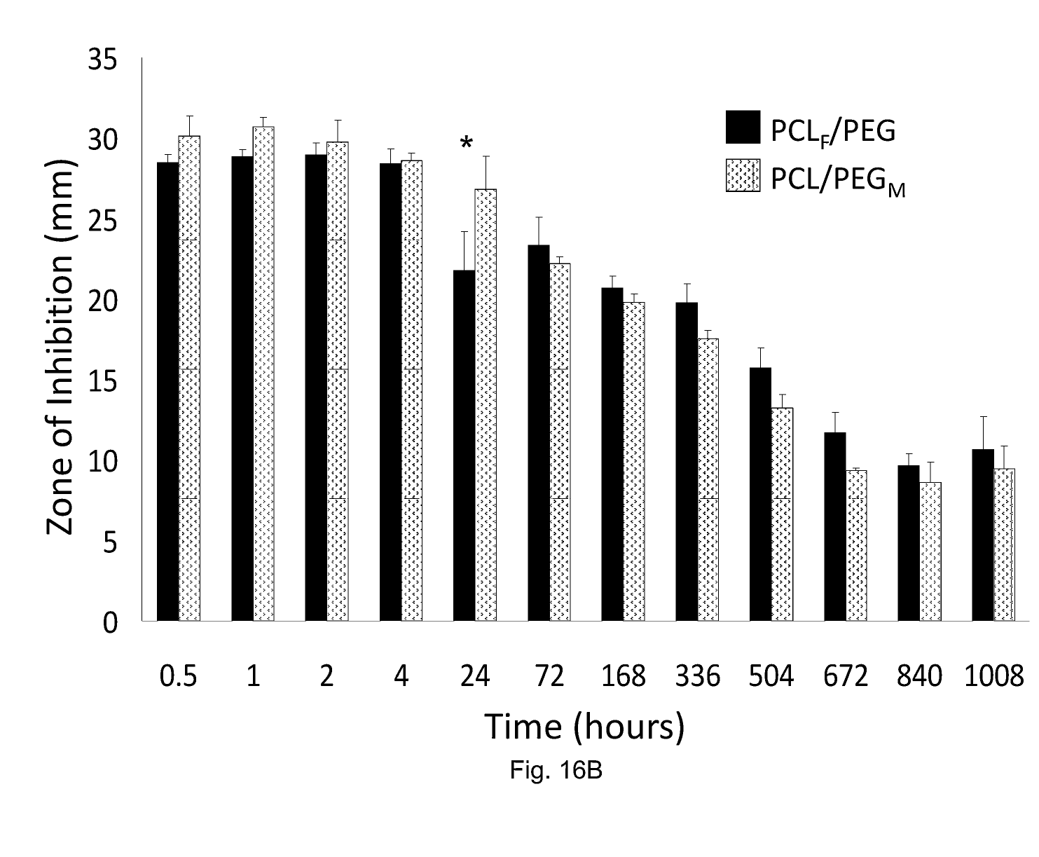

Tobramycin drug release kinetics were further modified using commercially microencapsulated tobramycin to further slow and extend the duration of drug release. The modifications of the fabrication method and the implants resulting therefrom are shown in FIGS. 9A-9C. Microencapsulated tobramycin was stirred into the 45% w/v aqueous PEG feed solution which was then further mixed with a PCL-acetone-free tobramycin solution prior to coating (cohort 5; labeled as PCL/PEG_{M} in FIGS. 16A and 16B). The theoretical amount of tobramycin in the final formulation was equivalent to that formulated with 45% w/v aqueous PEG solution mixed into PCL-acetone (1:1) with free tobramycin only (cohort 4; labeled PCL_{F}/PEG in FIGS. 16A and 16B; and also depicted schematically in FIG. 9B). The drug release kinetics from the two formulations mirror each other with those produced from the microencapsulated formulation (cohort 5; black circles dotted line in FIG. 16A) being delayed releasing approximately 10% less tobramycin at each time point when compared to release of free tobramycin (cohort 4; open circles, solid line in FIG. 16A). The ZOI bioactivity data of cohort 4 and cohort 5 are relatively similar (see FIG. 16B). This experiment is extended and may reveal small secondary burst release of antibiotic, which would present a significant and impactful advance, particularly for biofilm persistor cells (e.g., that are senescent) thought to support recurring osteomyelitis (Costerton, J. W., Rev. Infect. Dis. 6: 608-616, 1984; Marriot et al., Curr Opin Investig Drugs, 8 (2007): 887-8981 Nelson et al., Clin Orthop Relat Res, (2005): 25-30).

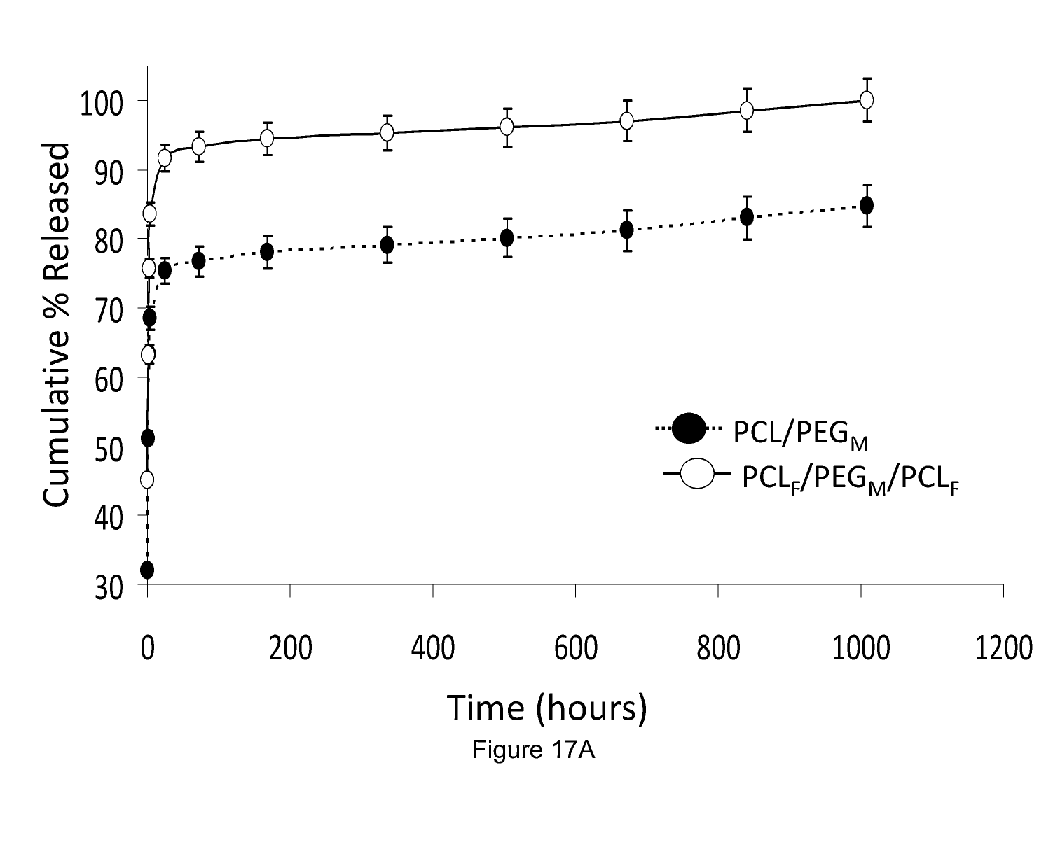

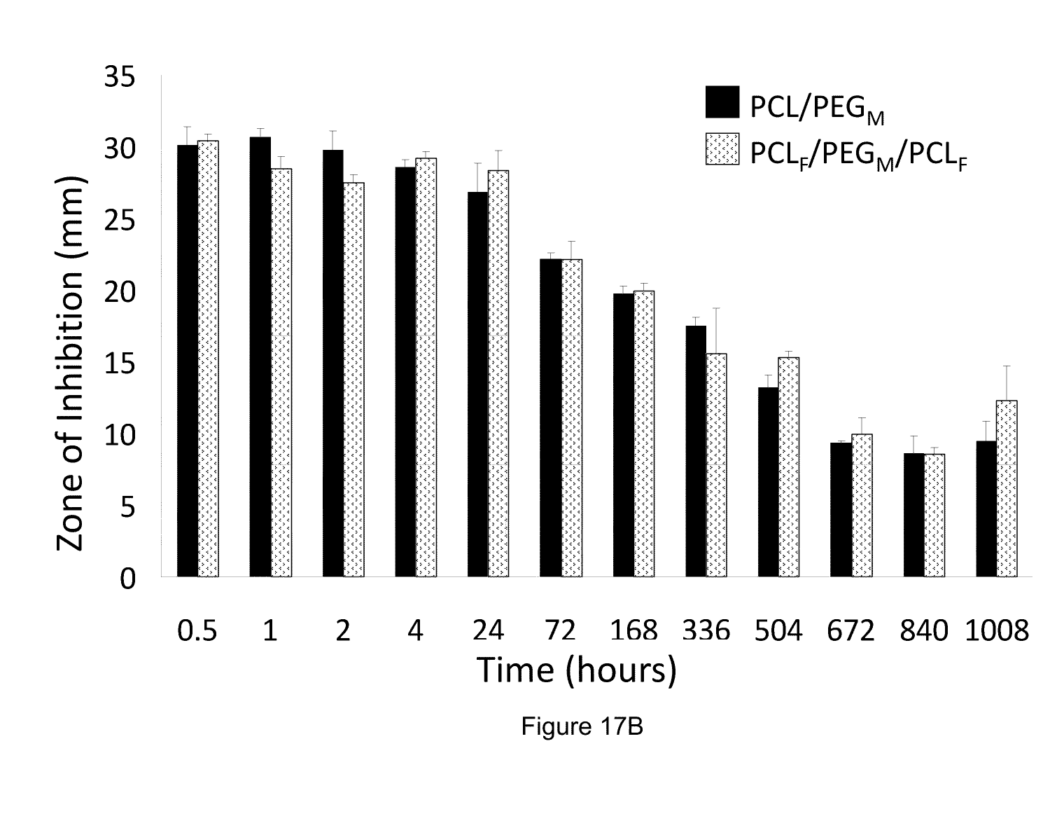

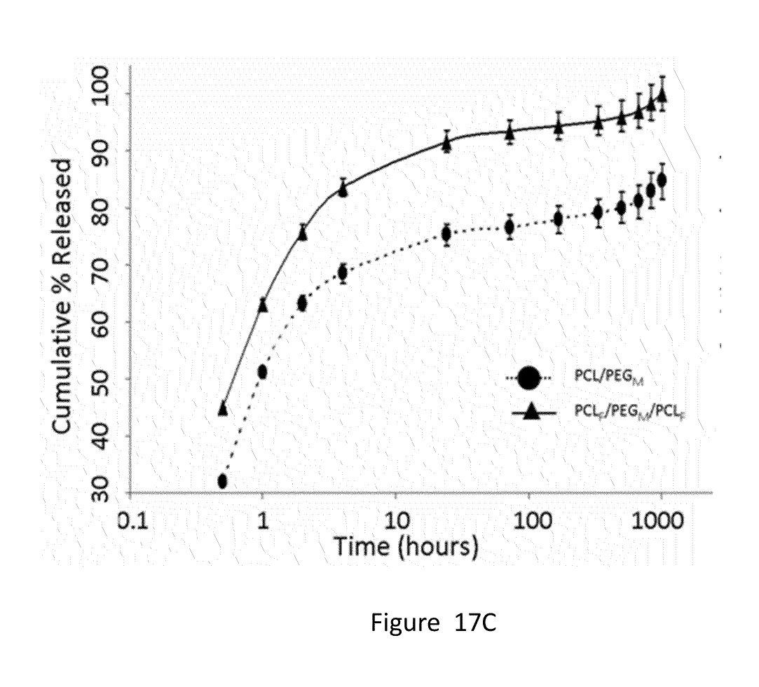

Since the addition of microencapsulated tobramycin into a PEG/PCL-based coating formulation retarded the tobramycin release kinetics, the influence of the coating application technique was also investigated (see FIGS. 17A and 17B). Microencapsulated tobramycin was suspended in a 45% aqueous PEG solution while free tobramycin was suspended in the PCL-acetone solution. These two suspensions were treated as independent formulations and applied to allograft particulate material as layers. Each layer was allowed to dry at ambient temperatures overnight and the solid film remorselized prior to applying the next layer. The entire multi-layer drug-releasing polymer film encasing the allograft particulate was crushed and suspended in PBS as a release medium. Notably, solvent removal was deemed to be complete once the weight of the encapsulated allograft bone filler was stable. Based on the immiscibility of formulations A and B, their alternate layering should not have dissolved each previous underlying layer. Interestingly, release kinetics from this layer-by-layer coating (cohort 6; labeled PCL_{F}/PEG_{M}/PCL_{F} in FIGS. 17A-17C) and its analogous composition directly mixed but unlayered formulation (cohort 5; labeled PCL/PEG_{M} in FIGS. 17A-17C), when compared to the PCL:PEG unencapsulated formulation (cohort 4; labeled PCL_{F}/PEG in FIGS. 16A and 16B), are all dominated by the free tobramycin fraction (compare FIG. 17A to FIG. 16A) with very little differences in release kinetics propagated over the 6 week time course beyond that initially seen in the first 30 minutes' burst (see FIG. 17C, with cohort 5 as solid circles and cohort 6 as solid triangles).

Regardless of coating formulation, antimicrobial activity was confirmed via in vitro bacteriostatic assays based on a modification of the standard techniques for determining the minimal inhibitory concentration (MIC) for an antibiotic (data not shown), as well as classic zone of inhibition or radial diffusion assays (see FIGS. 13B, 14B, 15B, 16B, and 17B). Maintenance of local antibiotic concentration exceeding the MIC is critical over the course of both acute and chronic therapy to rapidly kill viable pathogens (e.g., bacteria) and prevent development of resistant bacteria, particularly in compromised, infection-susceptible bone with limited perfusion and antibiotic penetration. Disparate sensitivity of the ZOI assays and the fluorescent tobramycin detection assay are such that even under zero order release kinetics, a zone of inhibition can still observed. Thus, direct comparisons of ZOI-based antimicrobial activity with drug release kinetics are obscured in conjecture; only general trends can be inferred. All of the coating cohorts investigated provided antimicrobial activity throughout the clinically relevant duration of the assay (6 weeks), although the crouton allograft fragment-based cohorts temporarily failed to exhibit antimicrobial activity as indicated by the absence of a clear ZOI (see black bars in FIGS. 13B and 14B at 504 and 572 hours post-implantation).

Tobramycin release was primarily affected by the allograft material morphology (larger porous crouton fragments or micron-sized porous particulate, see FIG. 13B), showing significantly greater antimicrobial activity from coated micro-sized porous particulate. This translates to a higher amount of released tobramycin at several time points (½ hour, 1 hour, 72-840 hours). The cancellous allograft fragment cohort (black bars in FIG. 13B) displayed larger standard deviations, particularly at the later time points where only one or two samples of the cohort were still exhibiting antimicrobial activity, falling short of the desired therapeutic window. Despite being weight matched and normalized, large standard deviations also plagued direct comparison of allograft fragments with ProOsteon 500R® synthetic fragments, with significant differences identified only between 4 hours and 1 week (FIG. 14B). Conversely, consistent antimicrobial activity out to 6 weeks (1008 hours) was observed for coated allograft particulate substrates (dotted white bars in FIG. 13B), indicating that sufficient drug load may still remain for microbial killing past the 6-week experimental time frame. This may be indirectly attributed to the significant differences in coating application technique (dip-coating versus solvent casting on the two allograft materials) dependent on the substrate physical form. Nevertheless, micron-sized allograft particulate material may provide more efficient packing into avascular dead spaces often prevalent in injured and surgically repaired bone defects and higher graft packing density to provide enhanced duration of antibacterial efficacy in vivo when compared to larger coated allograft or synthetic bone graft fragments, keeping in mind that graft density must be controlled to allow proper bone metabolism (Mailinin et al., Open Orthop J. 1: 19-24, 2007).

In addition to drug release kinetics dependence on the geometry of the underlying substrate, the polymer formulation can also be engineered to alter the rate of drug release. Based on the disparate release kinetics measured upon addition of the 45% PEG aqueous solution (cohort 4; PCLF/PEG in FIG. 15A) to the PCL base polymer formulation in acetone (PCL in FIG. 15A), resulting antimicrobial activity was predicted to also differ. However, ZOI measurements demonstrated significant differences only between 30 minutes and 4 hours, despite relatively small standard deviations (see FIG. 15B). Both formulations released tobramycin amounts sufficient to produce a ZOI throughout the entire 6-week time-course. Inclusion of microencapsulated tobramycin in the coating formulation also did not produce significant differences in amounts of active drug released (FIG. 16B), despite slightly slowing antibiotic release (FIG. 16A). Moreover, differences in tobramycin release kinetics from analogous formulation cohorts (i.e., cohorts 5 and 6) prepared using different application techniques were dominated by the outermost PCL layer containing free tobramycin as indicated by a lack of significant differences (compare FIGS. 16A and 17A). As such, antimicrobial activity was predicted to also be very similar (FIG. 16B) and was robust for the 6-weeks' duration, with no statistical differences between the ZOI obtained from cohort 5 and cohort 6 (see FIG. 17B) and virtually indistinguishable from that obtained from cohort 4 (compare FIG. 17B to FIG. 16B). Maintenance of antibiotic tobramycin drug concentrations throughout the 6-week experimental duration represents a significant advancement over other FDA-approved available polymer-controlled, locally antibiotic-releasing orthopedic implants. Most of these not only quickly become pharmaceutically silent due to inadequate drug release kinetics but also act as foreign bodies, facilitating infection, and ultimately promoting antibiotic resistance (Diefenbeck et al., supra; Neut et al., supra).

Thus, from this Example 1, the degradable polymer-controlled, antibiotic-releasing bone graft system described was shown to be able to successfully deliver tobramycin antibiotic in vitro over 6 weeks, offering a distinct performance advantage over current antibiotic-releasing technologies for bone that may inadvertently promote both infection and bacterial antibiotic resistance. Furthermore, the broad implications of polymer-mediated control over local drug release kinetics with some degree of versatility presents an attractive alternative technique for improved local delivery of different classes of bioactive molecules from tissue implants, particularly in a diffusion-limited tissue such as bone defects. A facile, convenient drug fluorescence assay was developed to evaluate drug release kinetics from a variety of tobramycin-loaded PCL-coated bone graft fillers. ZOI assays confirmed the antimicrobial activity of tobramycin after coating formulation and release, independent of the underlying graft substrate or coating method (see FIGS. 13B-17B).

Regardless of the polymer matrix formulation, micron-sized allograft bone particulate provided the most desirable release profile for tobramcyin. Derivative to its small size and high porosity, micronized allograft and/or synthetic graft may also provide a more efficacious wound packing material to prevent the formation of inadvertent avascular dead spaces, as opposed to larger porous fragments (see FIGS. 13A and 13B). In fact, several allograft particulate-based cohort formulations yielded distinct ZOIs throughout the 6-week study duration, indicating an potentially longer window of therapeutic drug release mediated by polymer degradation, as opposed to drug leaching from coating defects and barrier inconsistencies. This assertion is supported by data on mass balance of the coating system after 8 weeks of release in vitro (see Example 2 below). Surprisingly, incorporation of microencapsulated tobramycin did not alter the rate of tobramycin release (shape of the kinetic curve remained unchanged) or the resulting antimicrobial activity and exhibited only a limited capacity to slow the amount of tobramycin released over the 6-week study duration (see FIGS. 16A and 16B). Addition of concentrated aqueous PEG solutions to the PCL-acetone-drug formulation dramatically altered tobramycin release kinetics but did not exhibit any analogous extensions of tobramycin efficacy (i.e., ZOIs) (see FIGS. 15A and 15B). While this observation is attributed to the different sensitivities of the respective assays used, this alteration in tobramycin release kinetics provides some indication of the versatile extended drug release modulation possible with this coated bone graft implant system. Blending PEG with PCL may change coating morphology to increase the physical state of suspended drug in the coating and subsequent rate of tobramycin release from the allograft bone.

### Example 2

Ideal antibiotic delivery systems would provide killing via a burst release (i.e., a bolus release) within the first 24-hour period, after administration followed by a sustained release above the minimal inhibitory concentration (MIC) to address the remaining microbial threat out to the 6-week time point (previously established by the orthopedic community as important to infection prevention) (Kanellakopoulou and Giamarellos-Bourboulis, supra; Chang et al., J Control Release 2006; 110: 414-21). Targeted antibiotic delivery sustained above the MIC minimizes the selective pressures of antibiotic resistant infections (Patzakis and Zalavras. J Am Acad Orthop Surg 2005; 13: 417-27; Strachan C J., J Antimicrob Chemother 1993; 31 SupplB: 65-78). However, current methods of simple physical absorption of drug to allograft bone can result in only a bolus release (Kanellakopoulou and Giamarellos-Bourboulis, supra), with no sustained release to combat further infections. Polymer-controlled antibiotic release from allograft bone material is a desirable alternative as it allows for long-term antibiotic success.

The use of a pharmaceutical-encapsulating, rate-controlling polymer membrane with defined degradation character to endow allograft bone with antimicrobial activity provides a level of delivery control unattainable with mere physical adsorption. Although polymer-controlled local drug delivery is not a new idea, the successful tailoring of the polymer coating to provide predictable drug release kinetics provides an important twist on a classic idea (Davidoff et al., Biomed Sci Instrum 2010; 46: 184-9; Davidoff et al., Biomed Sci Instrum 47:46-51, 2011; Sevy et al., supra). Previous studies have demonstrated changes in the release kinetic profile of tobramycin from this polymer-controlled drug delivery system based on 1) a change in the molecular weight of the polymer, 2) the incorporation of a water, non-solvent (Davidoff et al., Biomed Sci Instrum 2011; 47:46-51, 2011) or 3) the addition of an aqueous polyethylene glycol (PEG) solution (see Example 1 above) in the coating fabrication processes. The most significant and beneficial alterations in tobramycin release were noted when the miscibility of the drug and the polymer were changed as a function of solvent, most likely as a result of improved drug solubility, hydrophobicity, and charge. To investigate the effect of solubility and miscibility on drug-release profile, water-soluble antibiotics (vancomycin, oxacillin) and non-water soluble antibiotics (ciprofloxacin, rifampicin) were released from a polymer-controlled releasing membrane with and without the incorporation of a water, non-solvent into the formulation. Moreover, the danger of multi-drug resistant pathogens will be investigated by considering the combinatorial therapeutic efficacy of ciprofloxacin, rifampicin, and vancomycin against S. aureus as released from a local drug delivery system using a polymer-controlled releasing membrane coated onto allograft bone. Tailoring the release kinetics as a function of antibiotic solubility should provide clinicians with a long-term, antibiotic delivery system that can be customized and combined to fit each patient's needs while concurrently mitigating the development of antibiotic resistance.

By developing a local drug delivery system that controls the release of antibiotic via a degradable polycaprolactone (PCL) polymer membrane coated on an implantable allograft bone delivery vehicle, an increase in the bioactive longevity of antibiotic therapy is anticipated. Furthermore, combinatorial antibiotic experimentation will provide clinicians with an “ala carte” style of antibiotic treatment that can be tailored to meet each patient's needs while mitigating the development of bacterial resistance due to systemic and prolonged antibiotic overuse and poor patient compliance.

In this example, another non-limiting implant was fabricated and tested for its ability to diffuse drug for a prolonged amount of time. For this example, the following methods were used.

Fabrication of Antimicrobial Allograft Bone Fragments (AABF). Cancellous allograft bone fragments (Miami Tissue Bank) were weighed and similar weights were selected for each cohort. Each cohort was coated with a drug-releasing polymer to investigate release kinetics of different antibiotic-containing formulations (n=6). Each delivery system consisted of allograft bone coated with 18-22 mg of a 60 mg/mL polycaprolactone (PCL, Sigma CAS 24980-41-24, St. Louis, Mo., USA) solution containing 16 mg/mL antibiotic. PCL was resuspended in acetone at 45° C. Subsequently, antibiotic powder was added to PCL solution for cohorts without the water non-solvent. Alternatively, for cohorts containing water non-solvent, water was added to the PCL acetone solution at 4% (v/v) prior to adding the antibiotic powder. The cohorts differed according to 1) which of the antibiotics was incorporated (vancomycin HCl, rifampicin, ciprofloxacin, oxacillin, and ciprofloxacin HCl) and 2) the addition of 4% water, non-solvent to the system. Standards containing only one drug were evaluated as a baseline to determine combinatorial effects.

Release Studies. The incorporated antibiotic was subsequently released from each cohort via incubation in 5 mL of phosphate buffered saline (PBS, cat#BP661-10, Fisher Scientific) at 37° C. Release media was collected and completely exchanged at various time points between 24 hours and 8 weeks. Subsequently, 500 uL of each collected release media was dried in a concentrator and stored at 4° C. for microbial studies.

Scanning Electron Microscopy (SEM). Three fragments from each cohort were analyzed for surface characteristics using low vacuum SEM (FEI Quanta 3D dbFIB). Microanalysis system software displayed the real-time, back-scatter electron detector (BSED) images captured from the microscope and allowed the capture of images between 1 mm and 100 um magnification.

Kinetics Assay. Dried antibiotic release samples were reconstituted in 96-well plates with 100 μL of PBS. An absorbance assay (320 nm) was used to determine the relative concentration of antibiotic in each sample for ciprofloxacin (salt and free-base form), rifampicin, and vancomycin HCl. Plates with rifampicin or ciprofloxacin release media were read for absorbance (320 nm) using a microplate reader (BioTek spectrophotometer and Gen5 1.09 software). Vancomycin containing samples were tested using 2 uL of the reconstituted sample read in a BioTek Take3 quartz plate using the Gen5 1.09 software. Tobramycin sulfate samples were analyzed for comparison using a modified o-phthaldialdehyde (OPA)-based fluorescence assay (100 uL sample, 100 uL isopropanol, and 200 uL OPA reagent (Sigma P-05322)) and read on the Biotek microplate reader at 360 nm excitation and 460 nm emission (Sevy et al., Biomed Sci Instrum, vol. 46, pp. 136-41, 2010).

Zone of Inhibition. Immediately following the kinetics assay, filter paper disks (diameter=6 mm) were placed in the antibiotic release samples in the 96-well plate and dried overnight. Staphylococcus aureus (ATCC 25923 from American Type Culture Collection, Manassas, Va., USA) streaked Brain-Heart Infusion (BHI, BD cat#237500) agar plates were prepared for zone of inhibition experiments using pooled isolated colonies from blood agar plates diluted to a standard concentration (0.5 McFarland units). Subsequently, each drug-containing disk was placed on the bacterial-streaked agar 24 cm apart. Plates were incubated for 18 hours at 37° C. Electronic calipers were used to measure the cleared (no bacterial growth) diameter surrounding each disk (zone of inhibition). If no measurable zone was present the diameter was recorded as zero.

Combination Therapy. Ciprofloxacin, rifampicin, and vancomycin HCl were evaluated in combination (ciprofloxacin:rifampicin, rifampicin:vancomycin, ciprofloxacin:vancomycin) by combining 100 ul of release media (i.e., media released from the implant) for each time point and testing the bioactivity using zone of inhibition, as described above. 100 uL of uncombined release medias were used as controls. A student's 2-tailed t-test was used to determine if there was a significant increase in bioactivity when antibiotics were tested in combination.

In this Example, the following results were obtained.

Sample Fabrication. Polycaprolactone (PCL) (10 kD PCL, 60 mg/ml) polymer was dissolved in acetone at 45° C. Subsequently antibiotic (ciprofloxacin (salt and free-base forms), rifampicin, vancomycin HCl, oxacillin) and/or 4% water non-solvent was added to the solution to create the final coating formulation. Antibiotic polymer solutions were used to dip-coat cancellous allograft bone fragments (average dimensions 6 mm×5 mm×4.5 mm). Each antibiotic was added to two cohorts: one with a 4% water non-solvent in the formulation, and one without the water non-solvent component according to methods previously described (Davidoff et al., Biomed Sci Instrum, vol. 47: 46-51, 2011). The amount of antibiotic applied to each allograft was determined by using the weight of the applied coating and the antibiotic percent of the formulation. In past studies, tobramycin was used as the drug of choice due to its clinical relevance, high thermostability, and efficacy.

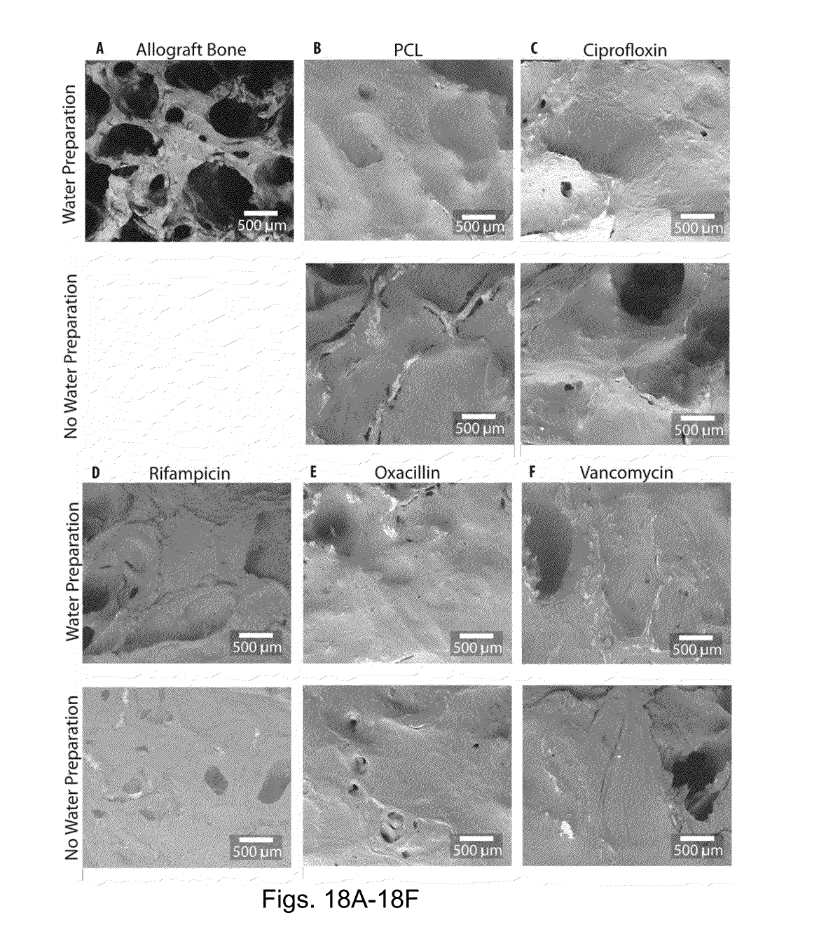

Scanning Electron Microscopy. In order to assess the PCL coating consistency and allograft surface coverage (i.e., to ensure a uniform mixture), the physical characteristics of the different surfaces were analyzed using SEM images (500 nM, 100 nM, and 20 nM). Images (500 nm) revealed that the polymer coatings of all antibiotics and coatings formulations with and without, water exhibit consistency and coverage (see the natural (allograft) croutons shown FIGS. 18A-18F; of course synthetic graft substrates could also have been coated). Furthermore, there were no noticeable physical characteristics that implied a difference in coating consistency between the various antibiotics and sample preparation methods. The implants shown in FIGS. 18A-18F were all dip-coated, vacuum-dried allograft croutons.

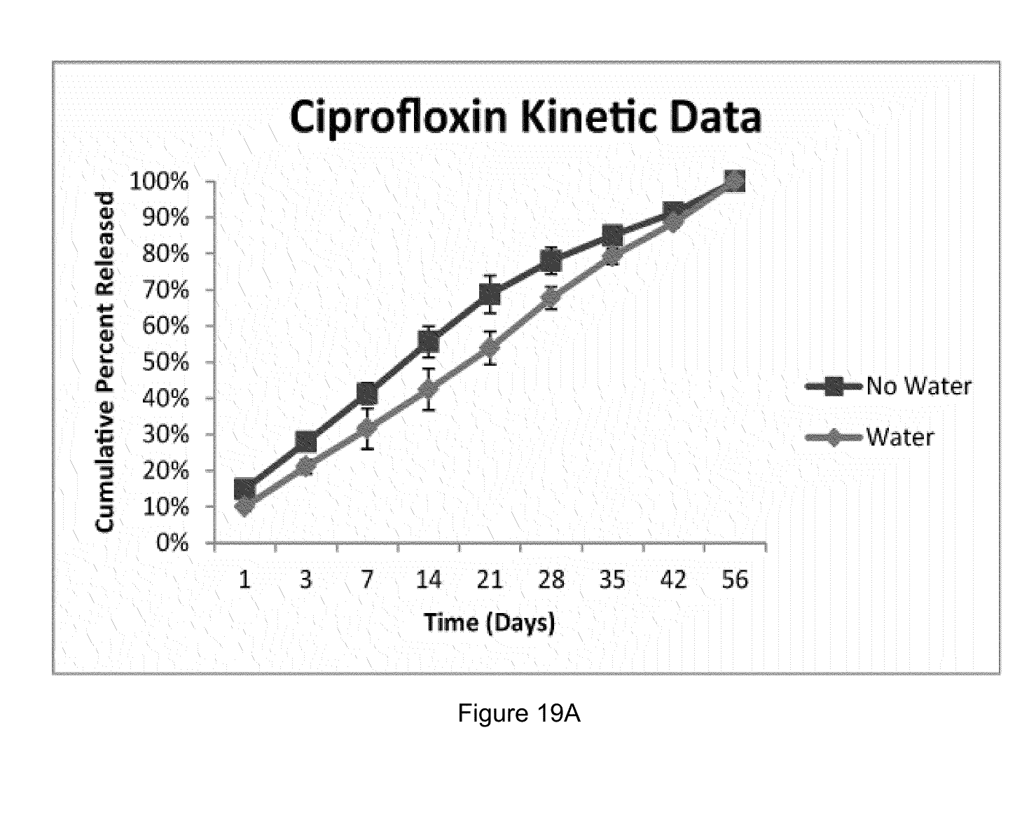

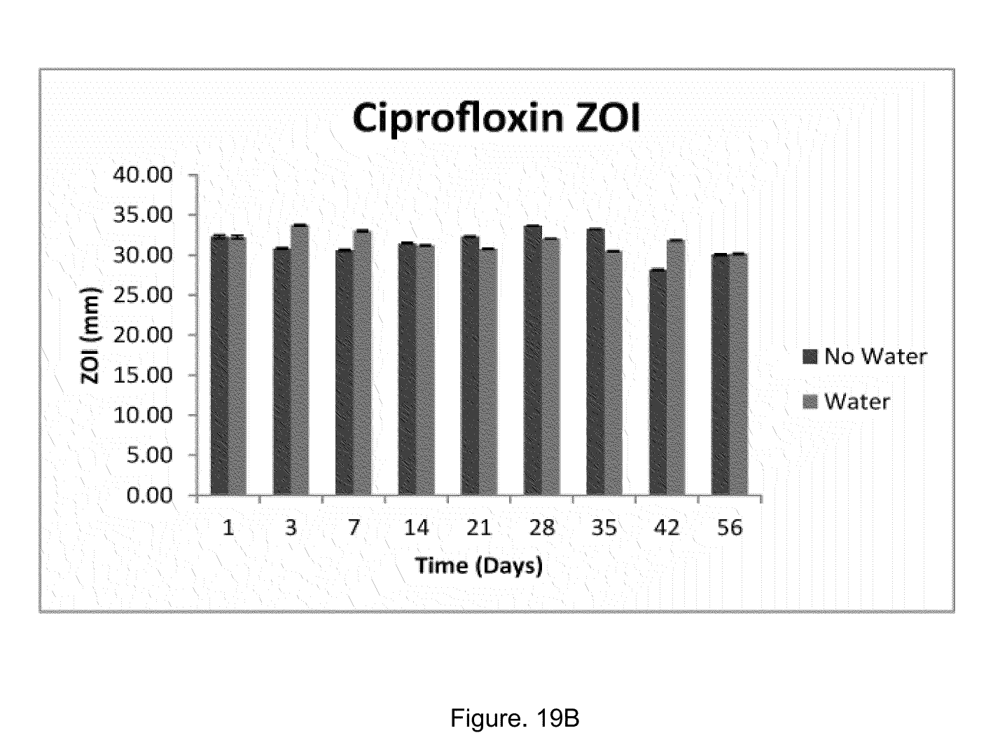

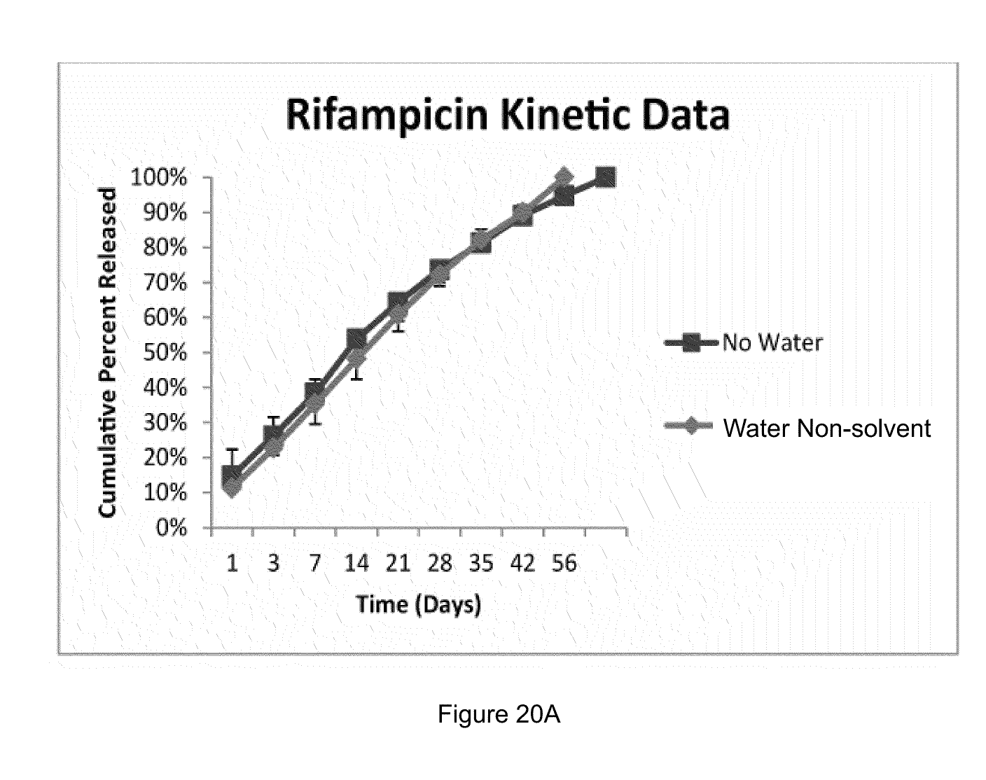

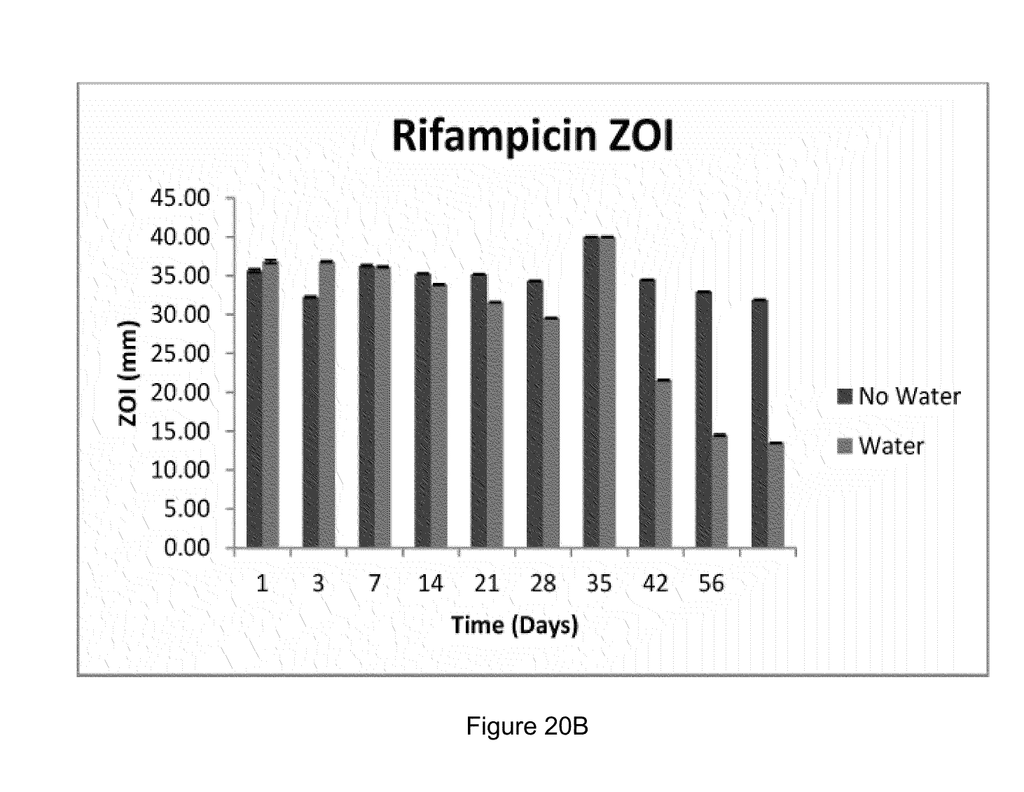

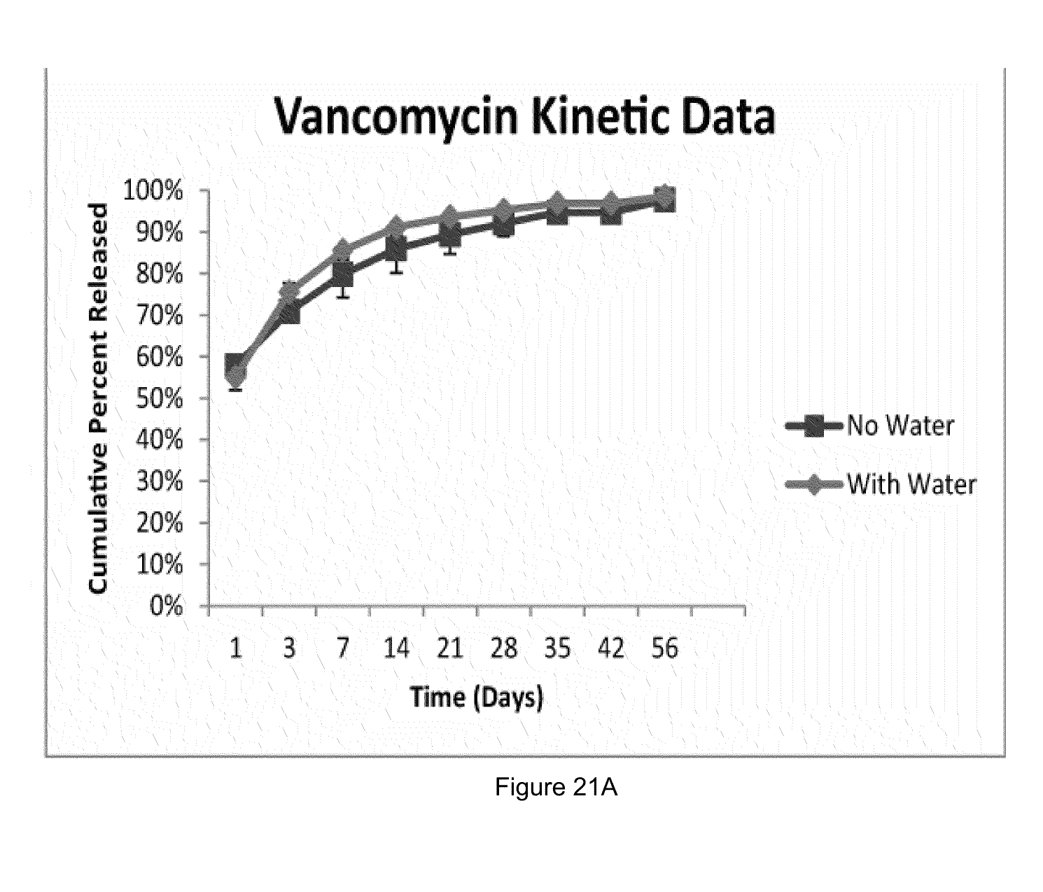

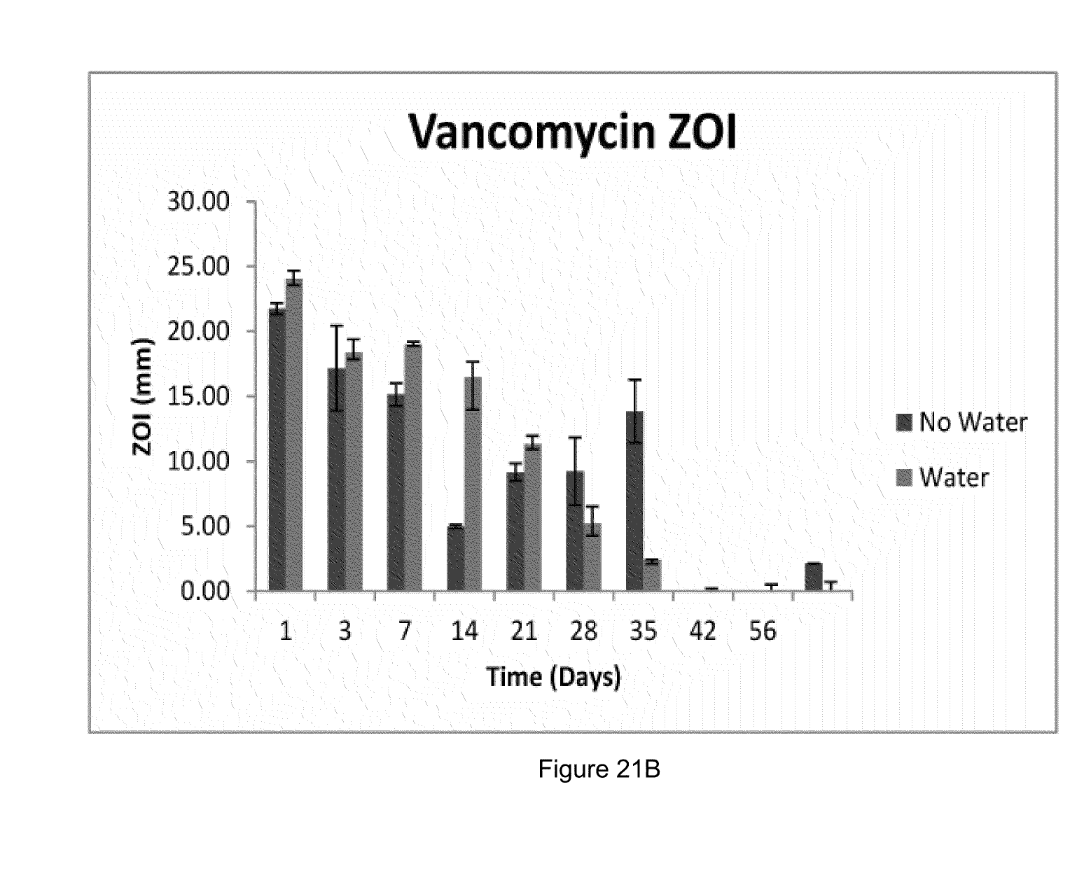

Release kinetics. Antibiotics were released from the polymer-coated allograft bone into PBS. PBS was completely removed replenished at regular time points out to 8 weeks in order to simulate sink conditions. A kinetic release curve for each antibiotic was calculated using a standard curve of the absorbance for each antibiotic and normalizing to the amount of antibiotic theoretically applied to each crouton based on the weight of the coating and the amount of antibiotic added to the formulation (see FIGS. 19A, 20A, and 21A). All antibiotics demonstrate a measurable amount of antibiotic in the release media throughout the experimental time course.

Table 2 is a chart showing drug and polymer solubility.

TABLE 2

| | Solubility |
|---|---|
| Drug | |
| Tobramycin Sulfate | water >>> acetone >> ethanol |
| Ciprofloxacin HCL | water >>> ethanol >> chloroform |
| Vancomycin HCL | water >>> chloroform & ethanol |
| Polymer | |
| Polycaprolactone | chloroform >> acetone >> ethanol |

In Table 2, note the discrepancy between the solubilities of the drugs and the polymer. This difference has a dramatic effect on the miscibility of the polymer and the drug and forms the basis for the drug release method described here.

Based on differential polymer/drug solubilities, phase extraction methods were used to separate and isolate the antibiotic, polymer, and allograft components of the system. Drug load was subsequently quantified using optical absorbance or fluorescence assays. Methods were validated based on control samples without polymer or bone graft, revealing over 85% drug recovery over the linear range of the specific antibiotic assay. Ultimately, this information can be used to determine the mass balance after drug release so as not to incur some of the pitfalls of current antibiotic-releasing implants, sub-therapeutic drug dosing and development of antibiotic resistant pathogens.

While all samples demonstrated release profiles throughout the 8-week study duration, immediate differences were evident in the kinetics of release based on the type of antibiotic incorporated into the coating. Salt formulations of the antibiotics exhibited an initial bolus release of antibiotic, followed by a slow tapering of drug release at each time point; however, free-base (free-base) formulations of the drug show evidence of first order release kinetics (see FIGS. 19A, 20A, and 21A for ciprofloxin, rifampcin, and vancomycin, respectively).

Antimicrobial bioactivity. Bioactivity of each sample was determined using zone of inhibition assays (FIGS. 19B, 20B, and 21B for ciprofloxin, rifampcin, and vancomycin, respectively). Interestingly, the non-salt antibiotic formulations (ciprofloxacin and rifampicin) showed enhanced zones of inhibition with compared to the antibiotic salts (vancomycin and oxacillin). Alternatively, the addition of a water-non-solvent to the formulation had a very limited impact on the release as indicated by microbial killing. Microbial killing trends based on the diameter of the zone of inhibition for each antibiotic were comparable to the antibiotic release kinetic curves. Inhibition of microbial growth for non-salt antibiotic formulations also exhibited zero-order release kinetics out to 8 weeks.

The variation in antibiotic release kinetic curves (first-order or zero-order) based on the salt form of the antibiotic prompted an investigation of the antibiotic benefits of combination therapies. The antimicrobial bioactivity of two drugs in combination was accessed using ZOIs. Interestingly, no statistically significant changes were observed in these studies, suggesting that combinatory therapies, while not deemed synergistic or additive, would not be antagonistic.

Salt vs. Free-Base Formulation Comparisons.

The impact of antibiotic form (salt or non-salt) was assessed by determining the antibiotic release kinetic profiles and antimicrobial bioactivity using PCL/acetone coating formulations that contained either ciprofloxacin salt or ciprofloxacin free-base. Release profiles and bioactivity showed significant differences (see FIGS. 19A-21B). Table 3 shows the water solubility, formulation, thermostability, and of each antibiotics for reference.

TABLE 3

| | | Molecular | Water | pH induced | Solubility | Melting | | Anti- |
|---|---|---|---|---|---|---|---|---|
| | | weight | solubility | in the | in organic | temp. | Available | bacterial |
| Drug | Class | (g/mol) | (mg/ml) | surrounding | solvents | (° C.) | Forms | spectrum |
| Cipro- | Quino- | 331.4 | Insoluble | Amphoteric | High: MeOH | 255 (de- | Salt, Free- | Broad |
| floxacin | lones | | (0.001 | | (methanol), | composes) | base | spectrum |
| | | | mg/ml) | | DMF, DMSO, | | | |
| | | | | | Low: | | | |
| | | | | | Dioxane | | | |
| Oxacillin | Beta- | 401.4 | Slightly | High | High: MeOH, | 188 | Salt | Enteric |
| | lactam | | soluble | | DMSO, | | | bacteria |
| | | | (13.9 | | Low: EtOH | | | and other |
| | | | mg/ml) | | (ethanol), | | | eubacteria |
| | | | | | CHCl3, | | | |
| | | | | | methyl | | | |
| | | | | | acetate | | | |
| Rifampin | Rifa- | 823 | Slightly | Lipophilic, | DMSO, CHCl3, | 183-188 | Free-base | Gram- |
| | mycin | | soluble | low | ethyl | | | positive |
| | | | (1.4 | ionization | acetate | | | and |
| | | | mg/ml) | | methanol, | | | fastidious |
| | | | | | THF, | | | Gram- |
| | | | | | Low: | | | negative |
| | | | | | acetone | | | bacteria. |
| | | | | | | | | Myco- |
| | | | | | | | | bacteria |
| Tobra- | Amino- | 467.5 | Highly | Low | EtOH | 168 | Salt | Gram- |
| mycin | glycoside | | soluble | | | | | negative |
| | | | (538 | | | | | |
| | | | mg/ml) | | | | | |
| Vanco- | Glyco- | 1449.3 | Highly | Amphoteric | DMSO | 185-188 | Salt | Mainly |
| mycin | peptide | | soluble | | | | | Gram- |
| | | | (N100 | | | | | positive |
| | | | mg/ml) | | | | | bacteria. |
| | | | | | | | | Myco- |
| | | | | | | | | bacteria |

Thus, the formulation appears to affect the release kinetics of antibiotic from a polymer-membrane. Nosocomial osteomyelitis remains a significant clinical challenge associated with orthopedic surgeries due to a combination of bone's inherent avascularity, surgically compromised vascular supply, prevalent void spaces, and necrotic tissue. These factors not only limit the body's ability to combat opportunistic infections but also provide a favorable environment allowing invading microbes to evade the immune response, potentially coordinating the infection via biofilm development. The prevalent use of systemic antibiotics or antibiotic leaching orthopedic products has inadvertently promoted the development of antibiotic resistant microbes. Local antibiotic delivery offers a promising solution to combat these challenging opportunistic infections. During the course of this study, a polymer-coated allograft bone void filler was developed to release a variety of antibiotics locally, in a polymer controlled manner. The polymer coating and antibiotic constituents can be varied to provide either a bolus or sustained antibiotic release over the clinically relevant time frame of 8 weeks. More importantly, this technique has also allowed the development of a combinatorial approach to combating infection that may provide an important advance in reducing surgically introduced infections both acute and chronic following both primary and revision arthroplasty.

By utilizing a combinatory approach, i.e. the use of multiple drugs released from different fragments surgically implanted in a single void, surgeons can potentially minimize antimicrobial drug resistance, provide localized drug application, and provide protection against a broad spectrum of microbes. Over the past decade, drug resistance microbes have emerged as perhaps the greatest threat to surgical success (Peters et al., J Infect Dis 2008; 197: 1087-93). A major factor in drug resistance is leaching of drug at sub-minimal inhibitory concentrations for a sustained period of time. Combinatory approaches would enhance antibiotic protection by providing a more sustained drug release above the MIC while simultaneously decreasing the amount of either drug administered. Furthermore, the drug is localized to site of infection, allowing the host to maintain the native bacterial flora as well as minimize the nephro- and oto-toxic side effects common to many antibiotics during systemic administration.

Past studies utilizing tobramycin in this drug delivery system suggest that by mixing polymer and coating techniques this system can be engineered to not only minimize infections but also promote healing. A localized, sustained release out to 8 weeks provides a significant advancement in controlled drug release. In vivo assessment of this system will provide additional insight into the system's efficacy and practicality for clinical application.

### Example 3

### Bone Implants in Mouse

In this example, the generation 2 fabrication was tested in vivo in mice.

For these studies, C57 Black mice were used, with 3-5 animals per group. Morselized allograft bone was coated by solvent casting in a PCL tobramycin solution (PCL—60 mg/ml 10 kD; 10% tobramycin). This bone implant was a generation 2 fabrication made using the method schematically depicted in FIG. 7B). As a control, uncoated allograft particulate was used. [All implants were sterilized via ethanol prior to implantation. A small (approximately 1-2 cm) incision was made on the back of the mouse after the area was shaved and sterilized. A subcutaneous pocket was created by blunt dissection. The implant was placed in this subcutaneous pocket and the incision was sutured closed. A 10⁵ to 10⁷ injection of E. coli was placed into the same subcutaneous pocket as the implant (i.e., injected after the incision was sutured closed).

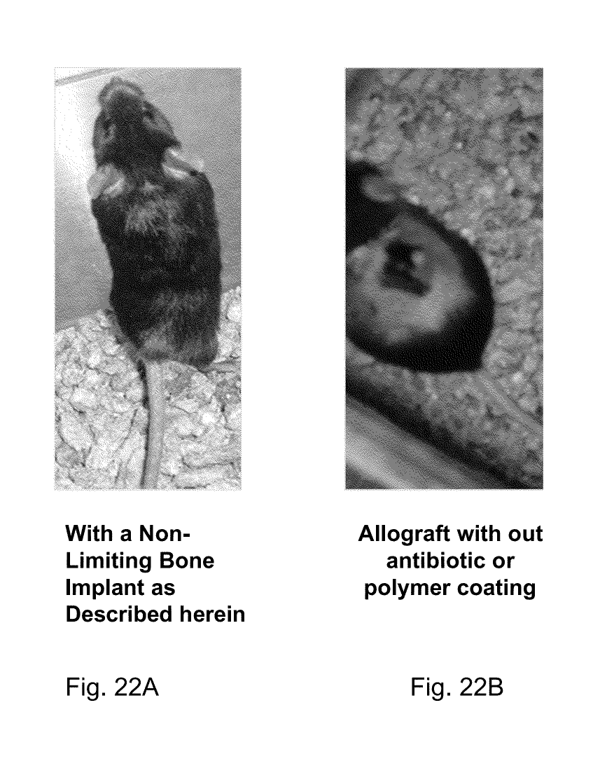

Mice were assessed post-implant for appearance and behavior using a modified Petty scale to assess these attributes (see Rao, N. et al., Plast. Reconstr. Surg. 127, Suppl. 1, 177S-187S, 2010). Representative individuals are shown in FIGS. 22A and 22B. The animal in FIG. 22A was implanted with a non-limiting bone implant fabricated using the generation 2 fabrication method by dip-coating morselized allograft bone in a polymer solution containing tobramycin. To make the implant shown in FIG. 22A, morselized bone was put into the polymer solution and mixed. The acetone solvent was allowed to evaporate leaving a wafer like consistency, which was subsequently sterilized with ethanol. The animal in FIG. 22B was implanted with morselized bone that was sterilized with ethanol. During sterilization, the morselized particulate was fused together in a wafer like consistency, and this “wafer” was implanted. As can be seen, the mice implanted with the tobramycin-coated generation 2 fabrication were able to heal better than mice implanted with a drug-free implant (compare FIG. 22A to FIG. 22B).

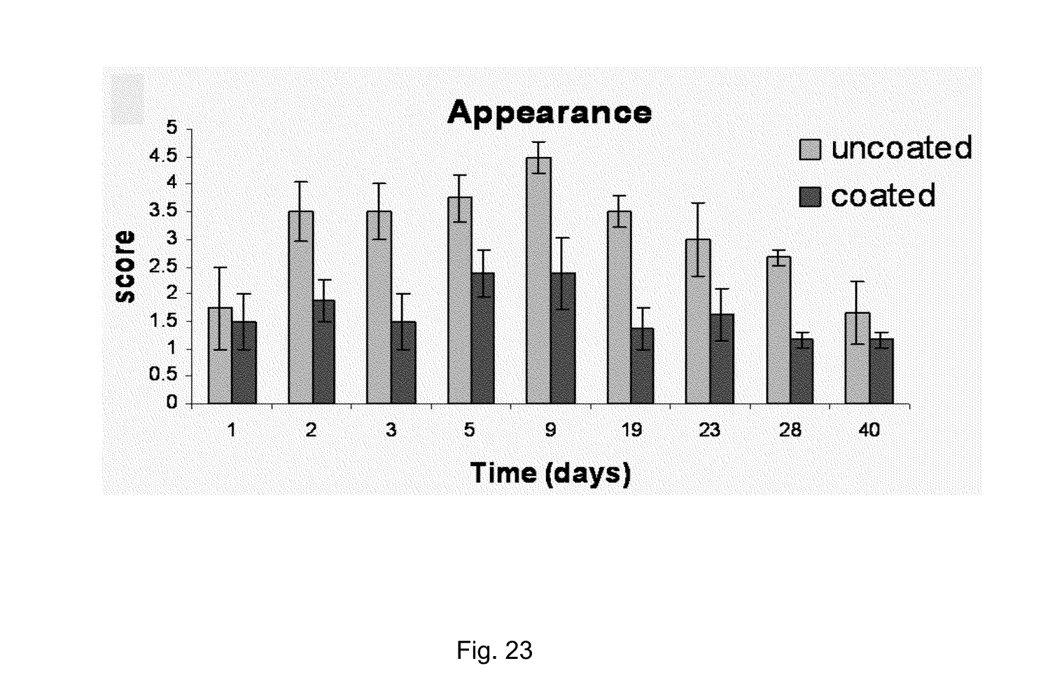

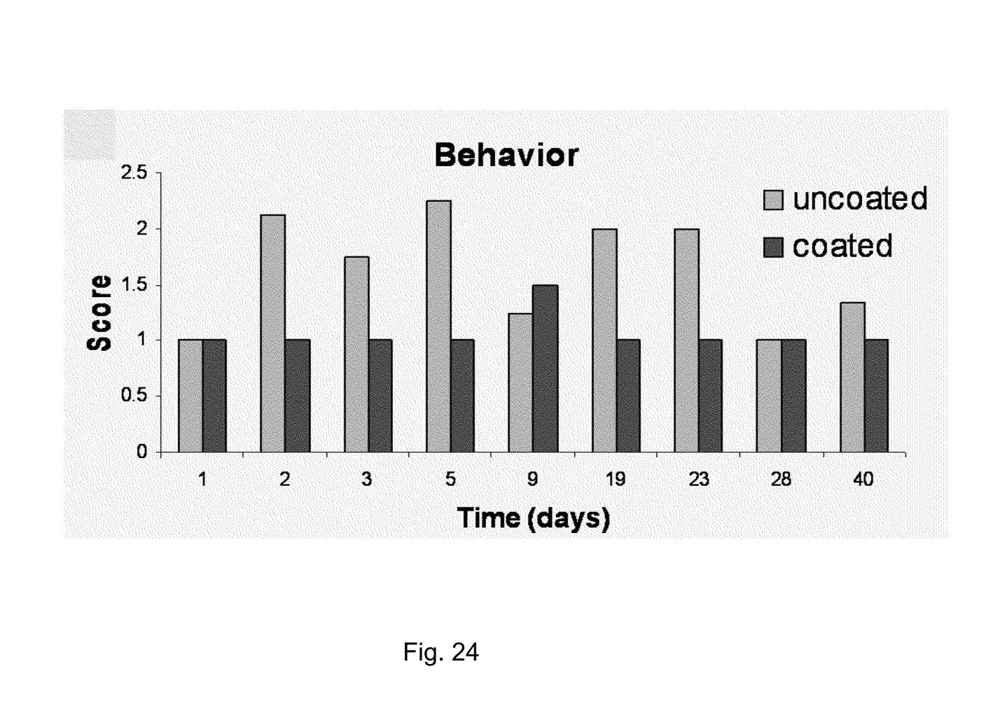

Additionally, using a modified Petty scale, the implanted mice (n=9 mic total) were assessed for appearance and behavior up to forty days (i.e., almost 6 weeks) post implant. In this assessment, the higher the score, the more “unnatural” the animal appeared and behaved. As shown in FIGS. 23 and 24, the tobramycin-coated implant receiving mice also appeared healthier (FIG. 23) and showed more alert behavior (FIG. 24) as compared to rice receiving a drug-free implant (red bars in FIGS. 23 and 24).

### Example 4

### Protocol for Making Implants Having a Uniform Mixture (Generation 3)

As described in the Examples above, addition of PEG to the implant helped with the phase separation problem and altered the kinetics of the drug release. However, PEG did not fully cure the phase separation problem. Thus, the generation 3 fabrication method was developed as a molten cast method. To generate approximately twenty solid implants having a uniform mixture (where each implant is 2 mm×2 mm×6 mm in size), the following protocol is used. This process is schematically depicted in FIG. 7C. Note that this size of implant (i.e., the 2 mm×2 mm×6 mm) was chosen because the intended host recipient of the generation 3 fabrication implant described in this example is a rabbit. For larger vertebrate host subjects (e.g., a human), obviously a larger implant would be prepared, but the protocol would be the same, just with more ingredients and a larger mold.

Additionally, although the below protocol uses the thermostable drug tobramycin, any other thermostable drug may be used. If the drug is an antibiotic, such non-limiting thermostable antibiotics include tobramycin, gentamicin, vancomycin, a cephalosporin, or a mixture of two or more of tobramycin, gentamicin, vancomycin, and a cephalosporin

For this protocol, the materials used were:

ProOsteon 500R (commercially available from Biomet, Inc., Warsaw, Ind.)

PCL 10 KD (commercially available from Sigma-Aldrich Co, St. Louis, Mo., catalog #: 440752)

PEG 20 KD (commercially available from Sigma-Aldrich Co, St. Louis, Mo., catalog #: P2263)

Acetone

Tobramycin (commercially available from Research Products International Corp., Mr. Prospect, Ill., catalog #: T45000-1.0) or Microencapsulated Tobramycin (Maxx Performance, Inc., Chester, N.Y.) or any other thermostable drug of interest

Other materials included weigh boats, Glass Petri Dishes, Spatulas (2×), Round Bottom Flask with a stir bar, Slide Molds, Silicone Isolators, Mortar and Pestle, water bath. −20° C. freezer, hot plate, and an external temperature probe.

To generate the implants, the following steps were taken: First, the Water Bath was heated to 45° C. and the hot plate is heated to 80° C. Next, the amount of polymer/drug/bone void filler needed was calculated. For example, if the bone component was about 64% ground, then 0.7 grams of morselized ProOsteon was used, 0.3 grams of PEG/PCL combination (all ratios by weight) was used, and 0.1 grams of Tobramycin was used. Next the BoneVoid Filler ProOsteon was morselized with mortar and pestle. The quality of morselization of the ProOsteon was evaluated under a dissecting microscope to ensure consistency of the particles. The morselized ProOsteon, Polymer mixture (i.e., PCL and PEG), and Tobramycin were then weighed out according to calculations determined above (i.e., 0.7 grams morselized ProOsteon, 0.3 grams PEG/PCL combination, and 0.1 grams of tobramycin). Note that the ratio of PCL and PEG was changed according to the desired degradation properties, but typically varies between 75% PCL and 25% PEG and 90% PCL and 10% PEG in relation to the polymer component of the formulation (all ratios by weight) So, for example, if 1 gram of total mixture was desired with 90% PCL and 10% PEG, then the final mixture (i.e., that was poured into the mold) contained 700 mg of bone, 270 mg of PCL, and 30 mg of PEG.

As depicted schematically in FIG. 7C, in the generation 3 fabrication method, allograft or synthetic bone substitute was ground with a mortar and pestle and the polymers (PCL and PEG) where melted over heat. Once the polymers were melted, granulated bone was added (greater than 65% w/w) along with tobramycin. This molten mixture was then compressing into a silicone mold. After the mixture solidified, it was removed from the mold. The advantage of this generation 3 process is that the bone implants were be produced in customized geometries with precise amounts of drug, polymer, and bone mixed uniformly throughout the implant

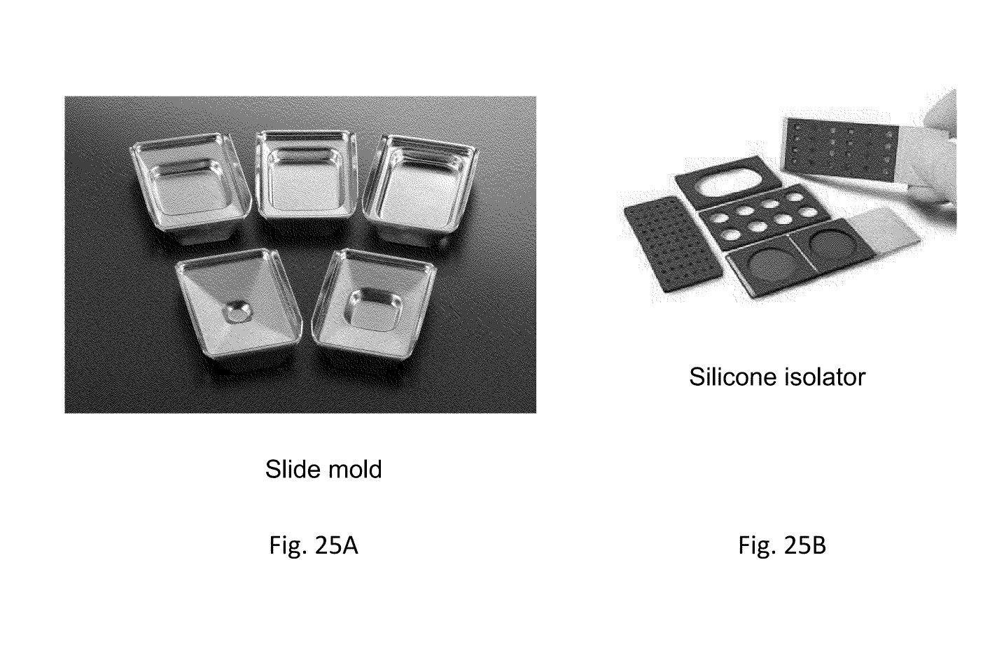

To generate the implants, the polymer (i.e., the mixture of PEG and PCL) was put into the slide mold (e.g., as depicted in FIG. 25A) on the 80° C. heat plate for approximately 15 to 30 minutes, until the polymer was consistently melted. The polymer mix was stirred with spatula at approximately 5 minute time intervals.

As an optional step, while waiting for the polymer to heat for 15-30 minutes at 80° C., a PCL solution of 60 mg/mL in acetone was prepared. To do this, 10 kD PCL is added to acetone at a concentration of 60 mg/ml using the 45° C. water bath with stirring.

Next, morselized ProOsteon and tobramycin were added to the melted polymer mixture. The resulting polymer/ProOsteon/tobramycin mixture was mixed well, especially in the corners of the slide mold.

Next, the silicone isolator (e.g., such as one depicted in FIG. 25B) was adhered to a piece of foil or the bottom of a plastic petri dish and placed on the hot plate. In some embodiments, to prevent the implant from hardening too rapidly, the silicone mold can be packed while warm.

Using the spatulas, each space in the mold with the silicone isolator was filled with the polymer/ProOsteon/drug molten mixture and compress. Excess polymer was scraped away before it solidified.

After silicone isolator was filled, the mold is placed in the freezer (−20° C.) for at least 5 minutes.

Then, the mold was removed from freezer and excess polymer/ProOsteon/drug was scraped away from the silicone isolators.

The isolator was then peeled off the foil and the implants pushed out.

If the PCL solution of 60 mg/ml in acetone was made earlier, as an optional step, each implant may be dipped in this solution. This optional step may create a “sealing” coat. In other words, in this embodiment, the resulting implant with the uniform mixture of polymer/ProOsteon/drug is additionally coated with a PCL coat. To do this, each implant was dipped in the PCL/acetone solution for about 30 seconds, and then allowed to dry for approximately 2 minutes. The implant is turned over and dipped again for about 30 seconds, and then allowed to dry for about 2 minutes. The dipping/drying process was repeated three or more times.

Each implant was next tested for quality assurance.

To do this, the bone void filler was weighted (+/−5%). Generally, a 2 mm by 2 mm by 6 mm implant had a weight of 37.5+/−1.875 (5%) milligrams.

Also, the length, width, and height were measured (+/−5%). A 2 mm by 2 mm by 6 mm length should be +/−0.05 mm for width and height and 0.15 mm for length.

Also, under a dissecting microscope, smoothness is looked for. In some embodiments, the resulting implant does not have major voids. Similarly, under a dissecting microscope squareness is looked for. In some embodiments, the resulting implant has crisp 90° angles.

From every batch, an SEM (scanning electron microscopy) was taken of one implant to ensure consistency of blended material (i.e., to ensure a uniform mixture in the solid implant)

Also, from every batch, an SEM was taken of one implant after 1 day release to ensure porosity of sample. Note sample porosity is created when the PEG dissolves from the implant.

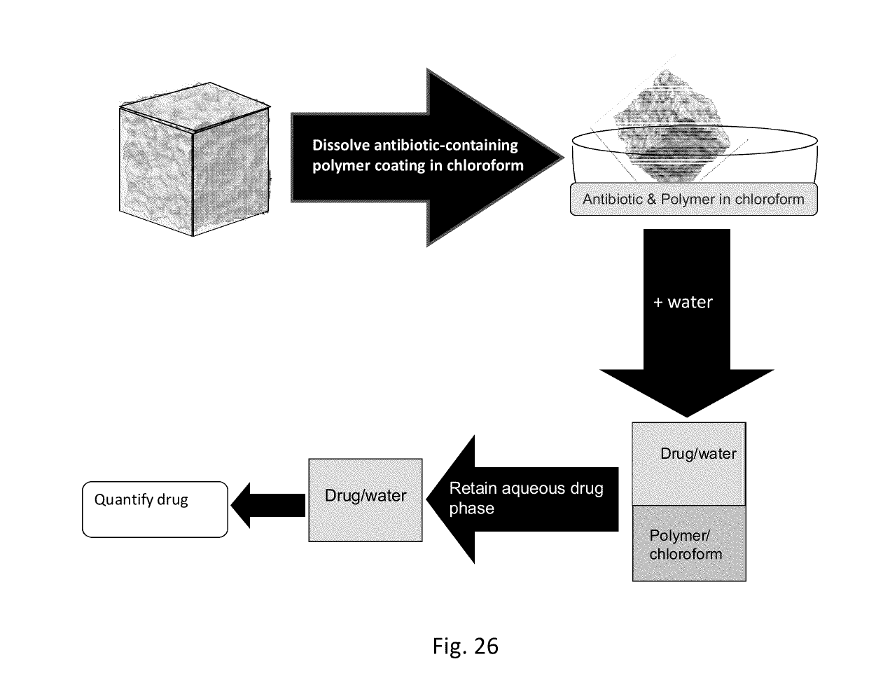

Compression and cyclic compression mechanical tests by applying pressure to each of the dimensions of the sample using an Instron testing system with BlueHill software is also performed on at least one implant of every batch to determine isotropy. It should be noted that the actual amount of antibiotic in each bone implant made using any method described herein (including, without limitation, the method described in this Example 3) can be detected by standard methods. For example, as shown in FIG. 26, the antibiotic-containing polymer coating can be completely dissolved in chloroform, with that resulting solution mixed with water. A bilayer solution of water: chloroform will result, with the antibiotic present in the water layer and the polymer present in the chloroform layer. The water layer (i.e., the aqueous layer of FIG. 26) can be then retained (e.g., drawn off with a pipette) and the amount of antibiotic drug present quantitated using standard methods (e.g., HPLC). Using this method, the quantity of antibiotic drug actually deposited onto the allograft bone samples during the coating process can be determined. By similar methods, the quantity of antibiotic drug contained in any of the various bone implants describe herein (e.g., the bone implants generated using the methods schematically depicted in FIGS. 7A-7C) can be determined by dissolving the entire crouton in an organic solvent (e.g., phenol and/or chloroform).

### Example 5

In this example, the generation 3 fabrication generated according to the methods described in Example 4 is used in vivo in rabbits.

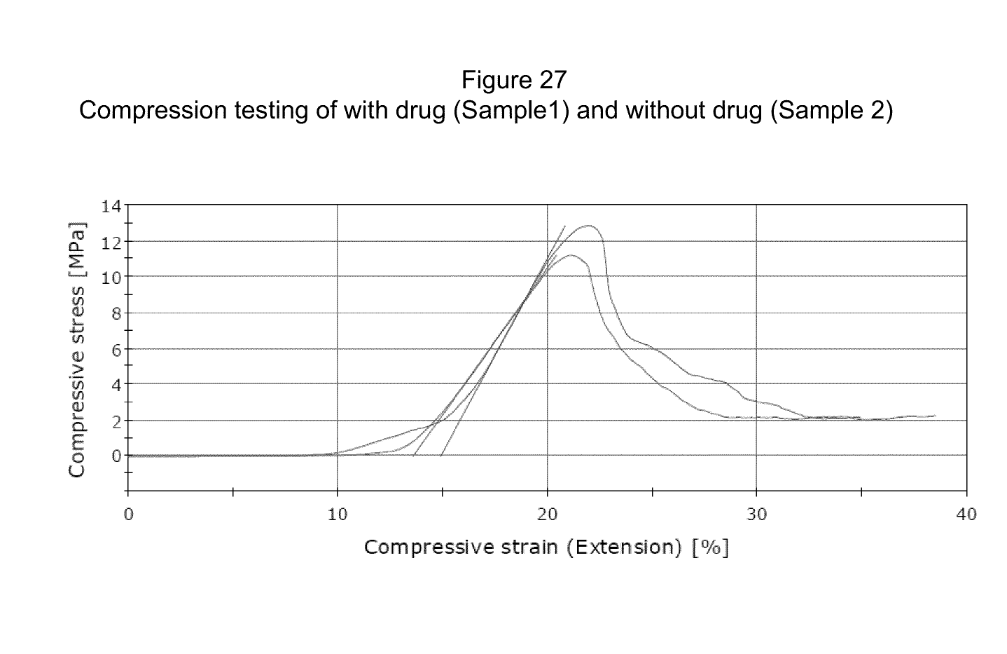

First, a compression test was performed to look at the strength of the bone implants with or without drug. A 1% compression is typical in bone cyclical tests because it approximates the amount of strain during walking. The results of these studies are shown in FIG. 27 and in Table 4 below.

TABLE 4
(Compression Strain)

| | | Compressive | Modulus (Young's |
|---|---|---|---|
| | Maximum | Strength | Compressive Stress |
| | Load [N] | [MPa] | 28 mm-10 mm) [MPa] |
| 1 | 151.37 | 11.21 | 163.92551 |
| 2 | 173.42 | 12.85 | 217.11218 |
| Mean | 162.39 | 12.03 | 190.51885 |
| Standard | 15.59025 | 1.15483 | 37.60866 |
| Deviation | | | |
| Minimum | 151.37 | 11.21 | 163.92551 |
| Maximum | 173.42 | 12.85 | 217.11218 |
| Range | 22.05 | 1.63 | 53.18667 |

As shown in FIG. 27 and in Table 4, there is a slight difference in the strength of generation 3 fabrications with and without drug. Almost 90 different generation 3 fabrications were tested.

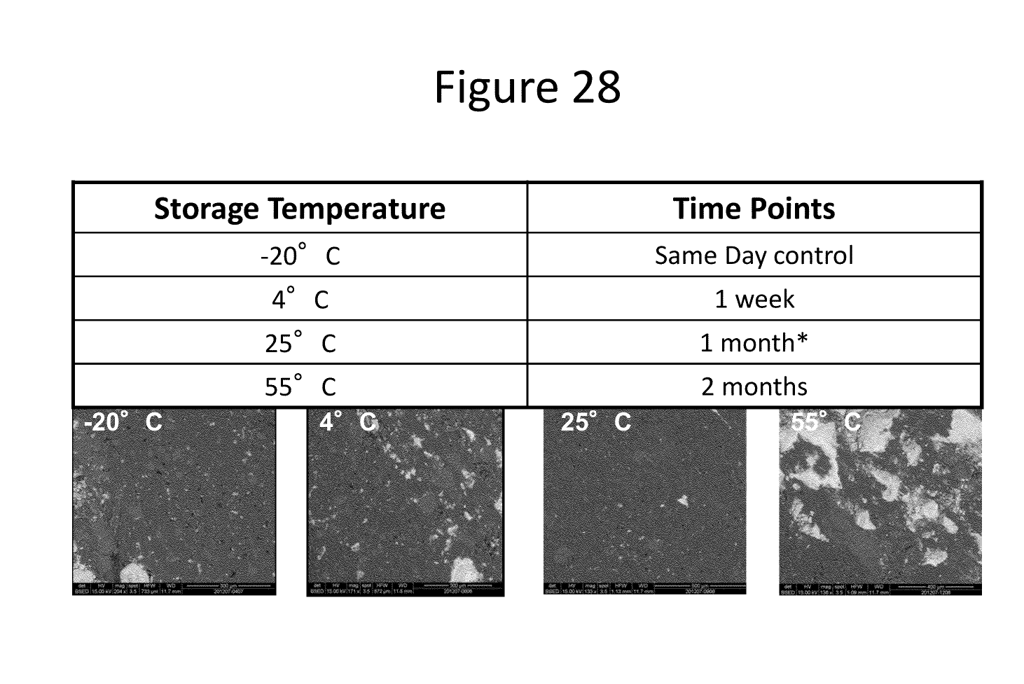

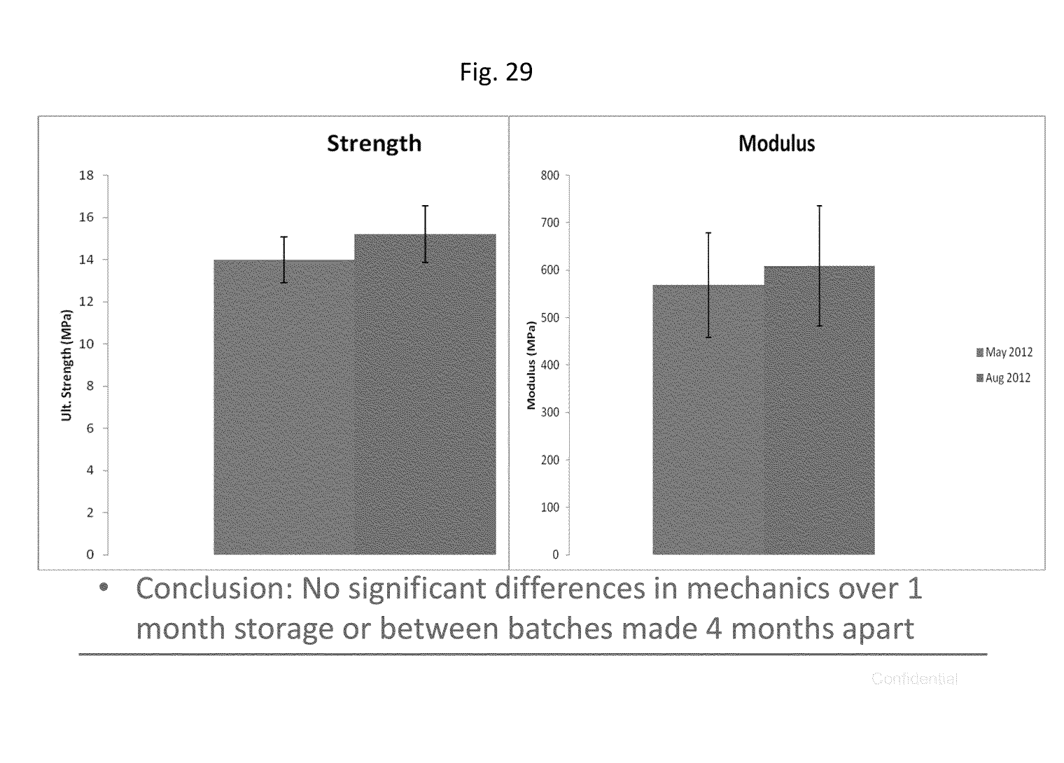

The shelf-life of the generation 3 fabrication was next confirmed. The generation 3 fabrications were stored at four different temperatures (i.e., −20° C., 4° C., 25° C., and 55° C.), and then at specific time points, a designated number of implants were removed from storage and subjected to scanning electron microscope (SEM) imaging and mechanical testing. FIG. 28 shows SEM images of an implant stored at −20° C. on the same day (left), an implant stored at 4° C. 1 week (second from left), an implant stored at 25° C. for 1 month, and an implant stored at 55° C. for two months (right). Storage at 55° C. for 60 days was found of be equivalent to storage for 1 year at 25° C. However, there were no detectable differences in bioactivity (data not shown). Similarly, there is no significant different in the strength of modulus of bone implants regardless of their manufacturing date or storage conditions (see FIG. 29).

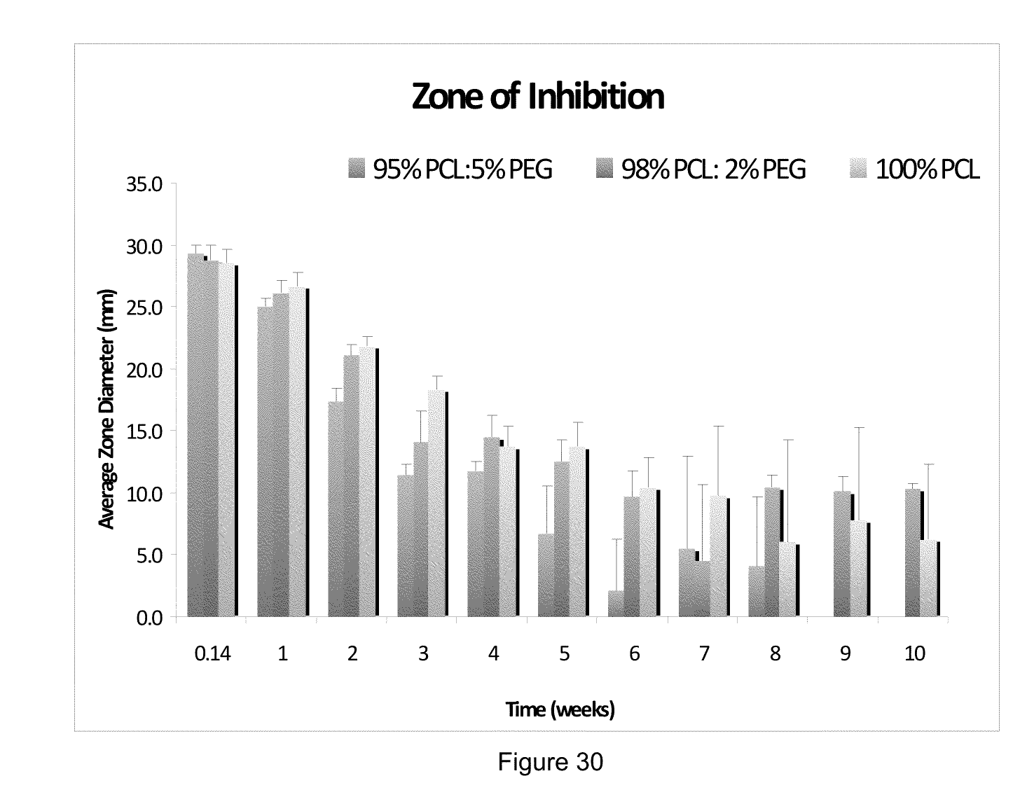

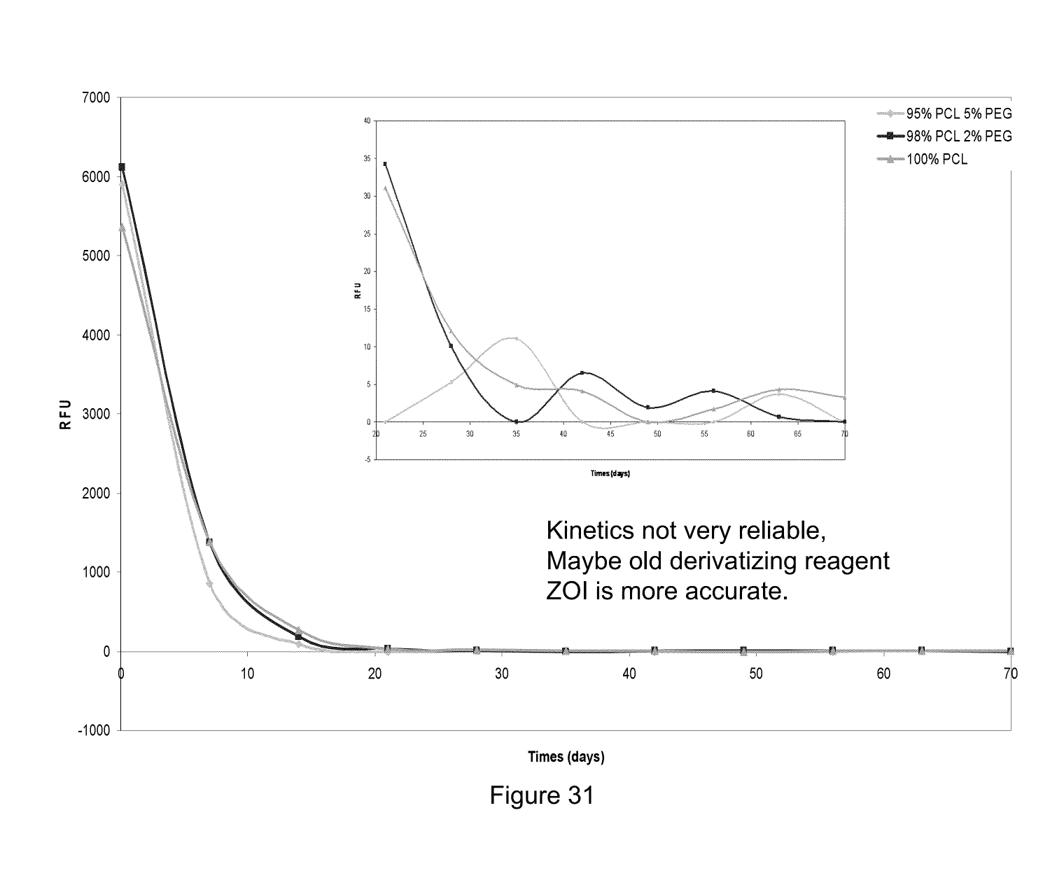

To confirm bioactivity of generation 3 fabrications based on a difference in the ratio of PCL to PEG, ZOI data was collected. The fabrications tested were 95% PCL: 5% PEG ratio by weight in the polymer portion of the fabrication which is 27% of the total formulation (the other components being 10% drug and 63% bone) (blue bars in FIG. 30), 98% PCT: 2% PEG ratio by weight in the polymer portion of the fabrication which is 27% of the total formulation (the other components being 10% drug and 63% bone) (red bars in FIG. 30), and 100% PCL ratio by weight in the polymer portion of the fabrication which is 27% of the total formulation (the other components being 10% drug and 63% bone) (green bars in FIG. 30). Generally, FIG. 30 shows that an increase in the PEG component is related to an increase in the rate of tobramycin release, based on the bioactivity. As can also be seen in FIG. 30, 98% PCL: 2% PEG still had strong killing of bacteria at 10 weeks. FIG. 31 shows the kinetics that correspond to the ZOI data of FIG. 30. As can be seen in FIG. 31, kinetics data are not as reliable as the ZOI data (FIG. 30).

Next, an experiment was performed to determine if bacteria could still grow in the presence of liquid released from a generation 3 fabrication bone implant. It was thought that if bacteria didn't grow in the liquid media, but a sample of that media grows on solid agar nutrients, the bacteria may have still been alive, meaning the drug was not bacteriacidal (i.e., killing bacteria) but merely stopped bacteria from growing (bacteriostatic). For these studies, each generation 3 implant was placed in phosphate buffered saline (PBS) and the PBS collected at different time points. The PBS was then added to a bacterial culture and the growth of the culture was determined using absorbance at. Subsequently, bacteria from the culture were plated on blood agar plates and the resulting colonies counted.

The results showed that molecules leaching from the polymer coating of the generation 3 fabrication bone implant seemed to have a bacteriostatic effect. Moreover, if a dosage of at least 10⁷ CFU (colony forming unit) were added, the bacteria could grow. The implication is that if the generation 3 fabrication can block active bacteria growth (i.e., hold the bacteria to at least 10⁵ CFU, then the rabbits' immune systems can overcome the infection. The results of this study are shown in Table 5.

TABLE 5
Bacteria Growth in the Presence of Release Liquid

| Sample | Anti- | Liquid | Agar | | Time | |
|---|---|---|---|---|---|---|
| # | biotic | Growth | Growth | Inoculum | point | |
| 5 | Yes | No | No | 10\hat{ }5 | 24 | hours |
| | | | | (i.e., 10⁵) | | |
| 5 | Yes | No | NA | 10\hat{ }7 | 24 | hours |
| 5 | Yes | No | NA | 10\hat{ }5 | 4 | days |
| 5 | Yes | No | NA | 10\hat{ }7 | 4 | days |
| 5 | Yes | No | No | 10\hat{ }5 | 24 | hours |
| 5 | Yes | No | No | 10\hat{ }7 | 24 | hours |
| 5 | Yes | No | No | 10\hat{ }9 | 24 | hours |
| 5 | Yes | No | No | 10\hat{ }5 | 4 | days |
| 5 | Yes | No | No | 10\hat{ }7 | 4 | days |
| 5 | Yes | No | No | 10\hat{ }9 | 4 | days |
| 5 | No | No | Confluent | 10\hat{ }5 | 24 | hours |
| 5 | No | Yes - | NA | 10\hat{ }7 | 24 | hours |
| | | Not visual | | | | |
| 5 | No | Yes | NA | 10\hat{ }5 | 4 | days |
| 5 | No | Yes | NA | 10\hat{ }7 | 4 | days |
| 5 | No | No | 2 colonies | 10\hat{ }5 | 24 | hours |
| 5 | No | No | 10 colonies | 10\hat{ }7 | 24 | hours |
| 5 | No | NA | Confluent | 10\hat{ }9 | 24 | hours |
| 5 | No | Yes | Confluent | 10\hat{ }5 | 4 | days |
| 5 | No | Yes | Confluent | 10\hat{ }7 | 4 | days |
| 5 | No | NA | Confluent | 10\hat{ }9 | 4 | days |

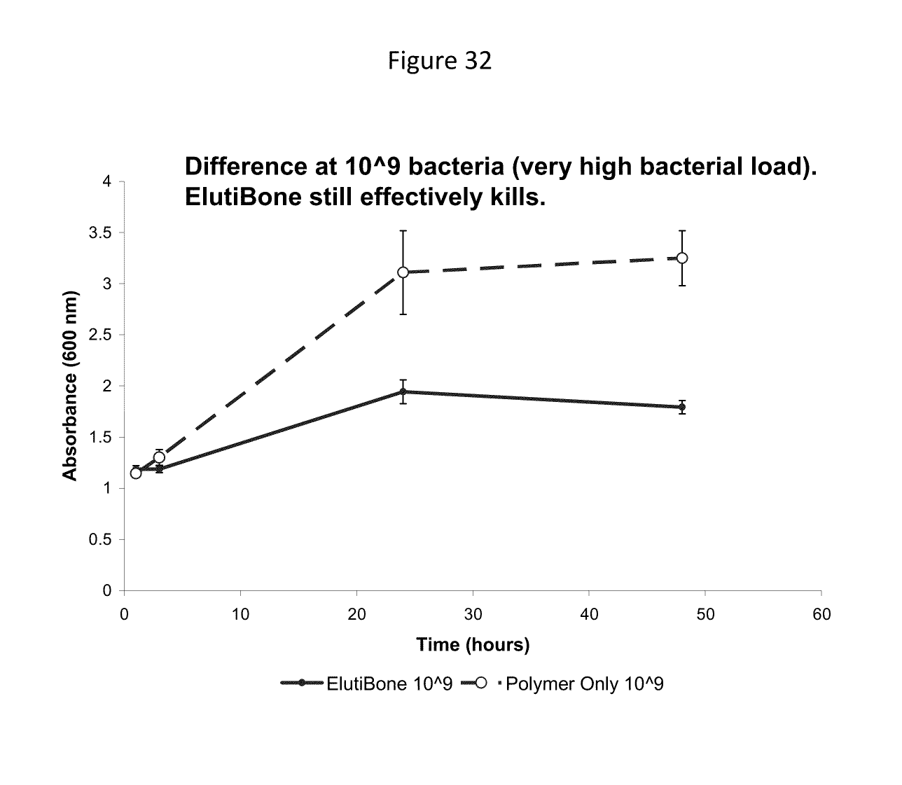

Interestingly, the polymer itself was found to have bacteriostatic effects, but those effects could be overcome by using a higher initial amount of bacteria. As shown in FIG. 32, at a difference of 10⁹ bacteria (a very high bacterial load), the drug-containing generation 3 fabrication (solid line) was still able to effectively kill as compared to an implant coated with polymer only and no drug (dotted line).

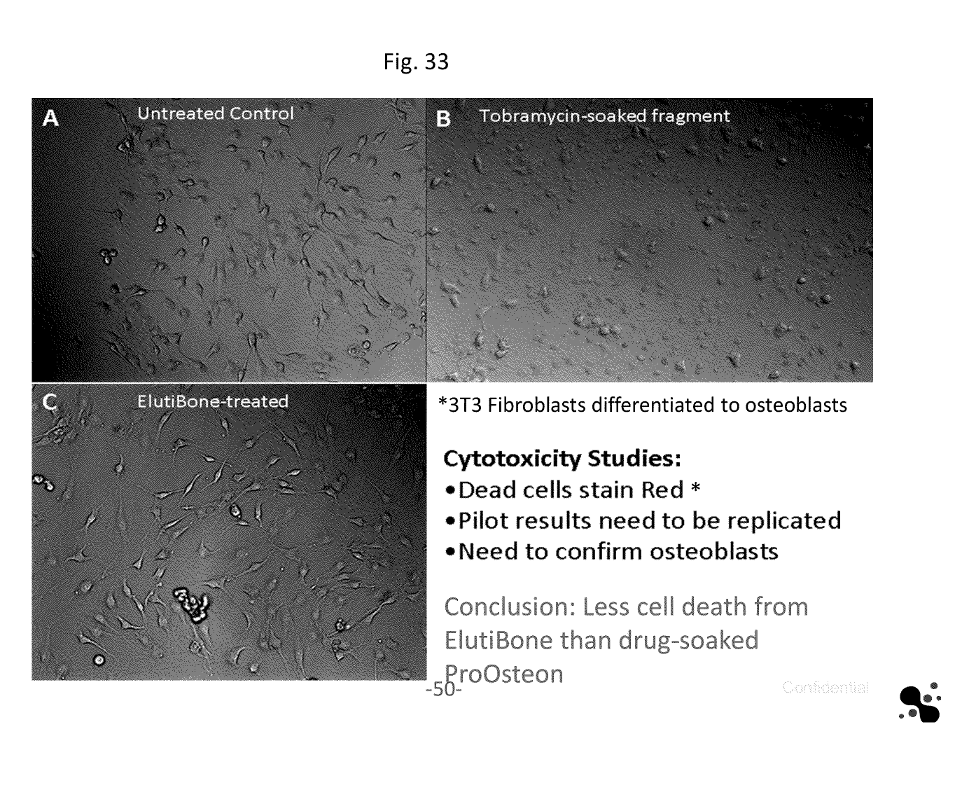

The effect of the generation 3 fabrication on osteoblasts was next tested. For these studies, osteoblasts grown in vitro were placed in the presence of the generation 3 fabrication itself, tobramycin-soaked generation 3 fabrication, or untreated control ProOsteon. As shown in FIGS. 33A-33C, drugless generation 3 fabrication did not kill cells (FIG. 33C); however the tobramycin-soaked generation 3 fabrication did kill cells (dead cells stain red).

Additional references for this work include:

1) Fátima Varanda et al, Solubility of Antibiotics in Different Solvents. 1. Hydrochloride Forms of Tetracycline, Moxifloxacin, and Ciprofloxacin, Jul. 29, 2006

2) For solubilities: http://www.pharmacopeia.cn/usp.asp

3) Woodruff, Maria A. and Hutmacher, Dietmar W. (2010) The return of a forgotten polymer: Polycaprolactone in the 21st century. Progress in Polymer Science

### Example 6

### Bone Implants in Rabbit and Sheep In Vivo Models

For the in vivo studies, nine male, 3-4 kg New Zealand White rabbits (between 1-2 years of age) were used.

In the rabbit experiment, 9 rabbits were implanted with either the non-limiting generation 3 bone implant described herein that was not coated or the non-limiting bone implant described herein that was coated with an antibiotic-containing polymer coating. All implants were sterilized with ethylene oxide (EtO) prior to implantation

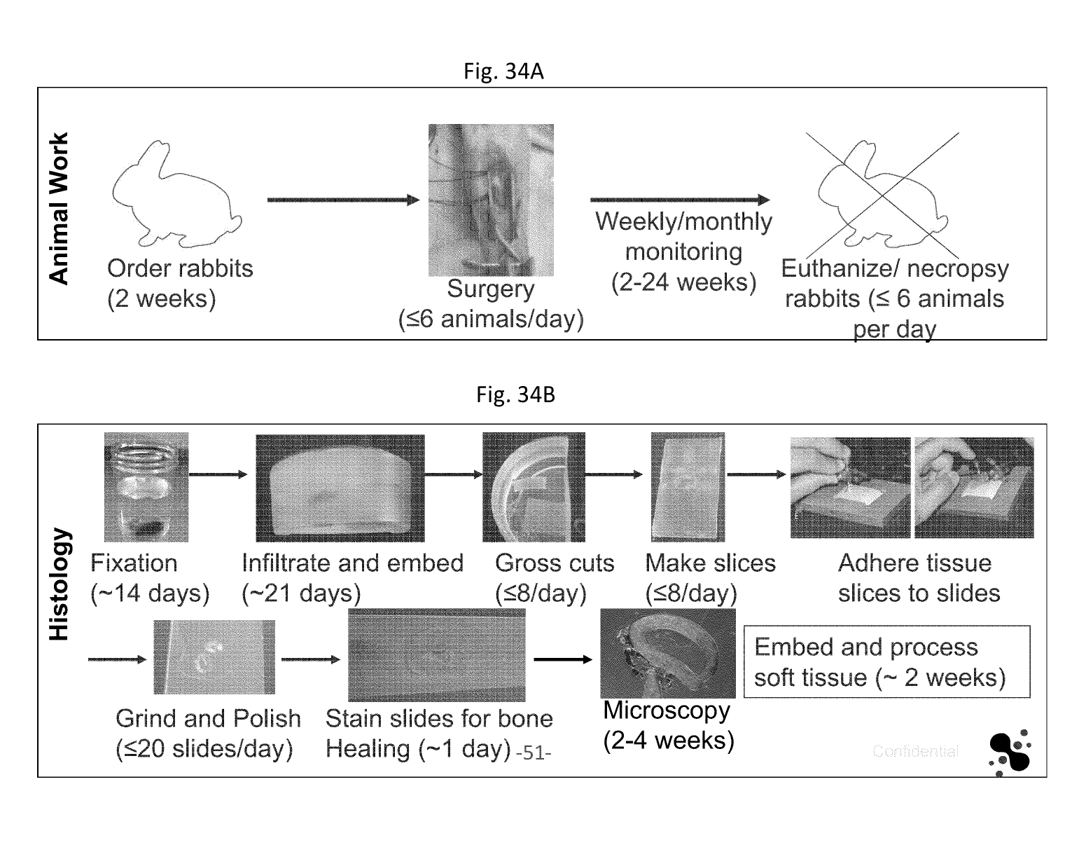

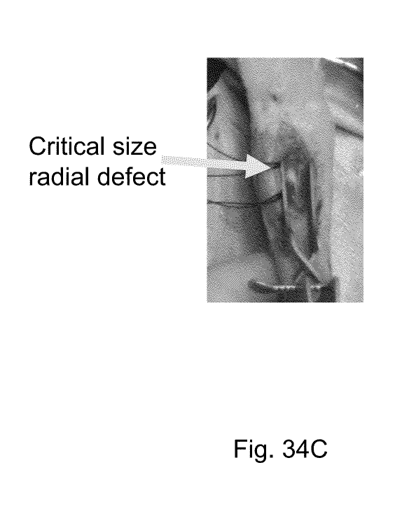

These timeline of these studies is schematically set forth in FIGS. 34A-34B in terms of animal work (FIG. 34A) and histology work (FIG. 34B).

For these studies, prior to surgery, the right forelimb of the rabbit leg was clipped, as was a small patch at the back of the head on the back, and 25 mcg Durageic (fentanyl) patch was placed on the back area. The surgical procedure was adapted from Smelter's and Koort's protocols (see Koort et al., Antimicrob Agents Chemother, vol. 49, pp. 1502-8, 2005; Koort et al., J Biomed Mater Res A, vol. 78, pp. 532-40, 2006; Smeltzer et al., J Orthop Res, vol. 15, pp. 414-21, 1997). Briefly, the radius of the right forelimb was exposed surgically and prepared by scrubbing with Povidine iodine and ethanol solution. A bone segment (approx. 6 mm by 2.7 mm by 2 mm) was drilled under saline cooling into the proximal medial metaphysis of the right tibia. Subsequently, 10⁵ to 10⁷ Colony Forming Unites (CFUs) of S. aureus (for the rabbits) were injected directly into the medullary canal anterior to the surgical site. The bone segment was then filled with a non-limiting bone implant containing 90% PCL:10% PEG prepared as described above in Example 5, or with a suitable control bone graft replacement (approx. 6 mm by 2 mm by 2 mm). The wound was closed with resorbable sutures and the rabbit removed from the anesthetic and observed until it was awake and mobile. The animals were dosed with 100 mg of Cefazolin SQ injection 30 minutes post-surgery. Postoperative analgesics (Duragesic/Fentanyl) were administered to all animals immediately after surgery and transdermally for 2 days at a concentration of 25 mcg/hr. Analgesics were continued with any animal that avoided use of the affected forelimb. Pain levels (e.g., lack of appetite, shivering, and postural changes) were monitored for a minimum of 3 days postoperatively. The temperature and weight of each animal were monitored weekly as well as radiographic images of the surgical site. Note that the animal was terminated if the following conditions occurred: blood borne infection, overwhelming local infection, excessive signs of distress, appetite suppression as indicated by loss of weight, limited water consumption, and/or lethargy.

For these studies in rabbits, endpoint analyses included: (i) imaging of bone by X-Ray; (ii) microbiological culture of bone site and soft tissue surrounding surgical site; (iii) SEM and histological analyses of bone growth and (iv) high pressure liquid chromatography (HPLC) quantification of antibiotic excreted in the urine or still remaining in bone replacement at the conclusion of the study or termination of the animal.

The different cohorts of animals used in this study were as follows:

Cohort 1: No polymer, no drug, no infection. The implant used in this cohort 1 was a fragment of ProOsteon that was sculpted with a razor blade to be 2 mm×2 mm×6 mm in dimensions. There was no polymer and no drug used to fabricate the implant, and no infection was introduced into the surgical site. This cohort 1 was used as a control that allowed an assessment of the surgical technique and the sterility conditions.

Cohort 2: No polymer, no drug, 10⁵ CFU S. aureus. The implant used in this cohort 2 was a fragment of ProOsteon that was sculpted with a razor blade to be 2 mm×2 mm×6 mm in dimensions. There is no polymer and no drug used to fabricate the implant. 10⁵ CFU of S. aureus was introduced into the medullary canal anterior to the surgical site on the tibia. This cohort 2 was a control that allowed an assessment of the surgical technique and the sterility conditions.

Cohort 3: PCL-PEG coat, no drug, no infection. The implant used in this cohort 3 was morselized ProOsteon that was mixed using the generation 3 fabrication method in a ratio of 70% ProOsteon and 30% polymer. The polymer was melted at 75° C. in a ratio of 90% PCL and 10% PEG. The mixture was then packed into the silicone isolator (dimensions of 2 mm×2 mm×6 mm). A final dip of each fabricated crouton into a PCL acetone solution (10 kD PCL at 60 mg/ml in acetone) was done prior to sterilization. For this cohort 3, there was no drug used in fabricating the implant, and no infection introduced into the surgical site (i.e., the implantation site). This cohort 3 was a control that allowed an assessment of the safety of the polymer components of the generation 3 fabrication. Note that no data is shown from this control cohort 3 as it was unremarkable and looked like the results from cohort 1.

Cohort 4: PCL-PEG coat, no drug, 10⁵ CFU S. aureus. The implant used in this cohort 4 was morselized ProOsteon that was mixed using the generation 3 fabrication method in a ratio of 70% ProOsteon and 30% polymer. The polymer was melted at 75° C. in a ratio of 90% PCL and 10% PEG. The mixture was then packed into the silicone isolator (dimensions of 2 mm×2 mm×6 mm). There was no drug used to make the implant, but 10⁵CFU of S. aureus was injected into the medullary canal anterior to the surgical site. This cohort 4 is a control that allows an assessment of the impact of the polymer on the progression of the infection.

Cohort 5: No polymer, 10% drug soak, 10⁵ CFU S. aureus. The implant used in this cohort 5 was a fragment of ProOsteon that was sculpted with a razor blade to be 2 mm×2 mm×6 mm in dimensions. There is no polymer in the implant, but the implant was soaked in a 10% solution of tobramycin in water for 10 minutes prior to implantation and 10⁵ CFU of S. aureus was introduced into the medullary canal anterior to the surgical site on the tibia. This cohort 5 was a control that mimics what is currently being done in many human surgeries.

Cohort 6: PCL-PEG coat, 10% drug load, no infection. The implant used in this cohort 6 was morselized ProOsteon that was mixed using the generation 3 fabrication method in a ratio of 63% ProOsteon and 27% polymer. The polymer was melted at 75° C. in a ratio of 90% PCL and 10% PEG. 10% powdered tobramycin drug was added to this molten mixture of polymer and ProOsteon. The mixture was then packed into the silicone isolator (dimensions of 2 mm×2 mm×6 mm). A final dip of each fabricated crouton into a PCL acetone solution (10 kD PCL at 60 mg/ml in acetone) was done prior to sterilization. No infection was introduced into the surgical site. This cohort 6 was a control that allowed observation of how host bone reacted to the generation formulation for safety purposes.

Cohort 7: PCL-PEG coat, 10% drug load, 10⁵ CFU S. aureus. The implant used in this cohort 7 was morselized ProOsteon that was mixed using the generation 3 fabrication method in a ratio of 63% ProOsteon and 27% polymer. The polymer was melted at 75° C. in a ratio of 90% PCL and 10% PEG. 10% powdered tobramycin drug was added to this molten mixture of polymer and ProOsteon. The mixture was then packed into the silicone isolator (dimensions of 2 mm×2 mm×6 mm). A final dip of each fabricated crouton into a PCL acetone solution (10 kD PCL at 60 mg/ml in acetone) was done prior to sterilization. 10⁵ CFU of S. aureus introduced into the medullary canal anterior to the surgical implantation site on the tibia.

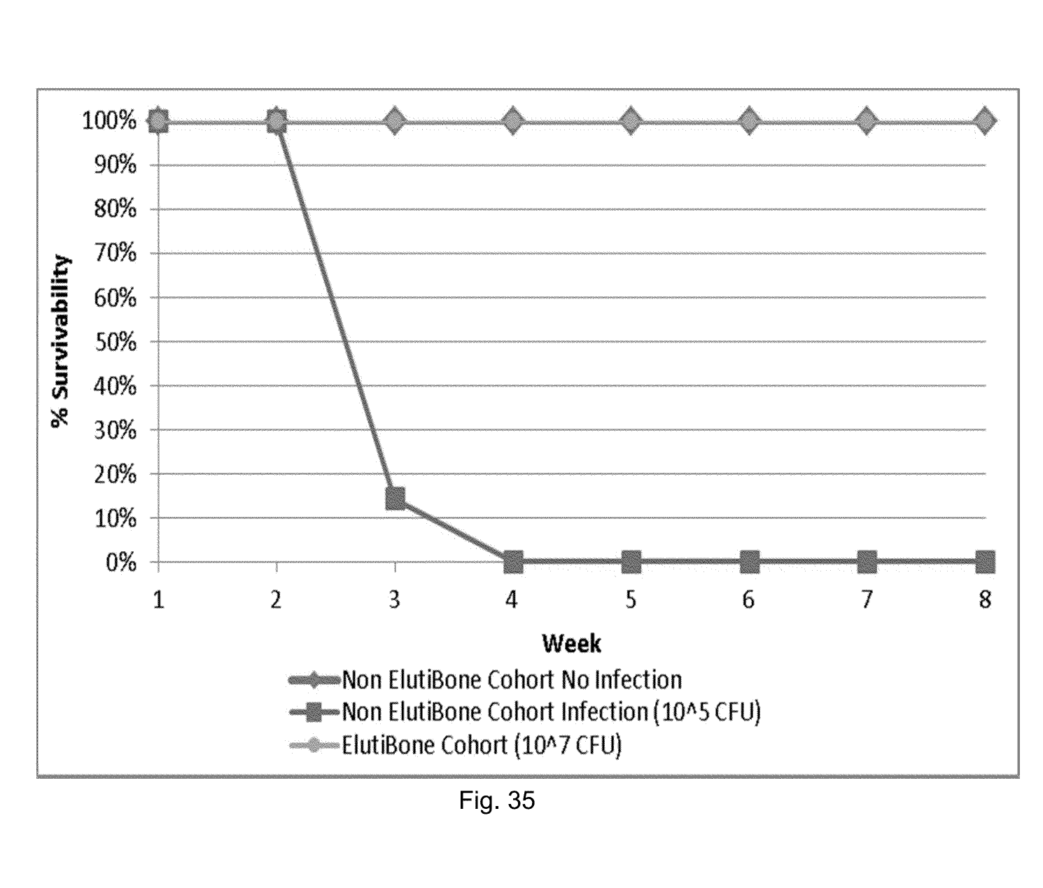

The surgeries were successful with no systemic infection seen in any of the nine animals. The implanted grafts provided mechanical stability. FIG. 34C (taken from FIG. 34A) shows a photograph from a representative individual, noting the critical size of the radial defect. FIG. 35 shows that the coated implant (green circles on FIG. 35) more than doubled the survivability of infected host animal implanted with the generation 3 fabrication as compared to infected animals implanted with a prior art implant.

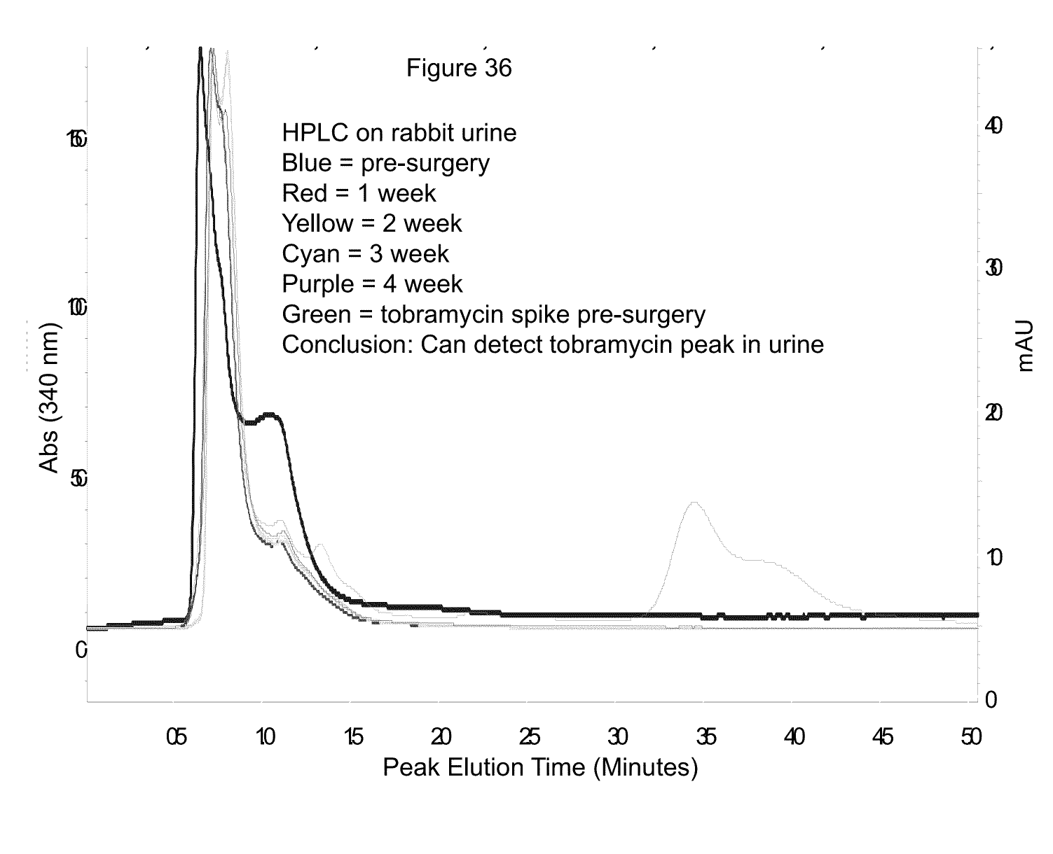

Using a high performance liquid chromatography (HPLC) protocol described above, the presence of tobramycin in rabbit urine was able to be traced following implantation of the tobramycin-soaked generation 3 fabrication. The data in FIG. 36 shows that the local release of tobramycin from the generation 3 fabrication did not affect the rabbit systemically.

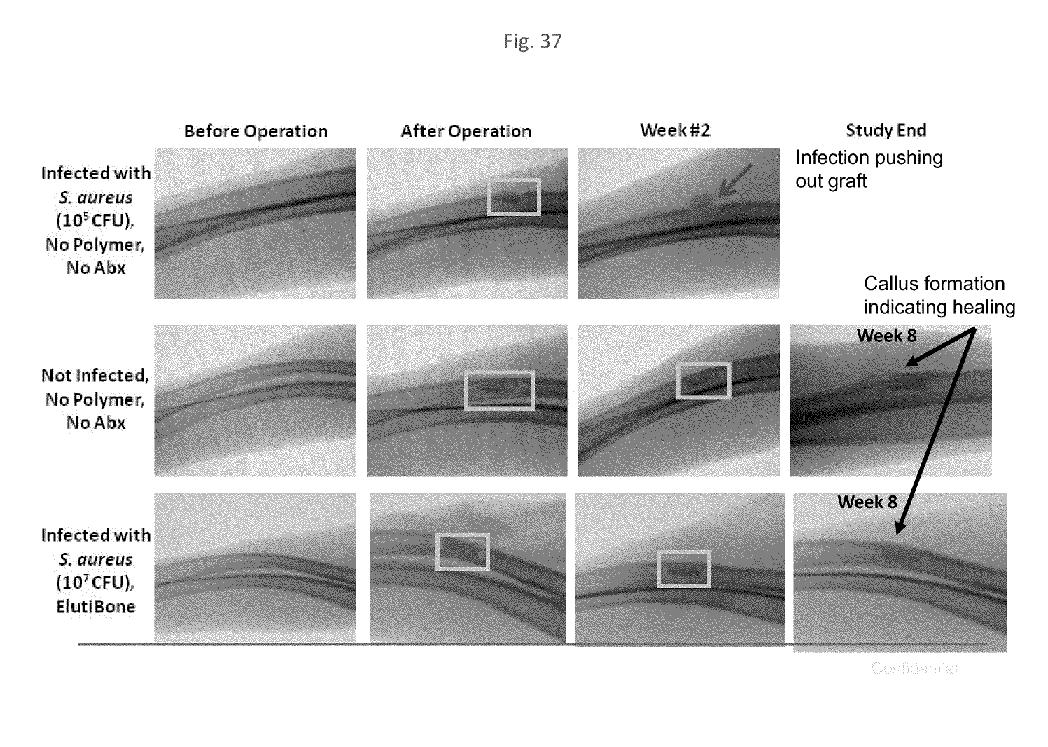

Next, photographs were taken of representative rabbits. As shown in FIG. 37, rabbits infected with S. aureus and implanted with a bone implant that was soaked in tobramycin and then coated with a polymer coating (i.e., Cohort 7) showed osseointegration of the graft at 8 weeks. (FIG. 37, bottom row labeled “infected with S. aureus (10⁷ CFU), ElutiBone). However, S. aureus infected animals implanted with a bone implant that was not soaked in tobramycin and was not coated with a polymer coating had an infected implant within two weeks (FIG. 37, top row).

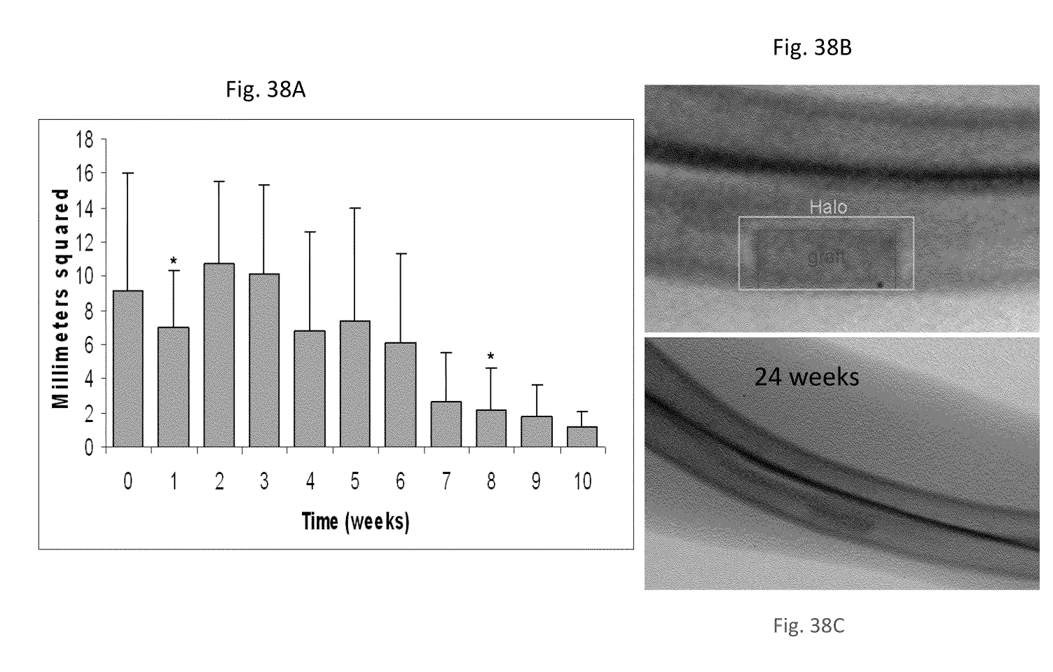

Radiographic analysis was also performed. FIG. 38A shows a bar graph showing the size of the graft of the cohort 6 animals at the indicted week post-implant. FIGS. 38B and 38C are radiographic images showing the graft in situ. A reduction in the graft area (see FIG. 38A) is consistent with the integration and loss of infection at the implantation site.

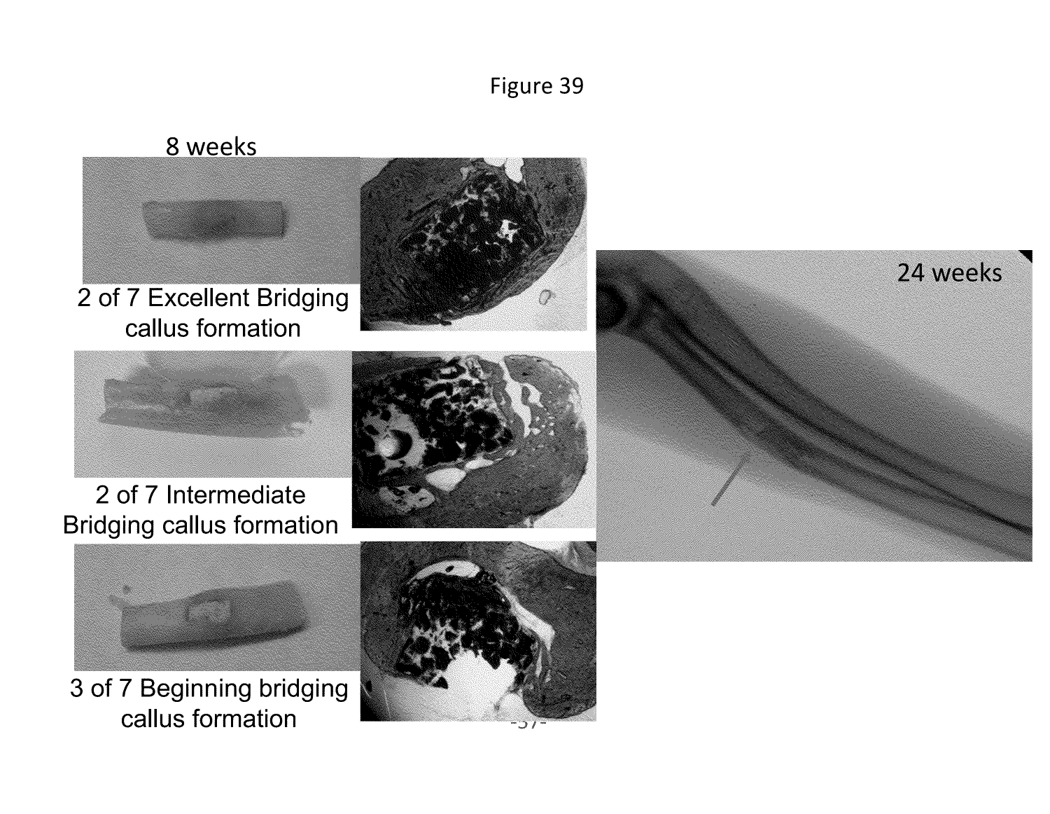

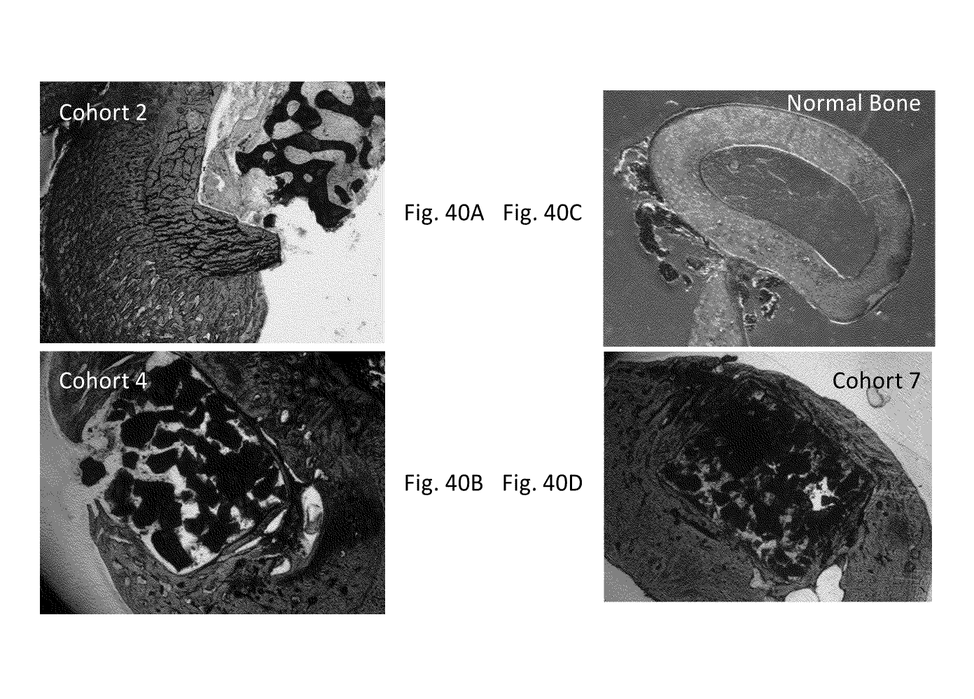

Histology analysis of the animals from cohort 6 shows that the PCL-PEG polymer coated, drug-soaked implant results in the formation of bridging calleous (“callus formation”, a typical bone healing response) as indicated by the dark bone implant being completed enveloped by red host tissue at 8 weeks (left in FIG. 39) and at 24 weeks (FIG. 39, right). FIGS. 40A-40D show the callus formation from cohort 2 animals (FIG. 40A), cohort 4 animals (FIG. 40B), and cohort 7 animals (FIG. 40D), as compared to normal bone (FIG. 40C).

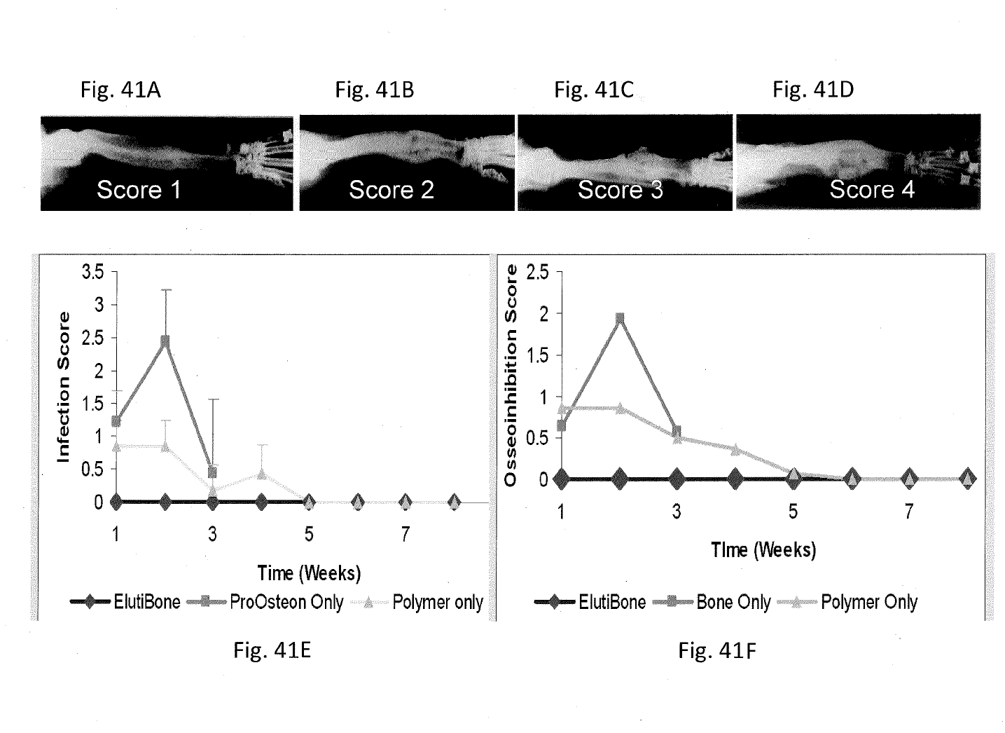

FIGS. 41A and 41B shows the radiographic analysis of infection (FIG. 41A) and osseoinhibition score (FIG. 41B) from cohort 2 (red squares), cohort 4 (yellow triangles), and cohort 6 (blue diamonds). The infection and osseoinhibition scores are shown in the radiographic images above FIGS. 41A and 41B. Note that the cohort 2 scores end at 4 weeks because the animals had to be euthanized due to massive localized infection.

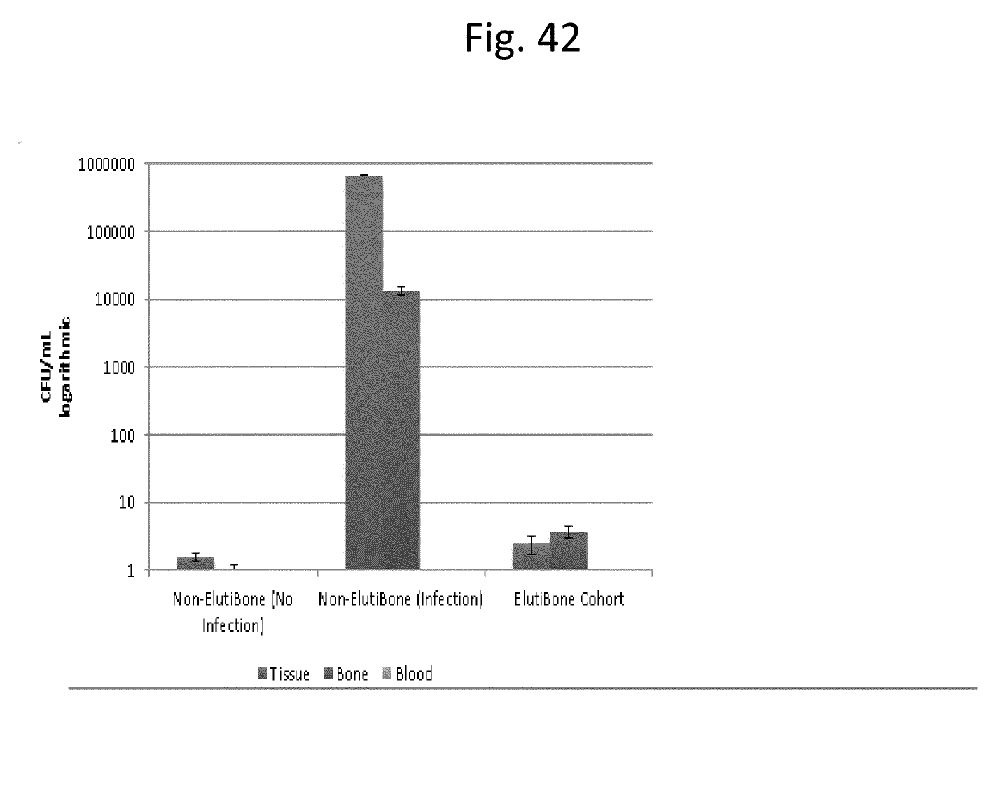

FIG. 42 is a graph showing the bacteria counts (in log scale of CFU/ml) of cohort 1 (labeled non-Elutibone; no infection); cohort 2 (labeled non-ElutiBone; infection) and cohort 7 (labeled ElutiBone cohort) in tissue (blue bars), bone (red bars), and blood (green bars).

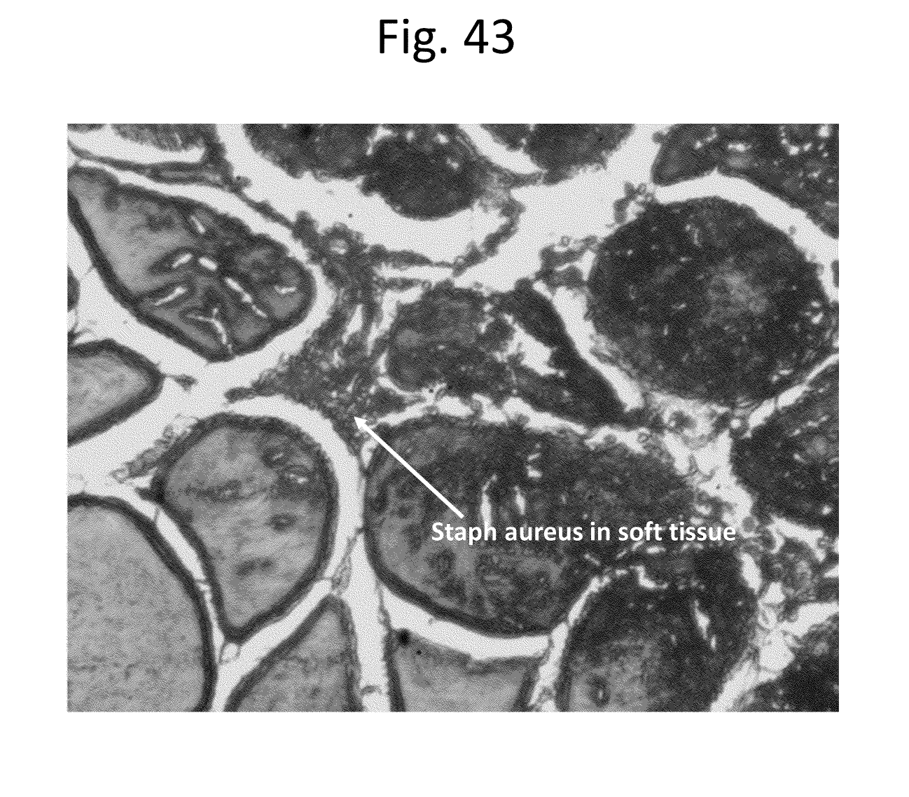

Upon euthanasia and after dissecting the right forelimb from the rabbit, the bone was fixed in 10% buffered formalin and embedded in polymethylmethacrylate (PMMA). A 10 micron section of bone was then processed for histological gram stain. FIG. 43 is a photograph of an in vivo gram stain from a cohort 4 animal. The arrow points to S. aureus infection in the soft tissue.

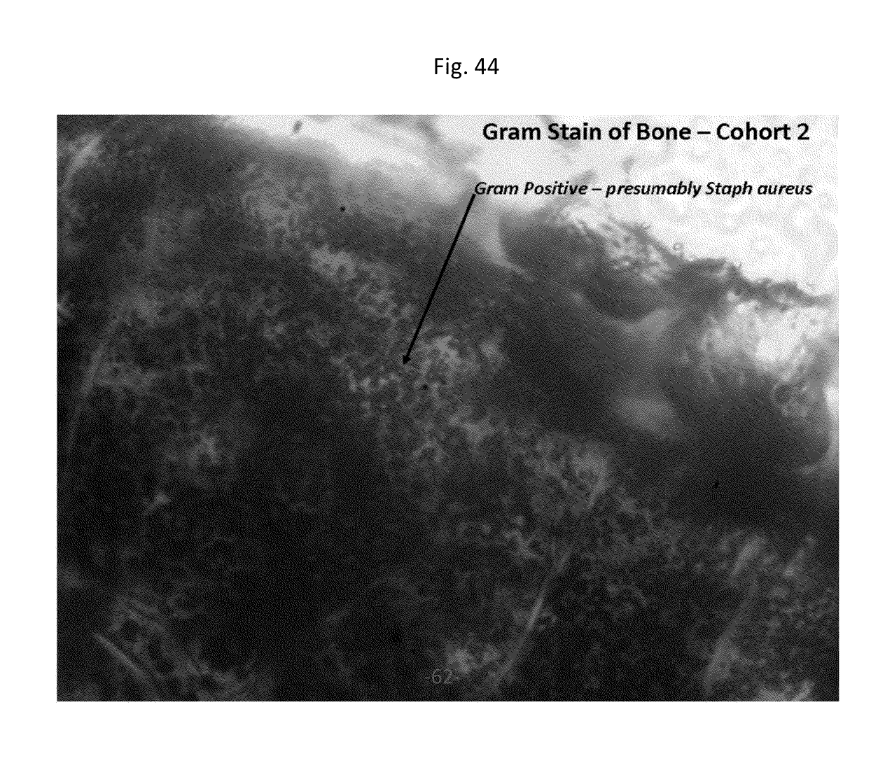

FIG. 44 is a photograph of a gram stained bone slice taken from a cohort 2 animal. The arrow points to a gram positive staining bacteria (presumably S. aureus) in the bone.

Table 6 provides a summary of the findings from this study.

| | | | | | Gram Stain |
|---|---|---|---|---|---|
| | | | | | Bone |
| 1 | No polymer, No drug | None | | NA | No |
| 2 | No polymer, No drug | 10⁵ | (CFU) | yes | yes |
| 3 | PCL-PEG, No drug | None | | NA | NA |
| 4 | PCL-PEG, No drug | 10⁵ | CFU | Yes | Yes |
| 5 | No polymer, 10% | 10⁵ | CFU | NA | A few |
| | drug soak | | | | |
| 6 | PCL-PEG, 10% drug | None | | NA | NA |
| 7 | PCL-PEG, 10% drug | 10⁵ | CFU | No | Background |

As Table 6 shows, bacteria were found to be present in both bone and soft tissue in cohort 4.

Interestingly, histological analysis of the implants from this study at the end of the study did not show the complete re-sorption of the bone by host (i.e., host bone did not completely take over the implant). This lack of osteoconduction (i.e., bone in-growth) is believed to be due to a lack within the implant of pores that are contiguously connected throughout the implant to allow penetration into the implant of host cells (e.g., osteoblasts and osteoclasts). More specifically, bone is porous, but finely ground bone necessarily loses some of the porosity present in bone before it is ground. As discussed above (see, e.g., FIGS. 9A-9C), the size and/or quantity of pores within the implant can be modulated by altering the amounts of components in the implant. For example, by suitable selection of components in the implant (and their proportions), such as drug, taking into account its molecular structure and properties, the form in which the drug is present in the implant (as either free drug or microencapsulated), type of polymer (e.g., PCL, or PCL/PEG combination), type of bone (e.g., synthetic or natural), ratio of bone to polymer, any coating of the implant, and other parameters, a desirable set of pore characteristics can be achieved. More specifically, it is believed desirable to adjust these parameters to achieve contiguous porosity in the resulting implant. Accordingly, in an embodiment of the present invention the components of the implant and their ratios in the implant are selected so as to achieve contiguous porosity in the resulting implant.

To overcome the lack of contiguous porosity (i.e., lack of interconnected pores) within the implant, in another embodiment, an additional formulation comprising a polymer component of a PCL:PEG: poly(lactide-co-glycolide) combination; a bone component of ground bone (e.g., natural or synthetic bone such as ProOsteon); and a drug component (e.g., tobramycin) is employed. In yet another embodiment, a formulation comprising a polymer component of a PCL:PEG: poly(lactide-co-glycolide) combination including a poragen such as calcium chloride; a bone component of ground synthetic bone such as ProOsteon; and a drug component of tobramycin is employed. Of course the ground synthetic bone can be replaced with ground natural bone (or synthetic bone from other sources) and the drug can be a drug other than tobramycin. Yet another alternative is to apply an implant as a liquid paste (see, e.g., Example 7 below).

A tibia osteomyelitis model in sheep is next performed at a GLP facility. Since sheep bone is similar to human bone, sheep are studied. For these studies, eleven sheep will be used per study group, and 10⁵-10⁷ S. aureus will be used to infect the sheep at the implantation site.

The results in the sheep will show that implantation of a generation 3 fabrication bone implant that fabricated with tobramycin (i.e., the tobramycin was loaded uniformly throughout the implant during the generation 3 fabrication method as per FIG. 7C), and then coated with an polymer coating that may or may not contain an antibiotic in the coating together with infection with S. aureus will be successful in stopping and preventing infection of the implant for over twelve weeks. In contrast, implantation of a bone implant that is not soaked in tobramycin and/or is not coated with an antibiotic-containing polymer coating together with infection with S. aureus will not be successful in stopping and/or preventing infection of the implant for over twelve weeks.

Taken together, these results show that the bone implant that was loaded tobramycin (i.e., the tobramycin is loaded uniformly throughout the implant) and then coated with an antibiotic-containing polymer coating is superior in stopping and preventing infection for a prolonged period of time. Table 7 provides a summary of the results described herein.

TABLE 7

| | Morselized | | | | |
|---|---|---|---|---|---|
| | bone mixed | Antibiotic | | Antibiotic | Oral or |
| | with tobramycin | loaded | Antibiotic- | Loaded | intravenous |
| | and a molten | bone | soaked | Fibrin | (IV) |
| Attribute | polymer | cement | bone chips | Glue | antibiotics |
| Antibiotic release | Yes | No | No | No | Yes |
| for over 6-8 weeks | | | | | |
| Controlled | Yes | No | No | No | No |
| antibiotic release | | | | | |
| Local delivery of | Yes | Yes | Yes | Yes | No |
| antibiotic | | | | | |
| Osteoconductive | Yes | No | Yes | No | No |

### Example 7

In this Example, an injectable bone paste using low molecular weight PCL and PEG, with the bone ceramic composite mixture at more than 50 weight % bone solid particles, and drug mixture is described.

This injectable bone paste will be fabricated as follows. 60% morselized ProOsteon will be mixed with 30% polymer (PCL (<3 kD) and PEG (<lkD) in ratios of 75-99% and 1-25% respectively) at 75° C. to create a molten paste. Up to 10% powdered tobramycin will be added to the molten mixture at which time it will be packed into a 1-5 ml syringe for sterilization. This paste could then be injected directly at the site of injury through a large gauge needle (e.g., 18-22 gauge). This formulation is anticipated to slightly harden in situ, but remain fairly viscous allowing it to remain at the site of injury and release its drug as the binding polymer matrix is degraded over time. With this low molecular weight polymer, this injectable paste may not have the same length of antibiotic release as the solid fabrications described herein (e.g., generated using the generation 2 or generation 3 fabrication method). Rather, the injectable paste fabrication may have an antibiotic release time of about 4-6 weeks.

It is expected that histological analysis of this implant after six weeks will reveal resorption of the implant by host (i.e., implant replaced by host bone).

### Example 8

In this example, the implant described herein is used in conjunction with a prosthesis.

Annual incidence of infections to orthopedic implants in the United States is substantial: 12,000 total joint infections and over 100,000 infected bone fixation implants annually. This produces a substantial cost both in terms of patient morbidity and financial coverage of these infections. All medical device-related infections are estimated to cost $1.7-4.6 billion in excess medical costs to U.S. hospitals annually. If 20% of these device-related infections could be prevented, $300-900 million in medical costs and much patient morbidity, pain and suffering could be saved.

Cement-Less Biological Bone Fixation and Implant Porosity.

Cement-less fixation represents an alternative method to popular acrylic bone cements to place and stabilize metal implants in bone. The method intends to stabilize metal implants using the patient's own direct bone-implant on-growth, on-bonding between bone and implant surface, and mechanical fixation from this interaction. The method, used in various forms since the 1980's, is intended to surpass cemented implant fixation as the method of the future—PMMA and standard thermoset cement technology will be eventually passed over in favor of cementless implant-bone bonding relying on direct bone-implant bonding. Typically, cementless fixation has been produced by host bone in-growth into carefully designed and fabricated implant pores of sufficiently large size. Porosity is critical to promote and produce this bone on-bonding fixation process with an implant. When new bone from the patient calcifies within these pores, this allows mechanical interlocking and stabilization of the bone-implant interface, eliminating the need for acrylic cements. Importantly, the ideal pore size should mimic that of native cancellous bone that ranges from 400-500 microns (dense cortical bone by comparison is only 8% porous). By contrast, most porous metallic implants (e.g., commercially pure (CP) titanium, cobalt-chrome alloys, Ti-6Al-4V alloy) have pores ranging from 100-400 microns, with 30-50% total porosity. Proper implant pores sizes and pore densities prompt enhanced bone-based fixation, achieved earlier than using fixation with allograft cortical bone, in some cases a matter of weeks.

Zimmer (Warsaw, Ind., USA) introduced their Trabecular Metal™ implants fabricated of elemental tantalum metal (a rare and highly corrosion resistant metal applied to dental implants since the 1950s) using a vapor deposition technique to create a metallic strut configuration that is similar to trabecular bone architecture. The crystalline micro- and nano-texture of a Trabecular Metal strut is conducive to direct bone apposition. Furthermore, implants fabricated from tantalum offer high porosity, allowing not only bone around implant sites to grow onto the material but also into it—a process known as osseo-incorporation or biological fixation. Zimmer's trabecular metal implants are 70-80% porous, similar to cancellous bone. Studies on dental implants containing Trabecular Metal in canine mandibular models began in 2010 and showed evidence of in-growth by maturing bone as early as two weeks after implantation. Moreover, transcortical animal implant studies have demonstrated excellent new bone in-growth of Zimmer's Trabecular Metal implants within eight weeks of surgery, promoting rapid fixation strength. According to the Zimmer company reports, human trials data are currently being collected with the first long-term results expected to be available in 2012. Zimmer has gained CE approval for another dental Trabecular Metal implant in Europe in 2011 and anticipates market approval for the USA through the Food and Drug Administration soon. Trabecular Metal has been already used fbr more than a decade in many of Zimmer's orthopedic devices.

Infection and Cementless Fixation and Porosity.

Although cementless fixation via porous metallic implants continues to provide mechanical integrity, there is an increased long-term risk of revision due to infection in hybrid and cemented implants compared to uncemented implants as evidenced by several clinical studies on total hip arthroplasties. There are considerable indications that cementing produces substantial infection risk, even acting as a nidus of infection. Hence, acute infection rates in this host post-implantation are as significant in cementless and cemented fixation in studies reported to date. Significantly, infection serves to inhibit bone generation and on-growth, limiting implant stabilization by bone growth. Limited (and clinically preliminary) evidence for more chronic infections indicates that cementless fixation infection incidence longer-term is less than for cemented. Cemented fixation notably addresses infection risk with antibiotic-containing cements, but these suffer from low fractional release and low antimicrobial capabilities long term. The presence of the cement may act as a foreign body, enhancing rates of infection after antibiotic release is exhausted after a few days post-implantation. A significant unmet clinical need and opportunity exists currently in addressing infection risk in cementless fixation with a resorbable polymer/granular bone/drug composite coating or press-fit wafer that accommodates cementless fixation while mitigating infection risk short-term. This allows bone regeneration without infection.

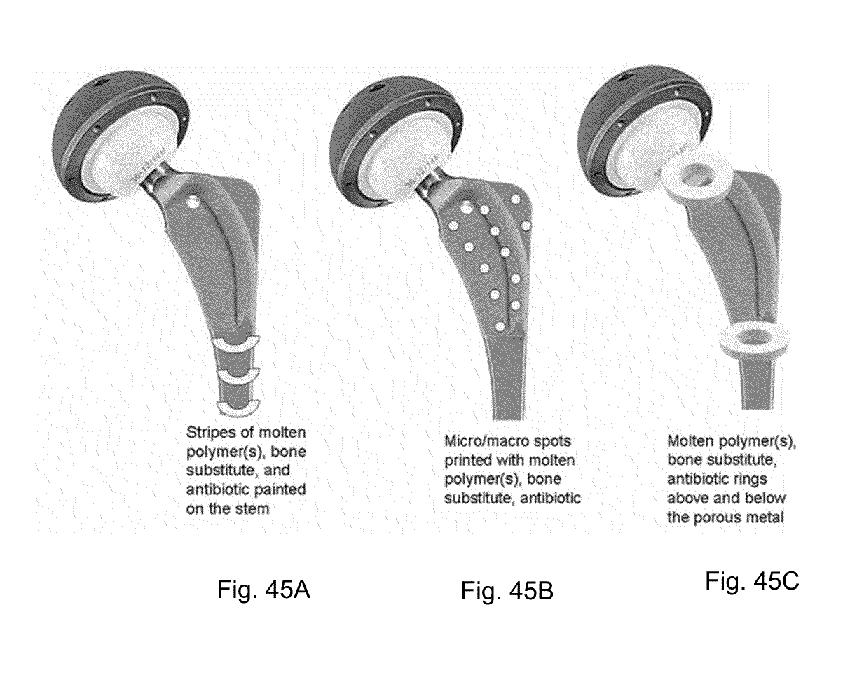

Porous metal fixation designs on implants represent a known clinical infection risk. The methods and compositions described herein as applied to cementless fixation implants may mitigate this risk for the following reasons. First, the antibiotic eluting implants described herein are composite polymer-bone graft-drug matrices. Also, the implants described herein can be patterned onto (e.g., coated onto) metallic implants to produce local high-resolution zones of antibiotic-release on or adjacent to cementless porous metal areas (see FIG. 45C). Additionally, the implant formulations described herein can be applied in microdot patterns on or around porous metal zones on implants (see FIGS. 45A and 45B). Since the implant formulations described herein can be tailored to degrade in months, this will provide antibiotic protection as bone in-growth is over the same time frame, and then fully resorb fully as bone ingrowth of host bone onto the device matures at several months post-implantation. Given the ease of manipulation of the implants described herein, the implants can be printed robotically or painted by hand or press-fit into pre-machined grooves or designated drug-release zones on metal implants and release drug for weeks while resorbing as bone in-growth occurs. Likewise, the implants can be molded or carved to meet specific dimensions or sizes (e.g., to shape the implant to fit within a specific defect site or to be placed adjacent to the prosthesis cementless fixation area). Moreover, the implants described herein, which are designed to resorb at rates commensurate with bone in-growth into porous metal, can be loaded with diverse drugs (e.g., antibiotics, growth factors, anti-inflammatory, anti-osteoporotic drugs), even in different areas of the implant using precision spray coating, printing, or press-fitting of pre-fabricated pieces.

Thus, the methods and compositions described herein provide an on-board controlled drug delivery antimicrobial solution to infection in cementless fixation and microporous metals used in orthopedic and dental implant applications. The methods and compositions described herein provide a versatile device formula which contains resorbable, clinically familiar polymers, synthetic or allograft granular bone graft materials and clinically approved drugs. This formula can be applied by spray, high-resolution patterned inkjet, molding, pre-fabrication or dip coating methods locally in resolved spatial locations on device surfaces. The implant formulations provided herein can also be shape-molded specifically for press fitting into defects or pre-designed groove sites on metallic implants.

In some embodiments, the non-limiting implants of the invention enable desired drug-graft material interaction including: 1. Molding of the bone graft composite material to specific dimensions and sizes, with a known, reliable drug load, 2. A capability to carve and shape the graft to fit specific defect sites, and 3. extended control over drug release for long time periods and subsequent antimicrobial protection throughout the duration of bone remodeling as evidenced by preclinical studies in a rabbit radial critical size defect infection model. Ultimately while releasing bactericidal concentrations of tobramycin, this antibiotic-loaded bone graft provides recognized beneficial osteoconductive potential, seeking to decrease orthopedic surgical infection incidence with improved filling of dead space and more reliable new bone formation.

Additional references include the following, all of which are incorporated herein by reference in their entireties.
Kalorama Information Market Intelligence Report, Orthopedic Biomaterials: World Market, September 2007.
Millenium Research Group, US Markets for Orthopedic Biomaterials, RPUS200B08, April 2008.
Merrill, C. & Elixhauser, A., Hospital Stays Involving Muskuloskeletal Procedures, 1997-2005, HCUP Statistical Brief #34.
Kurtz, S., Projections of Primary and Revision Hip and Knee Arthroplasty in the US from 2005 to 2030, J Bone Joint Surg Am 2007; 89:780-5.
Medtech Insight, U.S. Surgical Procedure Volumes, March 2009.
Jiranek, W., Hanssen, A. & Greenwald, A., Antibiotic Loaded Bone Cement for Infection Prophylaxis in Total Joint Replacement, J Bone Joint Surg Am 2006; 88:2487-2500.
Kurtz, S. et al, Infection Burden for Hip and Knee Arthroplasty in the U.S., J Arthroplasty, Vol. 23 No. 7, 2008.
Bozic, K., et al, Epidemiology of Revision Total Knee Arthroplasty in the U.S., Clin Orthop Relat Res (2010) 468:45-47.
Harris, I., & Lyons, M., Reoperation Rate in Diaphyseal Tibia Fractures, ANZ J. Surg. 2005: 1041-1044.
Gallazzi et al., Early Radiological Diagnosis and Differential Diagnosis of Infection in Orthopaedic Surgery in Infection and Local Treatment in Orthopedic Surgery, Springer 2007.

The embodiments of the invention described above are intended to be merely exemplary; numerous variations and modifications will be apparent to those skilled in the art. All such variations and modifications are intended to be within the scope of the present invention as defined in any appended claims.

What is claimed is:

1. An implant comprising a uniform mixture including degradable polymer, bone, and a drug.

2. The implant of claim 1, wherein the drug comprises an antibiotic.

3. The implant of claim 1, configured so that upon implantation of the implant at an implantation site, the drug diffuses from the implant at a therapeutic level at the implantation site for at least eight weeks.

4. The implant of claim 1, configured so that upon implantation of the implant at an implantation site, the drug diffuses from the implant at a therapeutic level at the implantation site for at least ten weeks.

5. The implant of claim 1, configured so that upon implantation of the implant at an implantation site, the drug diffuses from the implant at a therapeutic level at the implantation site for at least twelve weeks.

6. The implant of claim 1, wherein the implant is a solid.

7. The implant of claim 1, wherein the implant is molded.

8. The implant of claim 6, wherein the implant is carveable, so that it may be shaped prior to implantation.

9. The implant of claim 1, wherein the implant is shaped for use with an implantable prosthesis.

10. The implant of claim 1, wherein the implant is a liquid.

11. The implant of claim 1, wherein the implant is a paste.

12. The implant of claim 1, wherein the implant is a putty.

13. The implant of claim 1, wherein the bone is natural bone.

14. The implant of claim 1, wherein the bone is synthetic bone.

15. The implant of claim 1, wherein the implant is contiguously porous.

16. The implant of claim 1, wherein the implant is a coating on an implantable prosthesis.

17. The implant of claim 1, configured so that upon implantation of the implant, the drug diffuses from the implant in a manner to provide a first bolus after a first period of time following implantation and a second bolus after a second period of time following implantation.

18. The implant of claim 17, wherein the first period is about one week and the second period is about five weeks.

19. The implant of claim 17, wherein the first period is about one day and the second period is between about three weeks and about six weeks.

20. The implant of claim 1, wherein the degradable polymer comprises a polycaprolactone (PCL) polymer.

21. The implant of claim 1, wherein the degradable polymer comprises a polyethylene glycol (PEG) polymer.

22. The implant of claim 1, wherein the degradable polymer comprises a poly(lactide-co-glycolide) polymer.

23. The implant of claim 1, wherein the implant further comprises a poragen.

24. The implant of claim 1, wherein the bone is present in the uniform mixture in a first quantity by weight and the degradable polymer is present in the uniform mixture in a second quantity by weight, wherein the first quantity is greater than the second quantity.

25. The implant of claim 24, wherein the first quantity is at least 1.125 times larger than the second quantity.

26. The implant of claim 24, wherein the first quantity is at least 1.25 times larger than the second quantity.

27. The implant of claim 24, wherein the first quantity is at least 1.5 times larger than the second quantity.

28. The implant of claim 24, wherein the first quantity is at least two times larger than the second quantity.

29. The implant of claim 24, wherein the first quantity is at least 2.25 times larger than the second quantity.

30. The implant of claim 24, wherein the first quantity is at least 2.5 times larger than the second quantity.

31. A method of making a solid implant, the method comprising:
making a uniform mixture including degradable polymer, bone, and a drug;
forming the mixture into a desired shape; and
curing the shaped mixture to form a solid implant.

32. The method of claim 31, wherein curing the shaped mixture includes subjecting it to heat.

33. The method of claim 31, wherein the bone is present in the uniform mixture in a first quantity by weight and the degradable polymer is present in the uniform mixture in a second quantity by weight, wherein the first quantity is greater than the second quantity.

34. The method of claim 33, wherein the first quantity is at least 1.125 times larger than the second quantity.

35. The method of claim 33, wherein the first quantity is at least 1.25 times larger than the second quantity.

36. The method of claim 33, wherein the first quantity is at least 1.5 times larger than the second quantity.

37. The method of claim 33, wherein the first quantity is at least two times larger than the second quantity.

38. The method of claim 33, wherein the first quantity is at least 2.25 times larger than the second quantity.

39. The method of claim 33, wherein the first quantity is at least 2.5 times larger than the second quantity.

40. The method of claim 31, wherein the implant is contiguously porous.

41. An implantable bone void filler comprising a polymer component comprising a polycaprolactone (PCL) polymer, an antibiotic, and a bone component.

42. The filler of claim 41, wherein the antibiotic is selected from the group consisting of tobramycin, ciprofloxacin, and vancomycin.

43. The filler of claim 41, wherein the polymer component further comprises a polyethylene glycol (PEG) polymer.

44. The filler of claim 41, wherein the polymer component further comprises a poly(lactide-co-glycolide) polymer.

45. The filler of claim 41, wherein the filler further comprises a poragen.

46. The filler of claim 41, wherein the bone component comprises a bone fragment.

47. The filler of claim 41, wherein the bone component comprises a ground bone.
